(19) United States
(12) Patent Application Publication
(10) Pub. No.: US 2003/0139358 A1
(43) Pub. Date: Jul. 24, 2003

(54) Novel human proteins, polynucleotides encoding them and methods of using the same

(76) Inventors: Kimberly A. Spytek, New Haven, CT (US); Muralidhara Padigaru, Branford, CT (US); Kumud Majumder, Stamford, CT (US); John R. MacDougall, Hamden, CT (US); David J. Stone, Guilford, CT (US); Esha A. Gangolli, Madison, CT (US); Steven K. Spaderna, Berlin, CT (US); Glennda Smithson, Branford, CT (US)

Correspondence Address: MINTZ, LEVIN, COHN, FERRIS,, GLOVSKY and POPEO, P.C., One Financial Center, Boston, MA, 02111, US

(21) Appl. No.: 09/849,138

(22) Filed: May 4, 2001

Related U.S. Application Data
(60) Provisional application No. 60/201,951, filed on May 5, 2000.
(60) Provisional application No. 60/215,857, filed on Jul. 3, 2000.
(60) Provisional application No. 60/265,162, filed on Jan. 30, 2001.
(60) Provisional application No. 60/203,109, filed on May 8, 2000.
(60) Provisional application No. 60/203,295, filed on May 11, 2000.
(60) Provisional application No. 60/210,055, filed on Jun. 7, 2000.
(60) Provisional application No. 60/204,064, filed on May 12, 2000.
(60) Provisional application No. 60/204,063, filed on May 12, 2000.
(60) Provisional application No. 60/204,062, filed on May 12, 2000.
(60) Provisional application No. 60/203,838, filed on May 12, 2000.
(60) Provisional application No. 60/203,839, filed on May 12, 2000.
(60) Provisional application No. 60/204,089, filed on May 15, 2000.
(60) Provisional application No. 60/204,276, filed on May 16, 2000.

(51) Int. Cl. A61K 48/00; A61K 38/17; C12Q 1/68; G01N 33/53; C07H 21/04; C12P 21/02; C12N 9/00; C12N 5/06

(52) U.S. Cl. 514/44; 514/12; 435/6; 435/7.1; 435/69.1; 435/183; 435/320.1; 536/23.2

(57) ABSTRACT

The invention provides polypeptides, designated herein as POLYX polypeptides, as well as polynucleotides encoding POLYX polypeptides, and antibodies that immunospecifically-bind to POLYX polypeptide or polynucleotide, or derivatives, variants, mutants, or fragments thereof. The invention additionally provides methods in which the POLYX polypeptide, polynucleotide, and antibody are used in the detection, prevention, and treatment of a broad range of pathological states.

### RELATED APPLICATIONS

This application claims priority to U.S. No. 60/201,951(21402-001), filed May 5, 2000; No. 60/215,857 (21402-001A), filed Jul. 3, 2000; No. 60/265,162 (21402-001B), filed Jan. 30, 2001; No. 60/203,109 (21402-002), filed May 8, 2000; No. 60/203,295 (21402-003), filed May 11, 2000; No. 60/210,055 (21402-003A), filed Jun. 7, 2000; No. 60/204,064 (21402-004), filed May 12, 2000; No. 60/204,063 (21402-005), filed May 12, 2000; No. 60/204,062 (21402-006), filed May 12, 2000; No. 60/203,838 (21402-007) filed May 12, 2000; No. 60/203,839 (21402-008), filed May 12, 2000; No. 60/204,089 (21402-009), filed May 15, 2000; and No. 60/204,276 (21402-010), filed May 16, 2000. The contents of these applications are incorporated by reference in their entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: myosin light chain kinase (MLCK), calgizzarin, beta thymosin, ras suppressor, cerebellin, lymphotactin, zinc transporter, tetracycline transporter and macrophage stimulating protein (MSP) precursor. More particularly, the invention relates to nucleic acids encoding novel polypeptides, as well as vectors, host cells, antibodies, and recombinant methods for producing these nucleic acids and polypeptides.

The MLCK family of proteins are responsible for catalyzing the phosporylation of the light chain of myosin during the contraction of smooth muscle. Thus, the MLCK proteins serve as a key enzyme in muscle contraction and have been shown by immunohistology to be present in neurons and glia. Phosphorylation of myosin II regulatory light chains (RLC) by Ca2+/calmodulin (CAM)-dependent myosin light chain kinase is a critical step in the initiation of smooth muscle and non-muscle cell contraction. Post-translational modifications to MLCK down-regulate enzyme activity, suppressing RLC phosphorylation, myosin II activation and tension development. The proteins of the MLCK family have been shown to be useful in potential therapeutic applications implicated in various pathologies/disorders such as, for example, muscular dystrophy, Lesch-Nyhan syndrome and Myasthenia gravis.

The calgizzarin protein is purified from proteins of the S100 family of proteins, which belong to the large group of EF-hand calcium-binding proteins. The expression of human calgizzarin has been found to be remarkably elevated in colorectal cancers compared with that in normal colorectal mucosa. Calgizzarin has also been shown to be one of several genes expressed in breast cancer-derived metastatic axillary lymph nodes but not in normal lymph nodes or breast fibroadenomas. As such, calgizzarin proteins have been shown to be useful in potential therapeutic applications implicated in cancer, neuropsychiatric disorders, medullary cystic kidney disease and anemia.

The beta thymosin family of proteins and polypeptides induce the expression of terminal deoxynucleotidyl transferase activity in vivo and in vitro, inhibit the migration of macrophages, and stimulate the secretion of hypothalmic leuteinizing hormone-releasing hormone. Thymosin-beta-4, a member of the beta thymosin family has been shown to be a potent wound healing factor. Beta thymosin proteins have also been found to be useful in potential therapeutic applications implicated in cancers, immunological and autoimmune disorders, angiogenesis, modulation of apoptosis, neurodegenerative and neuropsychiatric disorders, age-related disorders and other pathological disorders.

The Ras Suppressor Protein is capable of inhibiting v-Ras transformation. The ras suppressor protein has been shown to be useful in potential therapeutic applications implicated in various cancers including but not limited to leukemia, melanomas, carcinomas, sarcomas, bladder, mammary, renal-pelvic, ovarian, lung and colon cancer, and human solid tumors and urinary tract tumors; and other types of neoplastic disorders and/or other pathologies and disorders.

Cerebellin is a truncated derivative of precerebellin, a large protein with distant homology to the noncollagen domain of complement component C1qB. Immunoreactive cerebellin has been detected in every region of the brain studied, with the highest concentrations found in the hemisphere of the cerebellum and the vermis of the cerebellum. Immunoreactive cerebellin was also detected in the pituitary, the spinal cord and the normal parts of adrenal glands and some tumor tissues. Cerebellin proteins may have therapeutic applications in olivopontocerebellar atrophy (OPCA), Shy-Drager syndrome, ‘staggerer syndrome’ and various cancers such as, for example, brain and adrenal gland tumors, including phaeochromocytomas, cortisol-producing adrenocortical adenomas, ganglioneuroblastomas and neuroblastomas.

The lymphotactin-like family proteins are a class of lymphocyte-specific chemokine, which are useful in potential therapeutic applications implicated in development, homeostasis, and function of the immune system. Lymphotactin-like proteins also have effects on cells of the central nervous system as well as on endothelial cells involved in angiogenesis or angiostasis and/or other pathologies and disorders.

The zinc transporter proteins are implicated in the transport of zinc, an important trace metal, in organisms with zinc deficiencies. The zinc transporter proteins are thus useful in potential therapeutic applications implicated in disorders related to zinc deficiencies including immune challenge, oxidative damage, dermatitis, alopecia, stunted growth or deficiencies of varying levels of other metals that compete for these transporters.

The family of macrophage-stimulating protein (MSP) precursors are also known as hepatocyte growth factor-like proteins (HGFL), and are structurally related to hepatocyte growth factor/scatter factor (HGF/SF). HGF/SF and MSP define a novel growth factor family whose members share the domain structure and the proteolytic process of activation of the blood proteinase precursor plasminogen. MSP and its tyrosine kinase receptor RON have been implicated in metastatic breast cancer. Therefore, the MSP family of proteins are useful in diagnostic and therapeutic applications implicated in disorders relating to cancer and metastatic potential.

The tetracycline transporter protein family is conserved from bacteria to humans, and is important in multidrug resistance. Therefore, new members of the tetracycline transporter protein family are useful in diagnostic and therapeutic applications implicated in disorders relating to multidrug resistance important in bacterial infections, cancer and liver disease.

### SUMMARY OF THE INVENTION

The invention is based, in part, upon the discovery of novel nucleic acids and secreted polypeptides encoded thereby. The nucleic acids and polypeptides are collectively referred to herein as “POLYX” nucleic acids and polypeptides.

Accordingly, in one aspect, the invention includes an isolated nucleic acid that encodes a POLYX polypeptide, or a fragment, homolog, analog or derivative thereof. For example, the nucleic acid can encode a polypeptide at least 85% identical to a polypeptide comprising the amino acid sequences of SEQ ID NO: 2, 4, 6, 8, 10, 12, 14, 16, 18, 20, 22, 24, 26 and/or 28. The nucleic acid can be, e.g., a genomic DNA fragment or a cDNA molecule. In some embodiments, the invention provides an isolated nucleic acid molecule that includes the nucleic acid sequence of any of SEQ ID NO: 1, 3, 5, 7, 9, 11, 13, 15, 17, 19, 21, 23, 25 and/or 27.

Also included within the scope of the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a POLYX nucleic acid and a pharmaceutically acceptable carrier or diluent.

In a further aspect, the invention includes a substantially purified POLYX polypeptide, e.g., any of the POLYX polypeptides encoded by a POLYX nucleic acid, and fragments, homologs, analogs, and derivatives thereof. The invention also includes a pharmaceutical composition that includes a POLYX polypeptide and a pharmaceutically acceptable carrier or diluent.

In a still a further aspect, the invention provides an antibody that binds specifically to a POLYX polypeptide. The antibody can be, e.g., a monoclonal or polyclonal antibody, and fragments, homologs, analogs, and derivatives thereof. The invention also includes a pharmaceutical composition including POLYX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a POLYX polypeptide by providing a cell containing a POLYX nucleic acid, e.g., a vector that includes a POLYX nucleic acid, and culturing the cell under conditions sufficient to express the POLYX polypeptide encoded by the nucleic acid. The expressed POLYX polypeptide is then recovered from the cell. Preferably, the cell produces little or no endogenous POLYX polypeptide. The cell can be, e.g., a prokaryotic cell or eukaryotic cell.

The invention is also directed to methods of identifying a POLYX polypeptide or nucleic acids in a sample by contacting the sample with a compound that specifically binds to the polypeptide or nucleic acid, and detecting complex formation, if present.

The invention further provides methods of identifying a compound that modulates the activity of a POLYX polypeptide by contacting a POLYX polypeptide with a compound and determining whether the POLYX polypeptide activity is modified.

The invention is also directed to compounds that modulate POLYX polypeptide activity identified by contacting a POLYX polypeptide with the compound and determining whether the compound modifies activity of the POLYX polypeptide, binds to the POLYX polypeptide, or binds to a nucleic acid molecule encoding a POLYX polypeptide.

In a another aspect, the invention provides a method of determining the presence of, or predisposition to a POLYX-associated disorder in a subject. The method includes providing a sample from the subject and measuring the amount of POLYX polypeptide in the subject sample. The amount of POLYX polypeptide in the subject sample is then compared to the amount of POLYX polypeptide in a control sample. An alteration in the amount of POLYX polypeptide in the subject protein sample relative to the amount of POLYX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the POLYX is detected using a POLYX antibody.

In a further aspect, the invention provides a method of determining the presence of, or predisposition to, a POLYX-associated disorder in a subject. The method includes providing a nucleic acid sample (e.g., RNA or DNA, or both) from the subject and measuring the amount of the POLYX nucleic acid in the subject nucleic acid sample. The amount of POLYX nucleic acid sample in the subject nucleic acid is then compared to the amount of POLYX nucleic acid in a control sample. An alteration in the amount of POLYX nucleic acid in the sample relative to the amount of POLYX in the control sample indicates the subject has a tissue proliferation-associated disorder.

In a still further aspect, the invention provides a method of treating or preventing or delaying a POLYX-associated disorder. The method includes administering to a subject in which such treatment or prevention or delay is desired a POLYX nucleic acid, a POLYX polypeptide, or a POLYX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides novel polynucleotides and the polypeptides encoded thereby. The invention is based in part on the discovery of nucleic acids encoding 14 proteins that are novel members of the following protein families: myosin light chain kinase (MLCK), calgizzarin, beta thymosin, ras suppressor, cerebellin, lymphotactin, zinc transporter, tetracycline transporter and macrophage stimulating protein (MSP) precursor. These nucleic acids, and their associated polypeptides, antibodies and other compositions are referred to as POLY1, POLY2, POLY3 through POLY14, respectively. These sequences are collectively referred to as “POLYX nucleic acids” or “POLYX polynucleotides” (where X is an integer between 1 and 14) and the corresponding encoded polypeptide is referred to as a “POLYX polypeptide” or “POLYX protein”.

POLY1-4 are novel members of the MLCK family; POLY5-6 are novel members of the calgizzarin family; POLY7-8 are novel members of the beta thymosin family; POLY9 is a novel member of the ras suppressor protein family; POLY 10 is a novel member of the cerebellin family; POLY11 is a novel member of the lymphotactin family; POLY12 is a novel member of the zinc transporter protein family, POLY13 is a novel member of the macrophage stimulating protein (MSP) precursor family and POLY14 is a novel member of the tetracycline transporter family.

Table 1 provides a cross-reference between a POLYX nucleic acid or polypeptide of the invention, a table disclosing a nucleic acid and encoded polypeptide that is encompassed by an indicated POLYX nucleic acid or polypeptide of the invention, and a corresponding sequence identification number (SEQ ID NO:). Also provided is a CuraGen internal Clone Identification Number for the disclosed nucleic acid and encoded polypeptides. Unless indicated otherwise, reference to a “Clone” herein refers to a discrete in silico nucleic acid sequence.

TABLE 1

| | | | | |
|---|---|---|---|---|
| | | | SEQ ID | |
| | | | NO: | |
| | POLYX | Table | Nucleic | SEQ ID NO: |
| Clone | Number | Number | Acid | Polypeptide |

| | | | | |
|---|---|---|---|---|
| 20483634_EXT1 | 1 | 2 | 1 | 2 |
| SC87372923-1_EXT | 2 | 3 | 3 | 4 |
| CG51448-04 | 3 | 4 | 5 | 6 |
| CG51448-03 OR | 4 | 5 | 7 | 8 |
| CG51448-02 OR | | | | |
| 153574419 _tpn_REV | | | | |
| COMP | | | | |
| AC026105_A | 5 | 6 | 9 | 10 |
| GMdj130L23_A | 6 | 7 | 11 | 12 |
| AP001591_A | 7 | 8 | 13 | 14 |
| AC025535_B | 8 | 9 | 15 | 16 |
| GM87333647_A | 9 | 10 | 17 | 18 |
| ba458e15_A | 10 | 11 | 19 | 20 |
| GM87593625_A | 11 | 12 | 21 | 22 |
| GM87756960_A | 12 | 13 | 23 | 24 |
| GM105274478_A | 13 | 14 | 25 | 26 |
| 3102960_EXT | 14 | 15 | 27 | 28 |

POLYX nucleic acids, POLYX polypeptides, POLYX antibodies, and related compounds, are useful in a variety of applications and contexts. For example, various POLYX nucleic acids and polypeptides according to the invention are useful, inter alia, as novel members of the protein families according to the presence of domains and sequence relatedness to previously described proteins.

POLYX nucleic acids and polypeptides according to the invention can also be used to identify cell types based on the presence or absence of various POLYX nucleic acids according to the invention. Additional utilities for POLYX nucleic acids and polypeptides are discussed below.

POLY1-POLY4

Myosin Light Chain Kinase (MLCK) Nucleic Acids and Proteins

POLY1-4 nucleic acids and proteins are members of the myosin light chain (MLCK) family. The MLCK family of proteins are responsible for catalyzing the phosporylation of the light chain of myosin during the contraction of smooth muscle. Thus, the MLCK proteins serve as a key enzyme in muscle contraction and have been shown by immunohistology to be present in neurons and glia. The cDNA for human myosin light chain kinase has been cloned from hippocampus and shown to encode a protein sequence 95% similar to smooth muscle MLCKs but less than 60% similar to skeletal muscle MLCKs. The cDNA clone detected two RNA transcripts in human frontal and entorhinal cortex, in hippocampus, and in jejunum, one corresponding to MLCK and the other probably to telokin, the carboxy-terminal 154 residues of MLCK expressed as an independent protein in smooth muscle. The levels of expression has been shown to be lower in brain than in smooth muscle. The acidic C-terminus of all MLCKs from both brain and smooth muscle resembles the C-terminus of tubulins. By PCR and Southern blotting using 2 somatic cell hybrid panels, the MLCK gene has been localized to 3cen-q21. Since the MLCK disclosed herein is an MLCK, the chromosomal locus has been assigned as Chromosome 3cen-q21.

Phosphorylation of myosin II regulatory light chains (RLC) by Ca2+/calmodulin (CAM)-dependent myosin light chain kinase is a critical step in the initiation of smooth muscle and non-muscle cell contraction. Post-translational modifications to MLCK down-regulate enzyme activity, suppressing RLC phosphorylation, myosin II activation and tension development.

Novel members of the MLCK family, POLY1-POLY4, are described in detail below. These nucleic acids and proteins function as described above, and therefore are useful in potential therapeutic applications implicated in various pathologies/disorders such as, for example, musclular dystrophy, Lesch-Nyhan syndrome and Myasthenia gravis.

The protein similarity information, expression pattern, cellular localization, and map location for POLY1-POLY4 discussed below suggest that these MLCK-like proteins have important structural and/or physiological functions characteristic of the MLCK family. Therefore, the nucleic acids and proteins of the invention are useful in potential diagnostic and therapeutic applications, e.g. diagnosis and therapy of neurological diseases and/or disorders, and as research tools. Additionally, POLY 1-POLY4 have applications in the diagnosis and/or treatment of various diseases and disorders. For example, the compositions of POLY1-POLY4 will have efficacy for the treatment of patients suffering from: musclular dystrophy, pseudohypertophic progressive, Duchenne and Becker types; musclular disorders, Lesch-Nyhan syndrome and Myasthenia gravis.

These materials are further useful in the generation of antibodies that bind immunospecifically to the novel substances of the invention for use in diagnostic and/or therapeutic methods.

POLY1

A novel nucleic acid was identified that is comprised of 1788 nucleotides (SEQ ID NO: 1), and which encodes a novel myosin light chain kinase-like protein and is shown in Table 2A. An open reading frame was identified beginning with an ATG initiation codon at nucleotides 1-3 and ending with a TGA codon at nucleotides 1786-1788. The start and stop codons are in bold letters. The encoded protein having 608 amino acid residues (SEQ ID NO:2) is presented using the one-letter code in Table 2B.

TABLE 2A
The nucleotide sequence of POLY1.

| | |
|---|---|
| >20483634 | |
| ATGGCGACAGAAAATGGAGCAGTTGAGCTGGGAATTCAGAACCCATCAACAGACAAGGCACCTAA | (SEQ ID NO:1) |
| AGGTCCCACAGGTGAAAGACCCCTGGCTGCAGGGAAAGACCCTGGCCCCCCAGACCCAAAGAAAG | |
| CTCCGGATCCACCCACCCTGAAGAAAGATGCCAAAGCCCCTGCCTCAGAGAAAGGGGATGGTACCC | |
| TGGCCCAACCCTCAACTAGCAGCCAAGGCCCCAAAGGAGAGGGTGACAGGGGCGGGGGGCCCGCG | |
| GAGGGCAGTGCTGGGCCCCCGGCAGCCCTGCCCCAGCAGACTGCGACACCTGAGACGAGCGTCAAG | |
| AAGCCCAAGGCTGAGCAGGGAGCCTCAGGCAGCCAGGATCCTGGAAAGCCCAGGGTGGGCAAGAA | |
| GGCAGCAGAGGGCCAAGCAGCAGCCAGGAGGGGCTCACCTGCCTTTCTGCATAGCCCCAGCTGTCC | |
| TGCCATCATCTCCAGTTCTGAGAAGCTGCTGGCCAAGAAGCCCCCAAGCGAGGCATCAGAGCTCAC | |
| CTTTGAAGGGGTGCCCATGACCCACAGCCCCACGGATCCCAGGCCAGCCAAGGCAGAAGAAGGAA | |
| AGAACATCCTGGCAGAGAGCCAGAAGGAAGTGGGAGAGAAAACCCCAGGCCAGGCTGGCCAGGCT | |
| AAGATGCAAGGGGACACCTCGAGGGGGATTGAGTTCCAGGCTGTTCCCTCAGAGAAATCCGAGGTG | |
| GGGCAGGCCCTCTGTCTCACAGCCAGGGAGGAGGACTGCTTCCAGATTTTGGATGATTGCCCGCCA | |
| CCTCCGGCCCCCTTCCCTCACCGCATGGTGGAGCTGAGGACCGGGAATGTCAGCAGTGAATTCAGT | |
| ATGAACTCCAAGGAGGCGCTCGGAGGGGGCAAGTTTGGGGCAGTCTGTACCTGCATGGAGAAAGC | |
| CACAGGCCTCAAGCTGGCAGCCAAGGTCATCAAGAAACAGACTCCCAAAGACAAGGAAATGGTGT | |
| TGCTGGAGATTGAGGTCATGAACCAGCTGAACCACCGCAATCTGATCCAGCTGTATGCAGCCATCG | |
| AGACTCCGCATGAGATCGTCCTGTTCATGGAGATCGAGGGCGGAGAGCTCTTCGAGAGGATTGTGG | |
| ATGAGGACTACCATCTGACCGAGGTGGACACCATGGTGTTTGTCAGGCAGATCTGTGACGGGATCC | |
| TCTTCATGCACAAGATGAGGGTTTTGCACCTGGACCTCAAGCCAGAGAACATCCTGTGTGTCAACAC | |
| CACCGGGCATTTGGTGAAGATCATTGACTTTGGCCTGGCACGGAGGTACCACAACCCCAACGAGAA | |
| GCTGAAGGTGAACTTTGGGACCCCAGAGTTCCTGTCACCTGAGGTGGTGAATTATGACCAAATCTCC | |
| GATAAGACAGACATGTGGAGTATGGGGGTGATCACCTACATGCTGCTGAGCGGCCTCTCCCCCTTC | |
| CTGGGAGATGATGACACAGAGACCCTAAACAACGTTCTATCTGGCAACTGGTACTTTGATGAAGAG | |
| ACCTTTGAGGCCGTATCAGACGAGGCCAAAGACTTTGTCTCCAACCTCATCGTCAAGGACCAGGCC | |
| CGGATGAACGCTGCCCAGTGTCTCGCCCATCCCTGGCTCAACAACCTGGCGGAGAAAGCCAAACGC | |
| TGTAACCGACGCCTTAAGTCCCAGATCTTGCTTAAGAAATACCTCATGAAGAGGCGCTGGAAGAAA | |
| AACTTCATTGCTGTCAGCGCTGCCAACCGCTTCAAGAAGATCAGCAGCTCGGGGGCACTGATGGCT | |
| CTGGGGGTCTGA | |

TABLE 2B
Protein sequence encoded by the coding sequence shown in TABLE 2A

| | |
|---|---|
| Frame: +1-Nucleotide 1 to 1785-595 amino acid reading frame- | |
| MATENGAVELGLQNPSTDKAPKGPTGERPLAAGKDPGPPDPKKAPDPPTLKKDAKAPASEKGDGTLAQP | (SEQ ID NO:2) |
| STSSQGPKGEGDRGGGPAEGSAGPPAALPQQTATPETSVKKPKAEQGASGSQDPGKPRVGKKAAEGQA | |
| AARRGSPAFLHSPSCPAIISSSEKLLAKKPPSEASELTFEGVPMTHSPTDPRPAKAEEGKNILAESQKEVGE | |
| KTPGQAGQAKMQGDTSRGIEFQAVPSEKSEVGQALCLTAREEDCFQILDDCPPPPAPFPHRMVELRTGN | |
| VSSEFSMNSKEALGGGKFGAVCTCMEKATGLKLAAKVIKKQTPKDKEMVLLEIEVMNQLNHRNLIQLY | |
| AAIETPHEIVLFMEIEGGELFERIVDEDYHLTEVDTMVFVRQICDGILFMHKMRVLHLDLKPENILCVNTT | |
| GHLVKIIDFGLARRYHNPNEKLKVNFGTPEFLSPEVVNYDQISDKTDMWSMGVITYMLLSGLSPFLGDD | |
| DTETLNNVLSGNWYFDEETFEAVSDEAKDFVSNLIVKDQARMNAAQCLAHPWLNNLAKAKRCNRRL | |
| KSQILLKKYLMKRRWKKNFIAVSAANRFKKISSSGALMALGV | |

In a search of sequence databases, it was found, for example, that the nucleic acid sequence (SEQ ID NO: 1) of this invention has 1275 of 1577 bases (80%) identical to a Oryctolagus cuniculus MYOSINE LIGHT CHAIN KINASE mRNA (GENBANK-ID: RABMLCKA|acc: J05194). The full amino acid sequence of the protein of the invention was found to have 525 of 608 amino acid residues (86%) identical to, and 546 of 608 residues (89%) similar to, the 608 amino acid residue MLCK protein from O.cuniculus (ptnr:PIR-ID: A35021) (Table 2C).

TABLE 2C

BLASTX of POLY1 against Myosin-Light-Chain Kinase (EC 2.7.1.117,
Skeletal Muscle-Rabbit) (SEO ID NO:29)

>ptnr:PIR-ID:A35O21 myosin-light-chain kinase (EC 2.7.1.117), skeletal muscle-
rabbit
Top Previous Match Next Match
Length = 608
Plus Strand HSPs:
Score = 2637 (928.3 bits), Expect=2.0e−273, P = 2.0e−273
Identities = 525/608 (86%), Positives = 546/608 (89%), Frame = + 1

| | | | | |
|---|---|---|---|---|
| Query: | 1 | MATENGAVELGIQNPSTDKAPKGPTGERPLAAGKDPGPPDPKKAP---------DPPTLK | 153 | (SEQ ID NO.:2) |
| | | \|\|\|\|\|\|\|\|\|\|\|\|\|+ \|\|\|+\| \|\| \| \|\|\| \|\|\| \|\|\|\|+\| \| \|\| \| \| | | |
| Sbjct: | 1 | MATENGAVELGIQSLSTDEASKGAASEESLAAEKDPAPPDPEKGPGPSDTKQDPDPSTPK | 60 | (SEQ ID NO.:29) |
| Query: | 154 | KDAKAPASEKGDGTLAQPSTS-SQGPKGEGDRGGGPAEGSAGPPAALPQQTATPETSVKK | 330 | |
| | | \|\|\|\|\|\|\|\|\|\|\|\|\|\|\|\|\|\|\|\|+\|\|\|\|\|\|\|\|\|\|\|\|\|\|\|\|\|\|\|\| | | |
| Sbjct: | 61 | KDANTPAPEKGDVVPAQPSAGGSQGPAGEGGQVEAPAEGSAGKPAALPQQTATAEASEKK | 120 | |
| Query: | 331 | PKAEQGASGSQDPGKPRVGKKAAEGQAAARRGSPAFLHSPSCPAIISSSEKLLAKKPPSE | 510 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | PEAEKGPSGHQDPGEPTVGKKVAEGQAAARRGSPAFLHSPSCPAIIASTEKLPAQKPLSE | 180 | |
| Query: | 511 | ASELTFEGVPMTHSPTDPRPAKAEEGKNILAESQKEVGEKTPGQAGQAKMQGDTSRGIEF | 690 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | ASELIFEGVPATPGPTEPGPAKAEGGVDLLAESQKEAGEKAPGQADQAKVQGDTSRGIEF | 240 | |
| Query: | 691 | QAVPSEKS--EVGQALCLTAREEDCFQILDDCPPPPAPFPHRMVELRTGNVSSEFSMNSK | 864 | |
| | | \|\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 241 | QAVPSERPRPEVGQALCLPAREEDCFQILDDCPPPPAPFPHRIVELRTGNVSSEFSMNSK | 300 | |
| Query: | 865 | EALGGGKFGAVCTCMEKATGLKLAAKVIKKQTPKDKEMVLLEIEVMNQLNHRNLIQLYAA | 1044 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | EALGGGKFGAVCTCTEKSTGLKLAAKVIKKQTPKDKEMVMLEIEVMNQLNHRNLIQLYAA | 360 | |
| Query: | 1045 | TETPHEIVLFME-IEGGELFERIVDEDYHLTEVDTMVFVRQICDGILFMHKMRVLHLDLK | 1221 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | IETPHEIVLFMEYIEGGELFERIVDEDYHLTEVDTMVFVRQICDGILFMHKMRVLHLDLK | 420 | |
| Query: | 1222 | PENILCVNTTGHLVKIIDFGLARRYHNPNEKLKVNFGTPEFLSPEVVNYDQISDKTDMWS | 1401 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 421 | PENILCVNTTGHLVKIIDFGLARRY-NPNEKLKVNFGTPEFLSPEVVNYDQTSDKTDMWS | 479 | |
| Query: | 1402 | MGVITYMLLSGLSPFLGDDDTETLNNVLSGNWYFDEETFEAVSDEAKDFVSNLIVKDQ-A | 1578 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 480 | LGVITYMLLSGLSPFLGDDDTETLNNVLSGNWYFDEETFEAVSDEAKDFVSNLIVKEQGA | 539 | |
| Query: | 1579 | RMNAAQCLAHPWLNNLAEKAKRCNRRLKSQILLKKYLMKRRWKKNFIAVSAANRFKKISS | 1758 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 540 | RMSAAQCLAHPWLNNLAEKAKRCNRRLKSQILLKKYLMKRRWKKNFIAVSAANRFKKISS | 599 | |

In a search of sequence databases, it was also found, for example, that the nucleic acid sequence (SEQ ID NO:2) of this invention has 253 of 261 residues (96%) identical to and 258 of 261 residues (98%) similar to a Oryctolagus cuniculus protein kinase #4 polypeptide (PATP Accession No.: AAY43923), is is shown in Table 2D. POLY1 homology with other sequences is shown in Table 2E.

TABLE 2D.

| | | | | |
|---|---|---|---|---|
| Query: | 847 | FSMNSKEALGGGKFGAVCTCMEKATGLKLAAKVIKKQTPKDKEMVLLEIEVMNQLNHRNL | 1026 | (SEQ ID NO.:2) |
| | | \|\|\|\|\|\|\|\|\|\|\|\|\|\|\|\|\|\|\|\|\|\|#\|\|\|\|\|\|\|\|\|\|\|\|\|\|\|\|\|\|\|\|\|#\|\|\|\|\|\|\|\|\|\|\|\|\|\| | | |
| Sbjct: | 1 | FSMNSKEALGGGKFGAVCTCTEKSTGLKLAAKVIKKQTPKDKEMVMLEIEVMNQLNHRNL | 60 | (SEQ ID NO.47) |
| Query: | 1027 | IQLYAAIETPHEIVLFME-IEGGELFERIVDEDYHLTEVDTMVFVRQICDGILFMHKMRV | 1203 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | IQLYAAIETPHEIVLFMEYIEGGELFERIVDEDYHLTEVDTMVFVRQICDGILFMHKMRV | 120 | |
| Query: | 1204 | LHLDLKPENILCVNTTGHLVKIIDFGLARRYHNPNEKLKVNFGTPEFLSPEVVNYDQISD | 1383 | |
| Sbjct: | 121 | LHLDLKPENILCVNTTGHLVKIIDFGLARRY-NFNEKLKVNFGTPEFLSPEVVNYDQISD | 179 | |
| Query: | 1384 | KTDMWSMGVITYMLLSGLSPFLGDDDTETLNNVLSGNWYFDEETFEAVSDEAKDFVSNLI | 1563 | |
| | | \|\|\|\|\|\|#\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 180 | KTDMWSLGVITYMLLSGLSPFLGDDDTETLNNVLSGNWYFDEETFEAVSDEAKDFVSNLI | 239 | |
| Query: | 1564 | VKDQ-ARMNAAQCLAHFWLNNL 1626 | | |
| | | \|\|\|\|\|\|#\|\|\|\|\|\|\|\|\|\|\|\|\| | | |
| Sbjct: | 240 | VKEQGARMSAAQCLAHPWLNNL 261 | | |

TABLE 2E

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAY43923 Rabbit protein kinase #4-Oryctolagus c . . . | +1 | 1305 | 2.5e−132 | 1 |
| patp:AAY42111 Human ischaemic heart disease associated . . . | +1 | 1131 | 6.8e−114 | 1 |
| patp:AAB65634 Novel protein kinase, SEQ ID NO: 161-H . . . | +1 | 1123 | 4.8e−113 | 1 |
| patp:AAB65652 Novel protein kinase, SEQ ID NO: 179-H . . . | +1 | 1042 | 8.6e−107 | 2 |
| patp:AAB42098 Human ORFX ORF1862 polypeptide sequence . . . | +1 | 1028 | 5.6e−103 | 1 |
| patp:AAB56864 Human prostate cancer antigen protein se . . . | +1 | 704 | 4.9e−73 | 2 |

PSORT analysis predicts the protein of the invention to be localized to the nucleus with a certainty of 0.8800. Using the SignalP analysis, it is predicted that the protein of the invention does not have a signal peptide. The predicted molecular weight of a POLY1 polypeptide is 64501.9 daltons.

Quantitative expression of POLY 1 was assessed as described in Example 4.

POLY2

A novel nucleic acid was identified that is comprised of 1788 nucleotides (SEQ ID NO:3) encodes a novel MLCK-like protein that is shown in TABLE 3A. An open reading frame was identified beginning with an ATG initiation codon at nucleotides 1, 2 and 3 and ending with a TGA codon at nucleotides 1786, 1787, 1788. A putative untranslated region downstream from the termination codon is underlined in TABLE 3A, and the start and stop codons are in bold letters. The encoded protein having 595 (SEQ ID NO:4) amino acid residues is presented using the one-letter code in TABLE 3B.

TABLE 3A
Nucleotide sequence of POLY2.

| | |
|---|---|
| >SC87372923-1_EXT | |
| ATGGCGACAGAAAATGGAGCAGTTGAGCTGGGAATTCAGAACCCATCAACAGACAAGGCACCT | (SEQ ID NO:3) |
| AAAGGTCCCACAGGTGAAAGACCCCTGGCTGCAGGGAAAGACCCTGGCCCCCCAGACCCAAAG | |
| AAAGCTCCGGATCCACCCACCCTGAAGAAAGATGCCAAAGCCCCTGCCTCAGAGAAAGGGGAT | |
| GGTACCCTGGCCCAACCCTCAACTAGCAGCCAAGGCCCCAAAGGAGAGGGTGACAGGGGCGGG | |
| GGGCCCGCGGAGGGCAGTGCTGGGCCCCCGGCAGCCCTGCCCCAGCAGACTGCGACACCTGAG | |
| ACCAGCGTCAAGAAGCCCAAGGCTGAGCAGGGAGCCTCAGGCAGCCAGGATCCTGGAAAGCCC | |
| AGGGTGGGCAAGAAGGCAGCAGAGGGCCAAGCAGCAGCCAGGAGGGGCTCACCTGCCTTTCTG | |
| CATAGCCCCAGCTGTCCTGCCATCATCTCCAGTTCTGAGAAGCTGCTGGCCAAGAAGCCCCCAA | |
| GCGAGGCATCAGAGCTCACCTTTGAAGGGGTGCCCATGACCCACAGCCCCACGGATCCCAGGC | |
| CAGCCAAGGCAGAAGAAGGAAAGAACATCCTGGCAGAGAGCCAGAAGGAAGTGGGAGAGAA | |
| AACCCCAGGCCAGGCTGGCCAGGCTAAGATGCAAGGGGACACCTCGAGGGGGATTGATTCCA | |
| GGCTGTTCCCTCAGAGAAATCCGAGGTGGGGCAGGCCCTCTGTCTCACAGCCAGGGAGGAGGA | |
| CTGCTTCCAGATTTTGGATGATTGCCCGCCACCTCCGGCCCCCTTCCCTCACCGCATGGTGGAGC | |
| TGAGGACCGGGAATGTCAGCAGTGAAATTCAGTATGAACTCCAAGGAGGCGCTCGGAGGGGGCA | |
| AGTTTGGGGCAGTCTGTACCTGCATGGAGAAAGCCACAGGCCTCAAGCTGGCAGCCAGGTCA | |
| TCAAGAAACAGACTCCCAAAGACAAGGAAATGGTGTTGCTGGAGATTGAGGTCATGAACCAGC | |
| TGAACCACCGCAATCTGATCCAGCTGTATGCAGCCATCGAGACTCCGCATGAGATCGTCCTGTT | |
| CATGGAGATCGAGGGCGGAGAGCTCTTCGAGAGGATTGTGGATGAGGACTACCATCTGACCGA | |
| GGTGGACACCATGGTGTTTGTCAGGCAGATCTGTGACGGGATCCTCTTGATGCACAAGATGAGG | |
| GTTTTGCACCTGGACCTCAAGCCAGAGAACATCCTGTGTGTCAACACCACCGGGCATTTGGTGA | |
| AGATCATTGACTTTGGCCTGGCACGGAGGTATAACCCCAACGAGAAGCTGAAGGTGAACTTTG | |
| GGACCCCAGAGTTCCTGTCACCTGAGGTGGTGAATTATGACCAAATCTCCGATAAGACAGACAT | |
| GTGGAGTATGGGGGTGATCACCTACATGCTGCTGAGCGGCCTCTCCCCCTTCCTGGGAGATGAT | |
| GACACAGAGACCCTAAACAACGTTCTATCTGGCAACTGGTACTTTGATGAAGAGACCTTTGAGG | |
| CCGTATCAGACGAGGCCAAAGACTTTGTCTCCAACCTCATCGTCAAGGACCAGAGGGCCCGGA | |
| TGAACGCTGCCCAGTGTCTCGCCCATCCCTGGCTCAACAACCTGGCGGAGAAAGCCAAACGCTG | |
| TAACCGACGCCTTAAGTCCCAGATCTTGCTTAAGAAATACCTCATGAAGAGGCGCTGGAAGAA | |
| AAACTTCATTGCTGTCAGCGCTGCCAACCGCTTCAAGAAGATCAGCAGCTCGGGGGCACTGATG | |
| GCTCTGGGGGTCTGAGCCCTGGGCGCAGCTGAAGCCTGGACGCAGCCACACAGTGGCCGGGGC | |
| TGAAGCCACACAGCCCAGAAGGCCAGAAAAGGCAGCCAGATCCCCAGGGCAGCCTCTTAGGA | |
| CAAGGCTGTGCCAGGCTGGGAGGCTCGGGGCTCCCCACGCCCCCATGCAGTGACCGCTTCCCCG | |
| ATGTGAGC | |

TABLE 3B
Protein sequence encoded by the coding sequence shown in TABLE 3A

Frame: +1—-Nucleotide 1 to 1785-595 amino acid reading frame-

(SEQ ID NO:4)

| | |
|---|---|
| MATENGAVELGIQNPSTDKAPKGPTGERPLAAGKDPGPPDPKKAPDPPTLKKDAKAPASEKGDGTLAQP | +TL,45 |
| STSSQGPKGEGDRGGGPAEGSAGPPAALPQQTATPETSVKKPKAEQGASGSQDPGKPRVGKKAAEGQA | |
| AARRGSPAFLHSPSCPAIISSSEKLLAKKPPSEASELTFEGVPMTHSPTDPRPAKAEEGKNILAESQKEVGE | |
| KTPGQAGQAKMQGDTSRGIEFQAVPSEKSEVGQALCLTAREEDCFQILDDCPPPPAPFPHRMVELRTGN | |
| VSSEFSMNSKEALGGGKFGAVCTCMEKATGLKLAAKVIKKQTPKDKEMVLLEIEVMNQLNHRNLIQLY | |
| AAIETPHEIVLFMEIEGGELFERIVDEDYHLTEVDTMVFVRQICDGILLMHKMRVLHLDLKPENILCVNTT | |
| GHLVKIIDFGLARRYNPNEKLKVNFGTPEFLSPEVVNYDQISDKTDMWSMGVITYMLLSGLSPFLGDDD | |
| TETLNNVLSGNWYFDEETFEAVSDEAKDFVSNLIVKDQRARMNAAQCLAHPWLNNLAEKAKRCNRRL | |
| KSQILLKKYLMKRRWKKNFIAVSAANRFKKISSSGALMALGV | |

In a search of sequence databases, it was found, for example, that the nucleic acid sequence (SEQ ID NO: 3) has 1461 of 1767 bases (82%) identical to a Rattus norvegicus Skeletal Muscle Light Chain Kinase mRNA (GENBANK-ID: RATMLCK|acc:J03886). The amino acid sequence of the protein of the invention was found to have 524 of 608 amino acid residues (86%) identical to, and 545 of 608 residues (89%) similar to, the 608 amino acid residue MLCK protein from O. cuniculus (ptnr: PIR-ID: A35021) (Table 3C).

TABLE 3C

BLASTX of POLY2 against Myosin Light Chain Kinase (EC 2.7.1.117),
Skeletal Muscle-Rabbit (SEQ ID NO:30)

>ptnr:PIR-ID:A35021 myosin-light-chain kinase (EC 2.7.1.117), skeletal muscle-
rabbit
Top Previous Match Next Match
Length = 608
Plus Strand HSPs:
Score = 2647 (931.8 bits), Expect = 1.6e−274, P=1.6e−274
Identities = 524/608 (86%), Positives = 545/608 (89%), Frame = +1

| | | | | |
|---|---|---|---|---|
| Query: | 1 | MATENGAVELGIQNPSTDKAPKGPTGERPLAAGKDPGPPDPKKAP---------DPPTLK | 153 | (SEQ ID NO.:4) |
| | | \|\|\|\|\|\|\|\|\|\|\|\|\|+\|\|\|#\|\|\|\|\|\|\|\|\|\|\|\|\|\|#\|\| \|\|\|\| | | |
| Sbjct: | 1 | MATENGAVELGIQSLSTDEASKGAASEESLAAEKDPAPPDPEKGPGPSDTKQDPDPSTPK | 60 | (SEQ ID NO:30) |
| Query: | 154 | KDAKAPASEKGDGTLAQPSTS-SQGPKGEGDRGGGPAEGSAGPPAALPQQTATPETSVKK | 330 | |
| | | \|\|\|\|\|\|\|\|\|\|\|\|\|\|\|\|\|\|\|\|+\|\|\|\|\|\|\|\|\|\|\|\|\|\|\|\|\|\|\|\|\| | | |
| Sbjct: | 61 | KDANTPAPEKGDVVPAQPSAGGSQGPAGEGGQVEAPAEGSAGKPAALPQQTATAEASEKK | 120 | |
| Query: | 331 | PKAEQGASGSQDPGKPRVGKKAAEGQAAARRGSPAFLHSPSCPAIISSSEKLLAKKPPSE | 510 | |
| | | \|#\|\|#\|\|\|\|\|\|\|#\|\|\|\|\|\|\|\|\|\|\|\|\|\|\|\|\|\|\|\|\|\|\|\|\|\|\|\|\|#\|#\|\|\|\|#\|\|\|\| | | |
| Sbjct: | 121 | PEAEKGPSGHQDPGEPTVGKKVAEGQAAARRGSPAFLHSPSCPAIIASTEKLPAQKPLSE | 180 | |
| Query: | 511 | ASELTFEGVPMTHSPTDPRPAKAEEGKNILAESQKEVGEKTPGQAGQAKMQGDTSRGIEF | 690 | |
| | | \|\|\|\|\|\|\|\|\|\|\|\|#\|\|\|\|\|\|\|##\|\|\|\|\|\|\|\|\|\|\|\|\|\|\|\|\|#\|\|\|\|\|\|\|\|\|\| | | |
| Sbjct: | 181 | ASELIFEGVPATPGPTEPGPAKAEGGVDLLAESQKEAGEKAPGQADQAKVQGDTSRGIEF | 240 | |
| Query: | 691 | QAVPSEKS--EVGQALCLTAREEDCFQILDDCPPPPAPFPHRMVELRTGNVSSEFSMNSK | 864 | |
| | | \|\|\|\|\|\|+\|\|\|\|\|\|\|\|\|\|\|\|\|\|\|\|\|\|\|\|\|\|\|\|\|\|\|\|\|\|\|#\|\|\|\|\|\|\|\|\|\|\|\|\|\|\|\|\| | | |
| Sbjct: | 241 | QAVPSERPRPEVGQALCLPAREEDCFQILDDCPPPPAPFPHRIVELRTGNVSSEFSMNSK | 300 | |
| Query: | 865 | EALGGGKFGAVCTCMEKATGLKLAAKVIKKQTPKDKEMVLLEIEVMNQLNHRNLIQLYAA | 1044 | |
| | | \|\|\|\|\|\|\|\|\|\|\|\|\|\|\|\|#\|\|\|\|\|\|\|\|\|\|\|\|\|\|\|\|\|\|\|\|\|#\|\|\|\|\|\|\|\|\|\|\|\|\|\|\|\|\|\|\|\| | | |
| Sbjct: | 301 | EALGGGKFGAVCTCTEKSTGLKLAAKVIKKQTPKDKEMVMLEIEVMNQLNHRNLIQLYAA | 360 | |
| Query: | 1045 | IETPHEIVLFME-IEGGELFERIVDEDYHLTEVDTMVFVRQICDGILLMHKMRVLHLDLK | 1221 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | IETPHEIVLFMEYIEGGELFERIVDEDYHLTEVDTMVFVRQICDGILFMHKMRVLHLDLK | 420 | |
| Query: | 1222 | PENILCVNTTGHLVKIIDFGLARRYNPNEKLKVNFGTPEFLSPEVVNYDQISDKTDWSM | 1401 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|# | | |
| Sbjct: | 421 | PENILCVNTTGHLVKIIDFGLARRYNPNEKLKVNFGTPEFLSPEVVNYDQISDKTDMWSL | 480 | |
| Query: | 1402 | GVITYMLLSGLSPFLGDDDTETLNNVLSGNWYFDEETFEAVSDEAKDFVSNLIVKDQRAR | 1581 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|#\|\|\| | | |
| Sbjct: | 481 | GVITYMLLSGLSPFLGDDDTETLNNVLSGNWYFDEETFEAVSDEAKDFVSNLIVKEQGAR | 540 | |
| Query: | 1582 | MNAAQCLAHPWLNNLAEKAKRCNRRLKSQILLKKYLMKRRWKKNFIAVSAANRFKKISSS | 1761 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 541 | MSAAQCLAHPWLNNLAEKAKRCNRRLKSQILLKKYLMKRRWKKNFIAVSAANRFKKISSS | 600 | |

PSORT analysis predicts the protein of the invention to be localized in the nucleus. Using the SignalP analysis, it is predicted that the protein of the invention does not have a signal peptide.

POLY3

A POLY3 nucleic acid was identified as described in Example 3 that is comprised of 2558 nucleotides (SEQ ID NO: 5) encoding a novel MLCK-like protein that is shown in Table 4A. An open reading frame was identified beginning with an ATG initiation codon at nucleotides 164-166 and ending with a TGA codon at nucleotides 1949-1951. A putative untranslated region downstream from the termination codon is underlined in Table 4A, and the start and stop codons are in bold letters. The encoded protein having 595 (SEQ ID NO:6) amino acid residues is presented using the one-letter code in Table 4B.

TABLE 4A
Nucleotide sequence of POLY3.

| | | |
|---|---|---|
| >CG51448-04 | | |
| CTTTGCTCCAGGTACCTCTCTCCCCTCAGTTAGCAGGCCTCGGCTTCCTGTCTCACTGCA | 60 | (SEQ ID NO:5) |
| GCCAGACGAGAGGGGAAATTGGACAGCCTGACACACTCCACTCTTGTTTCTGCAGCTAGA | 120 | |
| AAGACTTGAGTTAGACAAGCAGCAGCACACGCCTCCCTACCTCATGGCGACAGAAAATGG | 180 | |
| AGCAGTTGAGCTGGGAATTCAGAACCCATCAACAGACAAGGCACCTAAAGCGTCCCACAGG | 240 | |
| TGAAAGACCCCTGGCTGCAGGGAAAGACCCTGGCCCCCCAGACCCAAAGAAAGCTCCGGA | 300 | |
| TCCACCCACCCTGAAGAAAGATGCCAAAGCCCCTGCCTCAGAGAAAGGGGATGGTACCCT | 360 | |
| GGCCCAACCCTCAACTAGCAGCCAAGGCCCCAAAGGAGAGGGTGACAGGGGCGGGGGGCC | 420 | |
| CGCGGAGGGCAGTGCTGGGCCCCCGGCAGCCCTGCCCCAGCAGACTGCGACACCTGAGAC | 480 | |
| CAGCGTCAAGAAGCCCAAGGCTGAGCAGGGAGCCTCAGGCAGCCAGGATCCTGGAAAGCC | 540 | |
| CAGGGTGGGCAAGAAGGCAGCAGAGGGCCAAGCAGCAGCCAGGAGGGGCTCACCTGCCTT | 600 | |
| TCTGCATAGCCCCAGCTGTCCCGCCATCATCTCCAGTTCTGAGAAGCTGCTGGCCAAGAA | 660 | |
| GCCCCCAAGCGAGGCATCAGAGCTCACCTTTGAAGGGGTGCCCATGACCCACAGCCCCAC | 720 | |
| GGATCCCAGGTCGGCCAAGGCAGAAGAAGGAAAGAACATCCTGGCAGAGAGCCAGAAGGA | 780 | |
| AGTGGGAGAGAAAACCCCAGGCCAGGCTGGCCAGGCTAAGATGCAAGGGGACACCTCGAG | 840 | |
| GGGGATTGAGTTCCAGGCTGTTCCCTCAGAGAAATCCGAGGTGGGGCAGGCCCTCTGTCT | 900 | |
| CACAGCCAGGGAGGAGGACTGCTTCCAGATTTTGGATGATTGCCCGCCACCTCCGGCCCC | 960 | |
| CTTCCCTCACCGCATGGTGGAGCTGAGGACCGGGAATGTCAGCAGTGAATTCAGTATGAA | 1020 | |
| CTCCAAGGAGGCGCTCGGAGGGGGCAAGTTTGGGGCAGTCTGTACCTGCATGGAGAAAGC | 1080 | |
| CACAGGCCTCAAGCTGGCAGCCAAGGTCATCAAGAAACAGACTCCCAAAGACAAGGAAAT | 1140 | |
| GGTGTTGCTGGAGATTGAGGTCATGAACCAGCTGAACCACCGCAATCTGATCCAGCTGTA | 1200 | |
| TGCAGCCATCGAGACTCCGCATGAGATCGTCCTGTTCATGGAGATCGAGGGCGGAGAGCT | 1260 | |
| CTTCGAGAGGATTGTGGATGAGGACTACCATCTGACCGAGGTGGACACCATGGTGTTTGT | 1320 | |
| CAGGCAGATCTGTGACGGGATCCTCTTGATGCACAAGATGAGGGTTTTGCACCTGGACCT | 1380 | |
| CAAGCCAGAGAACATCCTGTGTGTCAACACCACCGGGCATTTGGTGAAGATCATTGACTT | 1440 | |
| TGGCCTGGCACGGAGGTATAACCCCAACGAGAAGCTGAAGGTGAACTTTGGGACCCCAGA | 1500 | |
| GTTCCTGTCACCTGAGGTGGTGAATTATGACCAAATCTCCGATAAGACAGACATGTGGAG | 1560 | |
| TATGGGGGTGATCACCTACATGCTGCTGAGCGGCCTCTCCCCCTTCCTGGGAGATGATGA | 1620 | |
| CACAGAGACCCTAAACAACGTTCTATCTGGCAACTGGTACTTTGATGAAGAGACCTTTGA | 1680 | |
| GGCCGTATCAGACGAGGCCAAAGACTTTGTCTCCAACCTCATCGTCAAGGACCAGAGGGC | 1740 | |
| CCGGATGAACGCTGCCCAGTGTCTCGCCCATCCCTGGCTCAACAACCTGGCGGAGAAAGC | 1800 | |
| CAAACGCTGTAACCGACGCCTTAAGTCCCAGATCTTGCTTAAGAAATACCTCATGAAGAG | 1860 | |
| GCGCTGGAAGAAAAACTTCATTGCTGTCAGCGCTGCCAACCGCTTCAAGAAGATCAGCAG | 1920 | |
| CTCGGGGGCACTGATGGCTCTGGGGGTCTGAGCCCTGGGCGCAGCTCAAGCCTGGACGCA | 1980 | |
| GCCACACAGTGGCCGGGGCTGAAGCCACACAGCCCAGAAGGCCAGAAAAGGCAGCCAGAT | 2040 | |
| CCCCAGGGCAGCCTCGTTAGGACAAGGCTGTGCCAGGCTGGGAGGCTCGGGGCTCCCCAC | 2100 | |
| GCCCCCATGCAGTGACCGCTTCCCCGATGTGAGCCGCCTCGGAGTGTGGCCTGGATCCAT | 2160 | |
| CCTGCTAGCACCTCCCCAGACAGGGCTCCAGCCTGTCGGCCACACCCCAGACTCCAGGCC | 2220 | |
| CCCGTTGAAGCCGCTCCCGGTTCCCTCCCCAGCTCCTCGTCTTTGAACTGCCGCCGCCGT | 2280 | |
| GGTGACCCCTGCTTTGCCCCACTGGGAGAGTCCTTAGCCTGGGCCTCCTCCTACCTCCAG | 2340 | |
| TGCCATGGCTGGGGGGTCTCAGCATGTAGGGCTTCTGTGGTTGTGGATGGGAGGCTCCTG | 2400 | |
| GTGGGGCAGAAAGGCTGCAACGCTGATTCCTAAGGCCCAGCTGCCAGGGAAGACAGAGCA | 2460 | |
| GGCTTTGTGAGAGAGGACCTCCATGCCCCCGCCACCTCCCCACTCCAGCAGATAAGGCCG | 2520 | |
| AGCCCACACCATCTGGCCCAGGCTGGCCCCCACCACCT 2558 | | |

TABLE 4B
Protein sequence encoded by the coding sequence shown in TABLE 4A

>CG51448-04

| | | |
|---|---|---|
| MATENGAVELGIQNPSTDKAPKGPTGERPLAAGKDPGPPDPKKAPDPPTLKKDAKAPASE | 60 | (SEQ ID NO:6) |
| KGDGTLAQPSTSSQGPKGEGDRGGGPAEGSAGPPAALPQQTATPETSVKKPKAEQGASGS | 120 | |
| QDPGKPRVGKKAAEGQAAARRGSPAFLHSPSCPAIISSSEKLLAKKPPSEASELTFEGVP | 180 | |
| MTHSPTDPRSAKAEEGKNILAESQKEVGEKTPGQAGQAKMQGDTSRGIEFQAVPSEKSEV | 240 | |
| GQALCLTAREEDCFQILDDCPPPPAPFPHRMVELRTGNVSSEFSMNSKEALGGGKFGAVC | 300 | |
| TCMEKATGLKLAAKVIKKQTPKDKEMVLLEIEVMNQLNHRNLIQLYAAIETPHEIVLFME | 360 | |
| IEGGELFERIVDEDYHLTEVDTMVFVRQICDGILLMHKMRVLHLDLKPENILCVNTTGHL | 420 | |
| VKIIDFGLARRYNPNEKLKVNFGTPEFLSPEVVNYDQISDKTDMWSMGVITYMLLSGLSP | 480 | |
| FLGDDDTETLNNVLSGNWYFDEETFEAVSDEAKDFVSNLIVKDQRARMNAAQCLAHPWLN | 540 | |
| NLAEKAKRCNRRLKSQILLKKYLMKRRWKKNFIAVSAANRFKKISSSGALMALGV | 595 | |

In a search of sequence databases, it was found, for example, that the nucleic acid sequence (SEQ ID NO: 5) has 1360 of 1534 bases (88%) identical to a gb:GENBANK-ID:RABMLCKA|acc:J05194.1 mRNA from Oryctolagus cuniculus (Rabbit myosin light chain kinase mRNA, complete cds). The full amino acid sequence of the protein of the invention was found to have 593 of 596 amino acid residues (99%) identical to, and 593 of 596 amino acid residues (99%) similar to, the 596 amino acid residue ptnr:TREMBLNEW-ACC:AAK15494 protein from Homo sapiens (SKELETAL MYOSIN LIGHT CHAIN KINASE) (Table 4C).

TABLE 4C
BLASTP of POLY3 against SKELETAL MYOSIN LIGHT CHAIN
KINASE-Homo sapiens (SEQ ID NO:31)

```
BLASTP search using the protein of CuraGen Acc. No. CG51448-04.
>ptnr:TREMBLNEW-ACC:AAK15494 SKELETAL MYOSIN LIGHT
CHAIN KINASE-Homo sapiens
                      (Human), 596 aa.
                      Length = 596
Score = 3076 (1082.8 bits), Expect = 0.0, P = 0.0
Identities = 593/596 (99%), Positives = 593/596 (99%)
```

| | | | | |
|---|---|---|---|---|
| Query: | 1 | MATENGAVELGIQNPSTDKAPKGPTGERPLAAGKDPGPPDPKKAPDPPTLKKDAKAPASE | 60 | (SEQ ID NO.:6) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | MATENGAVELGIQNPSTDKAPKGPTGERPLAAGKDPGPPDPKKAPDPPTLKKDAKAPASE | 60 | (SEQ ID NO:31) |
| Query: | 61 | KGDGTLAQPSTSSQGPKGEGDRGGGPAEGSAGPPAALPQQTATPETSVKKPKAEQGASGS | 120 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | KGDGTLAQPSTSSQGPKGEGDRGGGPAEGSAGPPAALPQQTATPETSVKKPKAEQGASGS | 120 | |
| Query: | 121 | QDPGKPRVGKKAAEGQAAARRGSPAFLHSPSCPAIISSSEKLLAKKPPSEASELTFEGVP | 180 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | QDPGKPRVGKKAAEGQAAARRGSPAFLHSPSCPAIISSSEKLLAKKPPSEASELTFEGVP | 180 | |
| Query: | 181 | MTHSPTDPRSAKAEEGKNILAESQKEVGEKTPGQAGQAKMQGDTSRGIEFQAVPSEKSEV | 240 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | MTHSPTDPRPAKAEEGKNILAESQKEVGEKTPGQAGQAKMQGDTSRGIEFQAVPSEKSEV | 240 | |
| Query: | 241 | GQALCLTAREEDCFQILDDCPPPPAPFPHRMVELRTGNVSSEFSMNSKEALGGGKFGAVC | 300 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 241 | GQALCLTAREEDCFQILDDCPPPPAPFPHRMVELRTGNVSSEFSMNSKEALGGGKFGAVC | 300 | |
| Query: | 301 | TCMEKATGLKLAAKVIKKQTPKDKEMVLLEIEVMNQLNHRNLIQLYAAIETPHEIVLFME | 360 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | TCMEKATGLKLAAKVIKKQTPKDKEMVLLEIEVMNQLNHRNLIQLYAAIETPHEIVLFME | 360 | |
| Query: | 361 | -IEGGELFERIVDEDYHLTEVDTMVFVRQICDGILLMHKMRVLHLDLKPENILCVNTTGH | 419 | |
| | | \|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\| | | |
| Sbjct: | 361 | YIEGGELFERIVDEDYHLTEVDTMVFVRQICDGILFMHKMRVLHLDLKPENILCVNTTGH | 420 | |
| Query: | 420 | LVKIIDFGLARRYNPNEKLKVNFGTPEFLSPEVVNYDQISDKTDMWSMGVITYMLLSGLS | 479 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 421 | LVKIIDFGLARRYNPNEKLKVNFGTPEFLSPEVVNYDQISDKTDMWSMGVITYMLLSGLS | 480 | |
| Query: | 480 | PFLGDDDTETLNNVLSGNWYFDEETFEAVSDEAKDFVSNLIVKDQRARMNAAQCLAHPWL | 539 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 481 | PFLGDDDTETLNNVLSGNWYFDEETFEAVSDEAKDFVSNLIVKDQRARMNAAQCLAHPWL | 540 | |
| Query: | 540 | NNLAEKAKRCNRRLKSQILLKKYLMKRRWKKNFIAVSAANRFKKISSSGALMALGV | 595 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 541 | NNLAEKAKRCNRRLKSQILLKKYLMKRRWKKNFIAVSAANRFKKISSSGALMALGV | 596 | |

PSORT analysis predicts the protein of the invention to be localized in the nucleus with a certainty of 0.8800. Using the SignalP analysis, it is predicted that the protein of the invention does not have a signal peptide.

SNPs and cSNPs:

Single nucleotide polymorphism analysis is detailed in Example 2. As is shown in Table 4D, in the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 4D
Cons.Pos.: 515 Depth: 30 Change: C > T Putative Allele Freq.: 0.067
Cons.Pos.: 728 Depth: 17 Change: G > A Putative Allele Freq.: 0.118
Cons.Pos.:2303 Depth: 36 Change: T > C Putative Allele Freq.: 0.056
Cons.Pos.:2459 Depth: 18 Change: G > C Putative Allele Freq.: 0.111

POLY4

A novel nucleic acid was identified that is comprised of 1839 nucleotides (SEQ ID NO: 7), which encodes a MLCK-like protein is shown in Table 5A. An open reading frame was identified beginning with an ATG initiation codon at nucleotides 49-51 and ending with a TGA codon at nucleotides 1837-1839. The start and stop codons are in bold letters. Putative untranslated regions, if any, are found upstream from the initiation codon and downstream from the termination codon. The encoded protein having 596 amino acid residues (SEQ ID NO: 8) is presented using the one-letter code in Table 5B.

TABLE 5A
The nucleotide sequence of POLY 4

>153574419 tpn REVCOMP

| | | |
|---|---|---|
| CTAGAAGACTTGAGTTAGACAAGCAGCACCACACGCCTCCCTACCTCATGGCGACAGAA | 60 | (SEQ ID NO:7) |
| AATGGAGCAGTTGAGCTGGGAATTCAGAACCCATCAACAGACAAGGCACCTAAAGGTCCC | 120 | |
| ACAGGTGAAAGACCCCTGGCTGCAGGGAAAGACCCTGGCCCCCCAGACCCAAAGAAAGCT | 180 | |
| CCGGATCCACCCACCCTGAAGAAAGATGCCAAAGCCCCTGCCTCAGAGAAAGGGGATGGT | 240 | |
| ACCCTGGCCCAACCCTCAACTAGCAGCCAAGGCCCCAAAGGAGAGGGTGACAGGGGCGGG | 300 | |
| GGGCCCGCGGAGGGCAGTGCTGGGCCCCCGGCAGCCCTGCCCCAGCAGACTGCGACACCT | 360 | |
| GAGACCAGCGTCAAGAAGCCCAAGGCTGAGCAGGGAGCCTCAGGCAGCCAGGATCCTGGA | 420 | |
| AAGCCCAGGGTGGGCAAGAAGGCAGCAGAGGGCCAAGCAGCAGCCAGGAGGGGCTCACCT | 480 | |
| GCCTTTCTGCATAGCCCCAGCTGTCCTGCCATCATCTCCAGTTCTGAGAAGCTGCTGGCC | 540 | |
| AAGAAGCCCCCAAGCGAGGCATCAGAGCTCACCTTTGAAGGGGTGCCCATGACCCACAGC | 600 | |
| CCCACGGATCCCAGGCCAGCCAAGGCAGAAGAAGGAAAGAACATCCTGGCAGAGAGCCAG | 660 | |
| AAGGAAGTGGGAGAGAAAACCCCAGGCCAGGCTGGCCAGGCTAAGATGCAAGGGGACACC | 720 | |
| TCGAGGGGGATTGAGTTCCAGGCTGTTCCCTCAGAGAAATCCGAGGTGGGGCAGGCCCTC | 780 | |
| TGTCTCACAGCCAGGGAGGAGGACTGCTTCCAGATTTTGGATGATTGCCCGCCACCTCCG | 840 | |
| GCCCCCTTCCCTCACCGCATGGTGGAGCTGAGGACCGGGAATGTCAGCAGTGAATTCAGT | 900 | |
| ATGAACTCCAAGGAGGCGCTCGGAGGTGGCAAGTTTGGGGCAGTCTGTACCTGCATGGAG | 960 | |
| AAAGCCACAGGCCTCAAGCTGGCAGCCAAGGTCATCAAGAAACAGACTCCCAAAGACAAG | 1020 | |
| GAAATGGTGTTGCTGGAGATTGAGGTCATGAACCAGCTGAACCACCGCAATCTGATCCAG | 1080 | |
| CTGTATGCAGCCATCGAGACTCCGCATGAGATCGTCCTGTTCATGGAGTACATCGAGGGC | 1140 | |
| GGAGAGCTCTTCGAGAGGATTGTGGATGAGGACTACCATCTGACCGAGGTGGACACCATG | 1200 | |
| GTGTTTGTCAGGCAGATCTGTGACGGGATCCTCTTCATGCACAAGATGAGGGTTTTGCAC | 1260 | |
| CTGGACCTCAAGCCAGAGAACATCCTGTGTGTCAACACCACCGGGCATTTGGTGAAGATC | 1320 | |
| ATTGACTTTGGCCTGGCACGGAGGTATAACCCCAACGAGAAGCTGAAGGTGAACTTTGGG | 1380 | |
| ACCCCAGAGTTCCTGTCACCTGAGGCGGTGAATTATGACCAAATCTCCGATAAGACAGAC | 1440 | |
| ATGTGGAGTATGGGGGTGATCACCTACATGCTGCTGAGCGGCCTCTCCCCCTTCCTGGGA | 1500 | |
| GATGATGACACAGAGACCCTAAACAACGTTCTATCTGGCAACTGGTACTTTGATGAAGAG | 1560 | |
| ACCTTTGAGGCCGTATCAGACGAGGCCAAAGACTTTGTCTCCAACCTCATCGTCAAGGAC | 1620 | |
| CAGAGGGCCCGGATGAACGCTGCCCAGTGTCTCGCCCATCCCTGGCTCAACAACCTGGCG | 1680 | |
| GAGAAAGCCAAACGCTGTAACCGACGCCTTAAGTCCCAGATCTTGCTTAAGAAATACCTC | 1740 | |
| ATGAAGAGGCGCTGGAAGAAAAACTTCATTGCTGTCAGCGCTGCCAACCGCTTCAAGAAG | 1800 | |
| ATCAGCAGCTCGGGGGCACTGATGGCTCTGGGGGTCTGA | 1839 | |

TABLE 5B
Protein sequence encoded by the coding sequence shown in TABLE 5A

>153574419_tpn_REVCOMP

| | | |
|---|---|---|
| MATENGAVELGIQNPSTDKAPKGPTGERPLAAGKDPGPPDPKKAPDPPTLKKDAKAPASE | 60 | (SEQ ID NO:8) |
| KGDGTLAQPSTSSQGPKGEGDRGGGPAEGSAGPPAALPQQTATPETSVKKPKAEQGASGS | 120 | |
| QDPGKPRVGKKAAEGQAAARRGSPAFLHSPSCPAIISSSEKLLAKKPPSEASELTFEGVP | 180 | |
| MTHSPTDPRPAKAEEGKNILAESQKEVGEKTPGQAGQAKMQGDTSRGIEFQAVPSEKSEV | 240 | |
| GQALCLTAREEDCFQILDDCPPPPAPFPHRMVELRTGNVSSEFSMNSKEALGGGKFGAVC | 300 | |
| TCMEKATGLKLAAKVIKKQTPKDKEMVLLEIEVMNQLNHRNLIQLYAAIETPHEIVLFME | 360 | |
| YIEGGELFERIVDEDYHLTEVDTMVFVRQICDGILFMHKMRVLHLDLKPENILCVNTTGH | 420 | |
| LVKIIDFGLARRYNPNEKLKVNFGTPEFLSPEAVNYDQISDKTDMWSMGVITYMLLSGLS | 480 | |
| PFLGDDDTETLNNVLSGNWYFDEETFEAVSDEAKDFVSNLIVKDQRARMNAAQCLAHPWL | 540 | |
| NNLAEKAKRCNRRLKSQILLKKYLMKRRWKKNFIAVSAANRFKKISSSGALMALGV | 596 | |

In a search of sequence databases, it was found, for example, that the nucleic acid sequence (SEQ ID NO:7) of this invention has 1441 of 1626 bases (88%) identical to a gb:GENBANK-ID:RABMLCK|acc:J01594.1 mRNA from Oryctolagus cuniculus (rabbit myosin light chain kinase mRNA). The full amino acid sequence of the protein of the invention was found to have 595 of 596 amino acid residues (99%) identical to, and 595 of 596 residues (99%) similar to, the 596 amino acid residue ptnr:TREMBLNEW-ACC:CAC10006 protein from Homo sapiens (Human) (BA243J16.3 (SIMILAR TO MYLK (MYOSIN, LIGHT POLYPEPTIDE KINASE) (Table 5C).

TABLE 5C
BlastP against Similar to MYLK-Homo Sapiens (Human) (SEQ ID NO:32)

```
>ptnr:TREMBLNEW-ACC:CACl0006 BA243J16.3 (SIMILAR TO MYLK (MYOSIN, LIGHT
                        POLYPEPTIDE KINASE)) - Homo sapiens (Human), 596 aa.
                        Length = 596
Score = 3102 (1092.0 bits), Expect = 0.0, P = 0.0
Identities = 595/596 (99%), Positives = 595/596 (99%)
```

| | | | | |
|---|---|---|---|---|
| Query: | 1 | MATENGAVELGIQNPSTDKAPKGPTGERPLAAGKDPGPPDPKKAPDPPTLKKDAKAPASE | 60 | (SEQ ID NO.:8) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | MATENGAVELGIQNPSTDKAPKGPTGERPLAAGKDPGPPDPKKAPDPPTLKKDAKAPASE | 60 | (SEQ ID NO:32) |
| Query: | 61 | KGDGTLAQPSTSSQGPKGEGDRGGGPAEGSAGPPAALPQQTATPETSVKKPKAEQGASGS | 120 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | KGDGTLAQPSTSSQGPKGEGDRGGGPAEGSAGPPAALPQQTATPETSVKKPKAEQGASGS | 120 | |
| Query: | 121 | QDPGKPRVGKKAAEGQAAARRGSPAFLHSPSCPAIISSSEKLLAKKPPSEASELTFEGVP | 180 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | QDPGKPRVGKKAAEGQAAARRGSPAFLHSPSCPAIISSSEKLLAKKPPSEASELTFEGVP | 180 | |
| Query: | 181 | MTHSPTDPRPAKAEEGKNILAESQKEVGEKTPGQAGQAKMQGDTSRGIEFQAVPSEKSEV | 240 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | MTHSPTDPRPAKAEEGKNILAESQKEVGEKTPGQAGQAKMQGDTSRGIEFQAVPSEKSEV | 240 | |
| Query: | 241 | GQALCLTAREEDCFQILDDCPPPPAPFPHRMVELRTGNVSSEFSMNSKEALGGGKFGAVC | 300 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 241 | GQALCLTAREEDCFQILDDCPPPPAPFPHRMVELRTGNVSSEFSMNSKEALGGGKFGAVC | 300 | |
| Query: | 301 | TCMEKATGLKLAAKVIKKQTPKDKEMVLLEIEVMNQLNHRNLIQLYAAIETPHEIVLFME | 360 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | TCMEKATGLKLAAKVIKKQTPKDKEMVLLEIEVMNQLNHRNLIQLYAAIETPHEIVLFME | 360 | |
| Query: | 361 | YIEGGELFERIVDEDYHLTEVDTMVFVRQICDGILFMHKMRVLHLDLKPENILCVNTTGH | 420 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | YIEGGELFERIVDEDYHLTEVDTMVFVRQICDGILFMHKMRVLHLDLKPENILCVNTTGH | 420 | |
| Query: | 421 | LVKIIDFGLARRYNPNEKLKVNFGTPEFLSPEAVNYDQISDKTDMWSMGVITYMLLSGLS | 480 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 421 | LVKIIDFGLARRYNPNEKLKVNFGTPEFLSPEVVNYDQISDKTDMWSMGVITYMLLSGLS | 480 | |
| Query: | 481 | PFLGDDDTETLNNVLSGNWYFDEETFEAVSDEAKDFVSNLIVKDQRARMNAAQCLAHPWL | 540 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Subjct: | 481 | PFLGDDDTETLNNVLSGNWYFDEETFEAVSDEAKDFVSNLIVKDQRARMNAAQCLAHPWL | 540 | |
| Query: | 541 | NNLAEKAKRCNRRLKSQILLKKYLMKRRWKKNFIAVSAANRFKKISSSGALMALGV | 596 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 541 | NNLAEKAKRCNRRLKSQILLKKYLMKRRWKKNFIAVSAANRFKKISSSGALMALGV | 596 | |

PSORT analysis predicts the protein of the invention to be localized in the nucleus with a certainty of 0.880. Using the SignalP analysis, it is predicted that the protein of the invention does not have a signal peptide.

SNPs and cSNPs:

Single nucleotide polymorphism analysis is detailed in Example 2. As is shown in Table 5D, in the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 5D

Cons.Pos.: 40 Depth: 5 Change: C > T
Cons.Pos.: 41 Depth: 5 Change: A > C
Cons.Pos.: 112 Depth: 17 Change: C > G
Cons.Pos.: 274 Depth: 60 Change: A > G
Cons.Pos.: 526 Depth: 45 Change: C > T
Cons.Pos.: 828 Depth: 11 Change: C > G
Cons.Pos.:1043 Depth: 12 Change: A > C
Cons.Pos.:1052 Depth: 13 Change: A > G
Cons.Pos.:1058 Depth: 14 Change: G > A
Cons.Pos.:1064 Depth: 14 Change: A > C
Cons.Pos.:1065 Depth: 14 Change: G > A
Cons.Pos.:1076 Depth: 14 Change: T > A
Cons.Pos.:1082 Depth: 13 Change: C > T
Cons.Pos.:1085 Depth: 12 Change: A > C
Cons.Pos.:1086 Depth: 12 Change: G > T
Cons.Pos.:1480 Depth: 45 Change: G > A
Cons.Pos.:1547 Depth: 55 Change: T > C
Cons.Pos.:1873 Depth: 62 Change: C > T
Cons.Pos.:1923 Depth: 61 Change: T > G
Cons.Pos.:2112 Depth: 42 Change: G > A
Cons.Pos.:2872 Depth: 9 Change: T > C

The proteins of the MLCK family have been shown to be useful in potential therapeutic applications implicated in various pathologies/disorders such as, for example, musclular dystrophy, Lesch-Nyhan syndrome and Myasthenia gravis. POLY1-4 are useful to identify novel MLCK-binding proteins, and in diagnostic and therapeutic applications, e.g. musclular dystrophy, Lesch-Nyhan syndrome and Myasthenia gravis.

POLY5-6

Calgizzarin-Like Proteins and Nucleic Acids

Calgizzarin belongs to the family of calcium binding proteins and are members of the S100 protein family. Proteins of the S100 protein family belong to the large group of EF-hand calcium-binding proteins. The expression of human calgizzarin was remarkably elevated in colorectal cancers compared with that in normal colorectal mucosa. Calgizzarin, or MLN70, is one of several genes expressed in breast cancer-derived metastatic axillary lymph nodes but not in normal lymph nodes or breast fibroadenomas. By in situ hybridization, the calgizzarin, or S100C, gene mapped to 1q21. S100A11 is part of the S100 gene cluster and is located near S100A10.

The psoriasin gene is expressed in breast cancer cell lines and in cancer cells of some breast carcinomas but not in any non-cancerous tissues examined, except skin. Another S100 gene, S100C, which was co-localized with the psoriasin gene to human chromosome 1q21-q22, was found to be expressed in most tissues and cell lines evaluated. These findings add support to the concept that the S100 genes clustered in human chromosome 1q21-q22 are individually controlled and that some of them may be involved in the regulation of cell transformation and/or differentiation.

In Northern blot analysis, 96 out of 98 genes were shown to be expressed at the same level in colon and lung carcinoma cell lines and control fibroblasts. Only two clones, including human synovial phospholipase A-2 and a homologue to rabbit calgizzarin, were expressed at different levels among these cell lines. The full sequence of human calgizzarin was determined and its expression was remarkably elevated in colorectal cancers compared with that in normal colorectal mucosa.

POLY5

A novel nucleic acid was identified on chromosome 12 that is comprised of 322 nucleotides (SEQ ID NO:9), which encodes a calgizzarin-like-like protein and is shown in Table 6A. An open reading frame was identified beginning with an ATG initiation codon at nucleotides 4-6 and ending with a TGA codon at nucleotides 316-318. The start and stop codons are in bold letters. Putative untranslated regions, if any, are found upstream from the initiation codon and downstream from the termination codon. The encoded protein having 104 amino acid residues (SEQ ID NO: 10) is presented using the one-letter code in Table 6B.

TABLE 6A
The nucleotide sequence of POLY5

>AC026105

| | |
|---|---|
| AACATGGCAAAAATCTCCGGCCCTACAGAGACTGCGCGGTGCATTGAGTCCCTGATAGCTGTTTTCCAG | (SEQ ID NO:9) |
| AAGTATGCTGGAAAGGATGGTTACAACTGCAATCTCTCCAAGACGGAGTTCCCAAGCTTCATGAATAAA | |
| GAGCTGGCTGCCTTTACAAAGAACCAGAAGGACCCCGGTGTCCTTGACCGCATGAAGAAACTGGCTGTC | |
| AGCAGCGATGGGCAGTTAGATTTCCCAAAATTTCTTAATCTGATTGGTGGCCTAGCTGCGGCTTGCCAT | |
| GACTCCTTCCTCAAGGCTGTCCCTTCCCAAAAGTGGAACTGAGGAC | |

TABLE 6B
Protein seouence encoded by the coding sejuence shown in TABLE 6A

| | |
|---|---|
| MAKISGPTETARCIESLIAVFQKYAGKDGYNCNLSKTEFPSFMNKELAAFTKNQKDPGVLDRMKKLAVSS | (SEQ ID NO:10) |
| DGQLDFPKFLNLIGGLAAACHDSFLKAVPSQKWN | |

In a search of sequence databases, it was found, for example, that the nucleic acid sequence (SEQ ID NO:9) has 292 of 325 bases (89%) identical to a Homo sapiens calgizzarin mRNA (GENBANK-ID: AA307968|acc:AA307968). In a search of sequence databases, it was found, for example, that the nucleic acid sequence has 82 of 87 bases (94%/o) identical to a Homo sapiens calgizzarin mRNA (GENBANKNEW-ID:AI907124|acc:AI907124). The full amino acid sequence of the protein of the invention was found to have 94 of 102 amino acid residues (92%) identical to, and 94 of 102 residues (92%) positive with, the 102 amino acid residue PUTATIVE S100-TYPE CALCIUM-BINDING protein from Homo sapiens (ptnr: SWISSNEW-ACC:O60417) (FIG. 5B). In addition, this protein contains the S-100 protein domains (IPR001751 as defined by Interpro) at amino acid positions 11 to 54. (Table 6C)

TABLE 6C
BLASTX of POLY5 against Putative S100 Type
Calcium-Binding Protein (SEQ ID NO:33)

>ptnr: SWISSNEW-ACC:060417 PUTATIVE S100-TYPE CALCIUM-BINDING PROTEIN
RG276003.3 - Homo sapiens (Human), 102 aa.
Length = 102
Plus Strand HSPs:
Score = 473 (166.5 bits), Expect = 1.9e-44, P = 1.9e-44
Identities = 94/102 (92%), Positives = 94/102 (92%), Frame = +1

| | | | | |
|---|---|---|---|---|
| Query: | 4 | MAKISGPTETARCIESLIAVFQKYAGKDGYNCNLSKTEFPSFMNKELAAFTKNQKDPGVL | 183 | (SEQ ID NO.:10) |
| | | \|\|\|\|\| \|\|\|\| \|\|\|\|\|\|\|\|\|\|\|\|\|\|\|\|\|\|\|\| \|\|\|\|\|\|\| \|\|\|\| \|\|\|\|\|\|\|\|\|\|\|\|\|\|\| | | |
| Sbjct: | 1 | MAKISSPTETERCIESLIAVFQKYAGKDGYNRNLSKTEFLSFMNTELAAFTKNQKDPGVL | 60 | (SEQ ID NO:33) |
| Query: | 184 | DRMKKLAVSSDGQLDFPKFLNLIGGLAAACHDSFLKAVPSQK | 309 | |
| | | \| \|\|\|\| \|\|\|\|\|\|\|\|\|\|\|\|\|\|\|\|\|\|\|\| \|\|\|\|\|\|\|\|\|\|\|\|\|\| | | |
| Sbjct: | 61 | DHMKKLDVSSDGQLDFPKFLNLIGGLAVACHDSFLKAVPSQK | 102 | |

Other polypeptide sequences with homology to POLY5 are indicated in Table 6D.

TABLE 6D

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAB58356 Lung cancer associated polypeptide seque. . . | +1 | 450 | 9.9e−42 | 1 |
| patp:AAB45541 Human S100A11 protein—Homo sapiens, 10. . . | +1 | 444 | 4.3e−41 | 1 |
| patp:AAB45540 Human S100A10 protein—Homo sapiens, 97. . . | +1 | 164 | 2.0e−11 | 1 |
| patp:AAY93605 Protein encoded by a gene encoding cellu. . . | +1 | 164 | 2.0e−11 | 1 |
| patp:AAY93492 Amino acid sequence of a potassium chann. . . | +1 | 159 | 6.8e−11 | 1 |

PSORT analysis demonstrates that POLY5 is most likely located in the mitochondrial matrix space with a certainty of 0.4494. SignalP analysis predicts that the protein does not have a signal peptide. The predicted molecular weight is 11404.0 daltons.

POLY6

A novel nucleic acid was identified on chromosome 11 that is comprised of 348 nucleotides (SEQ ID NO: 11), which encodes a calgizzarin-like-like protein and is shown in Table 7A. An open reading frame was identified beginning with an ATG initiation codon at nucleotides 5-7 and ending with a TGA codon at nucleotides 341-343. The start and stop codons are in bold letters. Putative untranslated regions, if any, are found upstream from the initiation codon and downstream from the termination codon. The encoded protein having 112 amino acid residues (SEQ ID NO:12) is presented using the one-letter code in Table 7B.

TABLE 7A
The nucleotide sequence of POLY6.

| | |
|---|---|
| >GMdj130L23_A | |
| TGCCATGAGCCCCTTTGGCAGTCTGGCGAAGCTCTTGGGTCCTTCTCAGATTGCATGGTGGTGCATCACGACCTG | (SEQ ID NO:11) |
| TGCTGTTTTCCAGAGAGGGTATGCTGGACGGGACCATAACAGCTGCAAACTCTCCCAGAGGGGGTTCCTAAACTT | |
| CATGAACACTGTACTGGTTGCCTTCACAAAGAACCAGAAGGGCTCTGGTGCCCTTGACTGCATGATGAAGAAACT | |
| GGACTTCAACTGTGATGGGCAGCTAGATTTTCAGGACTTTCTCAGTCTTACTGATGGTGTAGCTGTGGCTTGCCC | |
| TGACTCCTTCATCCCGGCTGGCCATGCCCATGAGAGAATCTGAGGTGC | |

TABLE 7B
Protein sequence encoded by the coding sequence shown in TABLE 7A

| | |
|---|---|
| MSPFGSLAKLLGPSQIAWWCITTCAVFQRGYAGRDHNSCKLSQRGFLNFMNTVLVAFTKNQKGSGALDCM | (SEQ ID NO:12) |
| MKKLDFNCDGQLDFQDFLSLTDGVAVACPDSFIPAGHAHERI | |

In a search of sequence databases, it was found, for example, that the nucleic acid sequence (SEQ ID NO:11) has 249 of 322 bases (77%) identical to a Homo sapiens calgizzarin mRNA (GENBANK-ID: HUMCOLO|acc:D38583). The amino acid sequence of the protein of the invention was found to have 55 of 84 amino acid residues (65%) identical to, and 67 of 84 residues (79%) positive with, the 98 amino acid residue calgizzarin protein from Mus musculus (ptnr: SWISSPROT-ACC:P50543) (FIG. 7B). The global sequence homology (as defined by GAP global sequence alignment with the fill length sequence of this protein) is 60% amino acid similarity and 55% amino acid identity. In addition, this protein contains the following protein domains (as defined by Interpro) at the indicated amino acid positions: S-100 (IPR001751) at amino acid positions 20 to 60; and EF HAND (IPR002048) at amino acid positions 66 to 94. (Table 7C).

TABLE 7C
BLASTX of POLY6 AGAINST CALGIZZARIN (ENDOTHELIAL
MONOCYTE-ACTIVATING POLYPEPTIDE) (EMAP)—Mus musculus (Mouse) (SEQ ID NO:34)

>ptnr:SWISSPROT-ACC:P50543 CALGIZZARIN (ENDOTHELIAL MONOCYTE-ACTIVATING
POLYPEPTIDE) (EMAP)—Mus musculus (Mouse), 98 aa.
Length = 98
Plus Strand HSPs:
Score = 273 (96.1 bits), Expect = 2.9e−23, P = 2.9e−23
Identities = 55/84 (65%), Positives = 67/84 (79%), Frame = +2

| | | | | |
|---|---|---|---|---|
| Query: | 62 | CITTC-AVFQRGYAGRDHNSCKLSQRGFLNFMNTVLVAFTKNQKGSGALDCMMKKLDFNC | 238 | |
| | | \|\| + \|\|\|\|+ \|+\|+\| \|+ +\|\|+ \|\|+\|\|\|\| \| \|\|\|\|\|\|\| \| \|\| \|\|\|\|\|\| \|\| | | |
| Sbjct: | 8 | CIESLIAVFQK-YSGKDGNNTQLSKTEFLSFMNTELAAFTKNQKDPGVLDRMMKKLDLNC | 66 | |
| Query: | 239 | DGQLDFQDFLSLTDGVAVACPDSFI | 313 | (SEQ ID NO.12) |
| | | \|\|\|\|\|\|\|+\|\|+\| \|+\|+\|\| \|\|\|\| | | |
| Sbjct: | 67 | DGQLDFQEFLNLIGGLAIACHDSFI | 91 | (SEQ ID NO:34) |

POLY6 homology with other polypeptide sequences is shown in Table 7D.

TABLE 7D.

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAB58356 Lung cancer associated polypeptide seque. . . | +1 | 450 | 9.9e−42 | 1 |
| patp:AAB45541 Human S100A11 protein—Homo sapiens, 10. . . | +1 | 444 | 4.3e−41 | 1 |
| patp:AAB45540 Human S100A10 protein—Homo sapiens, 97. . . | +1 | 164 | 2.0e−11 | 1 |
| patp:AAY93605 Protein encoded by a gene encoding cellu. . . | +1 | 164 | 2.0e−11 | 1 |
| patp:AAY93492 Amino acid sequence of a potassium chann. . . | +1 | 159 | 6.8e−11 | 1 |

PSORT analysis demonstrates that POLY6 is most likely located in the endoplasmic reticulum with a certainty of 0.55. SignalP analysis is suggests that POLY6 has a signal peptide with most likely cleavage site between pos. 32 and 33: GYA-GR in SEQ ID NO:12. The predicted molecular weight is 12281.0 daltons.

The above defined information for this invention suggests that this calgizzarin-like protein may function as a member of a “calgizzarin family”. The expression of human calgizzarin has been found to be remarkably elevated in colorectal cancers compared with that in normal colorectal cancers. Calgizzarin has also been shown to be one of several genes expressed in breast cancer-derived metastatic axillary lymph nodes but not in normal lymph nodes or breast fibraodenomas. Therefore, the novel nucleic acids and proteins identified here may be useful in potential therapeutic applications implicated in (but not limited to) various pathologies and disorders such as lung, colorectal cancers and leukemia, neuropsychiatric disorders including schizophrenia, medullary cystic kidney disease, and anemia, and/or other pathologies and disorders.

POLY 7-POLY8

Beta-Thymosin-Like Proteins, Polypeptides and Nucleic Acids

The beta-thymosins are a family of related peptides, initially isolated from calf thymus but known to be present in a wide variety of mammalian and other vertebrate cells and tissues. Thymosin-beta-4 was the first member of the family to be characterized. Although TMSB4 was initially proposed to be a thymic hormone acting at early stages of T-cell maturation, the high concentration of the protein and its mRNA in a number of other tissues and cells, as well as the lack of an identifiable secretory signal sequence, suggested that it had a general function in many cell types. This was confirmed by the demonstration that TMSB4 forms a 1:1 complex with G-actin in blood platelets and other evidence that it is the only known G-actin-sequestering protein present at high enough levels in blood platelets to account for the high levels of G-actin in those cells. Thymosin-beta-4 induces the expression of terminal deoxynucleotidyl transferase activity in vivo and in vitro, inhibits the migration of macrophages, and stimulates the secretion of hypothalamic luteinizing hormone-releasing hormone. The protein was originally isolated from a partially purified extract of calf thymus, thymosin fraction 5,which induced differentiation of T cells and was partially effective in some immunocompromised animals.

Further studies demonstrated that the molecule is ubiquitous; it had been found in all tissues and cell lines analyzed. It is found in highest concentrations in spleen, thymus, lung, and peritoneal macrophages. Thymosin-beta-4 is an actin monomer sequestering protein that may have a critical role in modulating the dynamics of actin polymerization and depolymerization in nonmuscle cells. Its regulatory role is consistent with the many examples of transcriptional regulation of T-beta-4 and of tissue-specific expression. Lymphocytes have a unique T-beta-4 transcript relative to the ubiquitous transcript found in many other tissues and cells. Rat thymosin-beta-4 is synthesized as a 44-amino acid propeptide which is processed into a 43-amino acid peptide by removal of the first methionyl residue. The molecule does not have a signal peptide. Human thymosin-beta-4 has a high degree of homology to rat thymosin-beta-4; the coding regions differ by only 9 nucleotides, and these are all silent base changes.

Prostate carcinoma is the most prevalent form of cancer in males and the second leading cause of cancer death among older males. The use of the serum prostate-specific antigen (PSA) test permits early detection of human prostate cancer; however, early detection has not been accompanied by an improvement in determining which tumors may progress to the metastatic stage. The process of tumor metastasis is a multistage event involving local invasion and destruction of extracellular matrix; intravasation into blood vessels, lymphatics or other channels of transport; survival in the circulation; extravasation out of the vessels into the secondary site; and growth in the new location. Common to many components of the metastatic process is the requirement for tumor cell motility. A well-characterized series of cell lines that showed varying metastatic potential was developed from the Dunning rat prostate carcinoma. There is a direct correlation between cell motility and metastatic potential in the Dunning cell lines. In studies comparing gene expression in poorly and highly motile metastatic cell lines derived from Dunning rat prostate carcinoma using differential mRNA display, a novel member of the thymosin-beta family of actin-binding molecules was found (see OMIM-300159). The molecule, named thymosin-beta-15 by them, was found to deregulate motility in prostate cells directly. In addition, it was expressed in advanced human prostate cancer specimens, but not in normal human prostate or benign prostatic hyperplasia, suggesting its potential use as a new marker for prostate carcinoma progression. Thymosin-beta-15 levels correlated positively with the Gleason tumor grade. Upregulation of thymosin-beta-15 as a positive motility factor and the down regulation of the motility suppressor KAI1 (OMIM-600623) provide the ‘yin and yang’ for metastasis; he speculated that these pathways may provide a new target for therapy.

The below-described information for POLY7-8 suggests that the POLY7-8 beta thymosin-like polypeptides may function as members of a “beta thymosin family”. Therefore, the novel nucleic acids and proteins identified here may be useful in potential therapeutic applications implicated in (but not limited to) various pathologies and disorders as a protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The POLY7-8 nucleic acids and proteins of the invention are useful in potential therapeutic applications implicated in cancer including but not limited to prostate cancer, immunological and autoimmune disorders (ie hyperthyroidism), angiogenesis and wound healing, modulation of apoptosis, neurodegenerative and neuropsychiatric disorders, age-related disorders, and other pathological disorders involving spleen, thymus, lung, and peritoneal macrophages and/or other pathologies and disorders. For example, a cDNA encoding the beta thymosin-like polypeptide may be useful in gene therapy, and the beta thymosin-like polypeptide may be useful when administered to a subject in need thereof. By way of nonlimiting example, the compositions of the present invention will have efficacy for treatment of patients suffering from cancer including but not limited to prostate cancer, immunological and autoimmune disorders (ie hyperthyroidism), angiogenesis and wound healing, modulation of apoptosis, neurodegenerative and neuropsychiatric disorders, age-related disorders, and other pathological disorders involving spleen, thymus, lung, and peritoneal macrophages. The novel nucleic acid encoding beta thymosin-like polypeptide, and the beta thymosin-like polypeptide of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

POLY7

A POLY7 nucleic acid that is comprised of 170 nucleotides (SEQ ID NO:13) was identified on chromosome 11, which encodes a beta thymosin-like protein and is shown in Table 8A. An open reading frame was identified beginning with nucleotide 1 and ending with a TAA codon at nucleotides 156-158. The start and stop codons are in bold letters. A putative untranslated region was found downstream from the termination codon. The encoded protein having 51 amino acid residues (SEQ ID NO: 14) is presented using the one-letter code in Table 8B.

TABLE 8A
The nucleotide sequence of POLY7.

| | |
|---|---|
| >AP001591_A | |
| AGGCTGGTCTGGAACTCCTGGCCTCAAGTGATCCACCTGACTTTGCCTCCCTCCCGAAGAGAGATAAGTC | (SEQ ID NO:13) |
| GAAACTGAAGAAGACAGAAGTGCAAGAGAAAAATCCACTGCCTTCCAAAGAAATGATTGAACAGGAGAAG | |
| CAAGCTGGTGAATCGTAATGAGGCATGTGC | |

TABLE 8B
Protein sequence encoded by the coding sequence shown in TABLE 8A

| | |
|---|---|
| AGLELLASSDPPDFASLPKRDKSKLKKTEVQEKNPLPSKEMIEQEKQAGES | (SEQ ID NO:14) |

In a search of sequence databases, it was found, for example, that the nucleic acid sequence (SEQ ID NO:13) has 118 of 142 bases (83%) identical to a human beta thymosin mRNA (GENBANK-ID: HUMTHYB4|ac : M17733). The amino acid sequence of the protein of the invention was found to have 35 of 43 amino acid residues (81%) identical to, and 36 of 43 residues (83%) positive with, the 43 amino acid residue thymosin beta 4 protein from Homo sapiens (ptnr: SWISSPROT-ACC:P01253) (Table 8C). The global sequence homology is 84% amino acid similarity and 81% amino acid identity. In addition, this protein contains the thymosin protein domain (as defined by Interpro# IPR001152) at amino acid positions 9 to 49.

TABLE 8C

BLASTX identity search against HEMATOPOIETIC SYSTEM REGULATORY
PEPTIDE—Homo sapiens (Human) (SEQ ID NO: 35)

>ptnr:SWISSPROT-ACC:P01253 THYMOSIN BETA-4 (FX) [CONTAINS: HEMATOPOTETIC
SYSTEM REGULATORY PEPTIDE]—Homo sapiens (Human), Bos taurus (Bovine),,
43 aa.
Length = 43
Plus Strand HSPs:
Score = 173 (60.9 bits), Expect = 1.3e−12, P = 1.3e−12
Identities = 35/43 (81%), Positives = 36/43 (83%), Frame = +3

| | | | | |
|---|---|---|---|---|
| Query: | 27 | SDPPDFASLPKRDKSKLKKTEVQEKNPLPSKEMIEQEKQAGES | 155 | (SEQ ID NO.14) |
| | | \|\| \|\| \| + \| \|\|\|\|\|\|\|\|\| \|\|\|\|\|\|\|\|\|\| \|\|\|\|\|\|\|\|\|\| | | |
| Sbjct: | 1 | SDKPDMAEIEKFDKSKLKKTETQEKNPLPSKETIEQEKQAGES | 43 | (SEQ ID NO:35) |

Other polypeptide sequence with homology to POLY7 are indicated in Table 8D.

TABLE 8D

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAY76578 Human ovarian tumor EST fragment encoded. . . | −2 | 181 | 3.2e−13 | 1 |
| patp:AAP81169 Protein produced in myeloma cell differe. . . | +3 | 173 | 2.2e−12 | 1 |
| patp:AAR04593 Thbeta4-tumour necrosis factor fusion pe. . . | +3 | 173 | 2.2e−12 | 1 |
| patpAAR96921 Thymosin beta 4—Synthetic, 43 aa. | +3 | 173 | 2.2e−12 | 1 |
| patp:AAW81507 Thymosin beta 4, X isoform (TB4X) gene p. . . | +3 | 173 | 2.2e−12 | 1 |

PSORT analysis demonstrates that POLY7 is most likely located in the cytoplasm (certainty=0.45). SIGNALP analysis suggests that POLY7 does not appear to contain a predicted signal peptide. The predicted molecular weight is 5624.3 daltons.

Quantitative expression of POLY7 was assessed as described in Example 4.

POLY8

A novel nucleic acid was identified on chromosome 6 that is comprised of 227 nucleotides (SEQ ID NO: 15), which encodes a beta thymosin-like protein is shown in Table 9A. An open reading frame was identified beginning with an ATG initiation codon at nucleotides 4-6 and ending with a ATG codon at nucleotides 217-219. The start and stop codons are in bold letters. A putative untranslated region was found upstream from the initiation codon and downstream from the termination codon. The encoded protein having 71 amino acid residues (SEQ ID NO:16) is presented using the one-letter code in Table 9B.

TABLE 9A
The nucleotide sequence of POLY8.

| | |
|---|---|
| >AC025535_B | |
| AGTATGGTCTCAGCCCAGCGTTTCACGAGTCTTCAAGCCTTCAGGCTTTCTTTAATCAAGATGAGTGATA | (SEQ ID NO:15) |
| AACCCAACTTGTCAGAAGTGAAGTTTGACAGGTCAAAATTGAAGAAAACTAACACTGGAGAAAAAAATAG | |
| GCTTTCTTCCAAGGAAACTATCCAGCAGGAGAAAGAGGCAGGAGAATCGCTTGAACCCGGGAGGCTCAGG | |
| TTGTGGTGAGCCGATAT | |

TABLE 9B
PROTEIN SEQUENCE ENCODED BY THE CODING SEQUENCE SHOWN IN TABLE 8A

| | |
|---|---|
| MVSAQRFTSLQAFRLSLIKMSDKPNLSEVKFDRSKLKKTNTGEKNRLSSKETIQQEKEAGESLEPGRLRLW | (SEQ ID NO:16) |

In a search of sequence databases, it was found, for example, that the nucleic acid sequence (SEQ ID NO:15) has 145 of 166 bases (87%) identical to a human beta thymosin mRNA (GENBANK-ID: D82345|acc:D82345). The full amino acid sequence of the protein of the invention was found to have 35 of 40 amino acid residues (87%) identical to, and 36 of 40 residues (90%) positive with, the 45 amino acid residue thymosin beta protein from Homo sapiens (ptnr: PIR-ID:JC5274) (Table 9C). The global sequence homology is 81% amino acid similarity and 80% amino acid identity. In addition, this protein contains the thymosin protein domain (as defined by Interpro# IPR0011152) at amino acid positions 21 to 60.

TABLE 9C
BLASTX IDENTITY SEARCH AGAINST THYMOSIN BETA - HUMAN (SEO ID NO:36)

>ptnr:PIR-ID:JC5274 thymosin beta - human
Length = 45
Plus Strand HSPs:
Score = 161 (56.7 bits), Expect = 2.4e−11, P = 2.4e−11
Identities = 35/40 (87%), Positives = 36/40 (90%), Frame = +1

| | | | | |
|---|---|---|---|---|
| Query: | 61 | MSDKPNLSEV-KFDRSKLKKTNTGEKNRLSSKETIQQEKE | 177 | |
| | | \|\|\|\|\|+\|\|\|\| \|\|\|\|\|\|\|\|\|\|\|\| \|\|\| \| \|\|\|\|\|\|\|\|\|\| | | |
| Sbjct: | 1 | MSDKPDLSEVEKFDRSKLKKTNTEEKNTLPSKETIQQEKE | 40 | (SEQ ID NO:36) |

Other polypeptide sequences with homology to POLY8 are described in Table 9D.

TABLE 9D

| | | |
|---|---|---|
| | | Smallest |
| | | Sum |
| | | Probabili- |
| Reading | High | ty |

| | | | | |
|---|---|---|---|---|
| Sequences producing High-scoring Segment Pairs: | Frame | Score | P(N) | N |

| | | | | |
|---|---|---|---|---|
| patp:AAP81169 Protein produced in myeloma cell differe. . . | +1 | 167 | 9.7e−12 | 1 |
| patp:AAW14281 Human neuroblastoma-specific thymosin-be. . . | +1 | 161 | 4.2e−11 | 1 |
| patp:AAW81508 Thymosin beta 4, Y isoform (TB4Y) gene p. . . | +1 | 159 | 6.8e−11 | 1 |
| patp:AAW81507 Thymosin beta 4, X isoform (TB4X) gene p. . . | +1 | 158 | 8.7e−11 | 1 |
| patp:AAB53712 Human colon cancer antigen protein seque. . . | +1 | 158 | 8.7e−11 | 1 |
| patp:AAY91956 Human cytoskeleton associated protein 11. . . | +1 | 158 | 8.7e−11 | 1 |
| patp:AAW46486 Human thymosin beta-15 protein - Homo sa. . . | +1 | 156 | 1.4e−10 | 1 |
| patp:AAW36056 Human thymosin beta-15 protein sequence. . . | +1 | 154 | 2.3e−10 | 1 |
| patp:AAW68573 Rat thymosin-beta15 protein - Rattus sp,. . . | +1 | 154 | 2.3e−10 | 1 |
| patp:AAW44275 Human thymosin beta 15 - Homo sapiens, 4. . . | +1 | 154 | 2.3e−10 | 1 |

PSORT analysis demonstrates that POLY8 is most likely located outside in the mitochondrial intermembrane space (certainty=0.88). SignalP analysis suggests that POLY8 does not appear to contain a predictable signal peptide. The predicted molecular weight is 8197.3 daltons.

Quantitative expression of POLY8 was assessed as described in Example 4.

Thymosin-beta-4, a member of the beta thymosin family has been shown to be a potent wound healing factor. Beta thymosin proteins have also been found to be useful in potential therapeutic applications implicated in cancers, immunological and autoimmune disorders, angiogenesis, modulation of apoptosis, neurodegenerative and neuropsychiatric disorders, age-related disorders and other pathological disorders. Therefore, POLY7-8 are useful to identify novel beta thymosin-binding protein family members.

POLY9: Novel Protein Resembling Ras Suppressor Protein and Nucleic Acids

Experimental evidence from human cancer, animal tumor models and in vitro tissue culture assays of transformation indicates that activation of the signal transduction pathway regulated by the Ras GTPases can play a critical role in the development of neoplasia. Activating mutations of the ras proto-oncogene are common genetic alterations in specific human tumors. In addition to mutational activation of a ras gene itself, the equivalent of an activated Ras signal may result from a loss of function of the genes negatively regulating Ras p21 signaling.

A study of the Ras suppressor molecules as tumor suppressors requires the identification of human tumors in which these genes are mutated or silenced. Glioblastoma is a tumor in which RSU-1 is altered. Mouse Rsu-1 (formerly referred to as Rsp-1) is a novel cDNA capable of suppressing Ki-ras transformation. Rsu-1 is phylogenetically conserved and ubiquitously expressed, suggesting that it may interact with other highly conserved proteins and function in a Ras signal transduction pathway in higher eukaryotes. Human RSU1 cDNA was isolated from a lambda gt10 human primary skin fibroblast cDNA library and showed that the human protein exhibits more than 95% conservation with the murine Rsu-1 at the amino acid level. By hybridization of a human RSU1 cDNA probe to a set of hamster-human somatic cell hybrids, RSU1 gene maps to chromosome 10. Several neoplastic disease loci have been mapped to chromosome 10.

Rsu-1, which was isolated based on its ability to suppress transformation by v-Ras, is a highly conserved gene which shares homology with yeast adenylyl cyclase in the region required for activation by Ras. Genomic DNA clones of human RSU-1 have been isolated and used as a probe for fluorescence in situ hybridization (FISH) to assign RSU-1 to 10p13, confirming the previous results of somatic cell hybrid mapping localizing RSU-1 to chromosome 10. Screening of more than 20 human tumor cell lines for RSU-1 expression revealed that most cell lines contained abundant RSU-1 RNA and protein.

The above defined information for this invention suggests that this protein resembling Ras suppressor protein may function as a member of a “Ras Suppressor Protein family”. Therefore, the novel nucleic acids and proteins identified herein may be useful in potential therapeutic applications include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in various cancers including but not limited to leukemia, melanomas, carcinomas, sarcomas, bladder, mammary, renal-pelvic, ovarian, lung and colon cancer, and human solid tumors and urinary tract tumors; and other types of neoplastic disorders and/or other pathologies and disorders. For example, a cDNA encoding the protein resembling Ras suppressor protein may be useful in gene therapy, and the protein resembling Ras suppressor protein may be useful when administered to a subject in need thereof. By way of nonlimiting example, the compositions of the present invention will have efficacy for treatment of patients suffering from various cancers including but not limited to leukemia, melanomas, carcinomas, sarcomas, bladder, mammary, renal-pelvic, ovarian, lung and colon cancer, and human solid tumors and urinary tract tumors; and other types of neoplastic disorders. The novel nucleic acid encoding protein resembling Ras suppressor protein, and the protein resembling Ras suppressor protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

A POLY9 nucleic acid that is comprised of 826 nucleotides (SEQ ID NO: 17) was identified on chromosome 10. This POLY9 nucleic acid encodes a ras suppressor-like protein and is shown in Table 10A. An open reading frame was identified beginning with an ATG initiation codon at nucleotides 9-11 and ending with a ATG codon at nucleotides 819-821. The start and stop codons are in bold letters. A putative untranslated region was found upstream from the initiation codon and downstream from the termination codon. The encoded protein having 71 amino acid residues (SEQ ID NO: 18) is presented using the one-letter code in Table 10B.

TABLE 10A
THE NUCLEOTIDE SEQUENCE OF POLY9.

| | |
|---|---|
| >GM87333647_A | |
| TCACGACCATGTCCAAGACTCTGAAAAAGTTTGTGGAGAGCCAGGAAGTGGACAGGGTGACTAGGTCATC | (SEQ ID NO:17) |
| TACAAACATGCTGTATGTCAATGGCACATTTTCCTTATCCCATACCATACAACTGGTCCTCAGCCATAAC | |
| AAGCTCACAGTGGTGCCACCAAACACAGCAGAACTGAAGAATTTGGAAGTGCTCAACTTCCTTAATAGCC | |
| AGATTGAGGAGCTGCCCACACAGATCGGCAGCCTTCAGAAACTCAAACACATGAACCTGGGCATGAATGG | |
| GCTAAATACTTTGCCTGAAGGATTTTGCTTTCTACCAGCTCTTGACCTTCTGGACTTGATGTACAATTTG | |
| AATGAGAATTCTCTTCCTGGAAACTTCATCTACCTTACTACCTTCCGTGCACTCTATGTAAGTGACAATG | |
| ATTTTAAAATCCTGCAACCAGATATTAGGAAGCTCACAAAGTTGCAGATACCCAGCTTTAGGGATAACAA | |
| CCTGATCTTGCAGCCTAGGGAAACTGGGGAGTTTACCCAGCTTAAGGAACTCAACATTCAGGGCAACTGC | |
| CTGACCCTTCTGCTCCCAGAACTAGGAAACTTATATTTAACTGGTCAGAAGAAGGTATGCAAAGTGGAGA | |
| ACAGCCCCTGGGTTACCCCAATTGCTGGCCAGTTCCAGCTTGATGTGTCCTGTGTGTCTGAATGTGTCTG | |
| TTCTGAGACATATGAGTACCTCTATGGGCAGCACATGCAGGCAAATCCAGAACCACCAAAACATAATAAT | |
| CACAAATCAGAAAAGATGAGCTGGAAACACCTGACAGACAGTAACAAATAAGAGGT | |

TABLE 10B
PROTEIN SEQUENCE ENCODED BY THE CODING SEQUENCE SHOWN IN TABLE 10A

| | |
|---|---|
| MSKTLKKFVESQEVDRVTRSSTNMLYVNGTFSLSHTIQLVLSHNKLTVVPPNTAELKNLEVLNFLNSQIEELPTQI | (SEQ ID NO:18) |
| GSLQKLKHMNLGMNGLNTLPEGFCFLPALDLLDLMYNLNENSLPGNFIYLTTFRALYVSDNDFKILQPDIRKLTKL | |
| QIPSFRDNNLILQPRETGEFTQLKELNIQGNCLTLLLPELGNLYLTGQKKVCKVENSPWVTPIAGQFQLDVSCVSE | |
| CVCSETYEYLYGQHMQANPEPPKHNNHKSEKMSWKHLTDSNK | |

In a search of sequence databases, it was found, for example, that the nucleic acid sequence (SEQ ID NO:17) has 659 of 786 bases (83%) identical to a Homo sapiens Ras Suppressor Protein mRNA (GENBANK-ID: HUMRSU1A|acc:L12535). The full amino acid sequence of the protein of the invention was found to have 199 of 277 amino acid residues (71%) identical to, and 222 of 277 residues (80%) positive with, the 277 amino acid residue Ras suppressor protein 1 protein from Homo sapiens (ptnr: SWISSPROT-ACC:Q15404) (Table 10° C.). The global sequence homology (as defined by GAP global sequence alignment with the full length sequence of this protein) is 76% amino acid similarity and 74% amino acid identity. In addition, this protein contains the following protein domains (as defined by Interpro, an integrated resource of protein domains and functional sites (http://www.ebi.ac.uk/interpro/index.html)) at the indicated amino acid positions: five leucine-rich repeat domains (IPR001611) at amino acid positions 35 to 57, 58 to 80, 81 to 103, 128 to 150 and 174 to 196; and the serine protease inhibitor Squash domain (IPR000737) at amino acid positions 222 to 239.

TABLE 10C

BLASTX IDENTITY SEARCH FOR THE PROTEIN RESEMBLING RAS SUPPRESSOR PROTEIN
OF THE INVENTION (SEQ ID NO:37)

>ptnr:SWISSPROT-ACC:Q15404 RAS SUPPRESSOR PROTEIN 1 (RSU-1) (RSP-1 PRO-
TEIN) (RSP-1) - Homo sapiens (Human), 277 aa.
Length = 277
Plus Strand HSPs:
Score = 962 (338.6 bits), Expect = 3.2e−96, P = 3.2e−96
Identities = 199/277 (71%), Positives = 222/277 (80%), Frame = +3

| | | | | |
|---|---|---|---|---|
| Query: | 9 | MSKTLKKFVESQ------EVDRVTRSSTNMLYVNGTFSLSHTIQLVLSHNKLTVVPPNTA | 170 | |
| | | \|\|\|+\|\|\| \|\| \|\|\| \| +\|\|\| \|\|\| \|+\|\|\| \|\|\|\|\|\|\|\|\|\|+\|\|\|\| \| | | |
| Sbjct: | 1 | MSKSLKKLVEESREKNQPEVDMSDRGISNMLDVNGLFTLSHITQLVLSHNKLTMVPPNIA | 60 | |
| Query: | 71 | ELKNLEVLNFLNSQIEELPTQIGSLQKLKHMNLGMNGLNTLPEGFCFLPALDLLDLMYN- | 347 | |
| | | \|\|\|\|\|\|\|\|\|\| \|+\|\|\|\|\|\|\|\|\| \|\|\|\|\|\|\|+\|\|\|\|\| \|\|\|\|\| \|\| \|\|\|\|++\|\|\| \|\| | | |
| Sbjct: | 61 | ELKNLEVLNFFNNQIEELPTQISSLQKLKHLNLGMNRLNTLPRGFGSLPALEVLDLTYNN | 120 | |
| Query: | 348 | LNENSLPGNFIYLTTFRALYVSDNDFKILQPDIRKLTKLQIPSFRDNNLILQPRETGEFT | 527 | |
| | | \|+\|\|\|\|\|\|\|\| \|\|\|\| \|\|\|\|+\|\|\|\|\|+\|\| \|\|\| \|\|\|\|\|\|\| \| \|\|\|+\|\| \|+\| \|\| \| | | |
| Sbjct: | 121 | LSENSLPGNFFYLTTLRALYLSDNDFEILPPDIGKLTKLQILSLRDNDLISLPKEIGELT | 180 | |
| Query: | 528 | QLKELNIQGNCLTLLLPELGNLYLTGQKKVCKVENSPWVTPIAGQFQLDVSCVSECVCSE | 707 | |
| | | \|\|\|\|\|+\|\|\|\| \|\|+\| \|\|\|\|\|\| \|\|\|\|\|+\| \| \|\|+\|\|\|\|\|\|\| \|\|\|\| \|\| \| \| + \|\| | | |
| Sbjct: | 181 | QLKELHIQGNRLTVLPPELGNLDLTGQKQVFKAENNPWVTPIADQFQLGVSHVFEYIRSE | 240 | |
| Query: | 708 | TYEYLYGQHMQANPEPPKHNNHKSEKMSWKHLTDSNK | 818 | (SEQ ID NO:18) |
| | | \|\|+\|\|\|\|+\|\|\|\|\|\|\|\|\|\| \|\| \|\|+\|+\| \| \| \|+ | | |
| Sbjct: | 241 | TYKYLYGRHMQANPEPPKKNNDKSKKISRKPLAAKNR | 277 | (SEQ ID NO:37) |

Other polypeptide sequences with homology to POLY9 are identified in TABLE 10D.

TABLE 10D

| | | | Smallest | |
|---|---|---|---|---|
| | | | Sum | |
| | | | Probabili- | |
| | Reading | High | ty | |
| Sequences producing High-scoring Segment Pairs: | Frame | Score | P(N) | N |

| | | | | |
|---|---|---|---|---|
| patp:AAG00223 Human secreted protein, SEQ ID NO: 4304. . . | +3 | 430 | 1.3e−39 | 1 |
| patp:AAG35764 Arabidopsis thaliana protein fragment SE. . . | +3 | 197 | 3.2e−13 | 1 |
| patp:AAG35763 Arabidopsis thaliana protein fragment SE. . . | +3 | 197 | 3.2e−13 | 1 |
| patp:AAG35762 Arabidopsis thaliana protein fragment SE. . . | +3 | 197 | 3.2e−13 | 1 |
| patp:AAB60749 Gene 2 related peptide #1 - Homo sapiens. . . | +3 | 190 | 3.7e−12 | 1 |
| patp:AAB60750 Gene 2 related peptide #2 - Homo sapiens. . . | +3 | 190 | 3.7e−12 | 1 |
| patp:AAB60703 Human secreted protein #2 - Homo sapiens. . . | +3 | 190 | 4.3e−12 | 1 |
| patp:AAY13376 Amino acid sequence of protein PRO239 -. . . | +3 | 174 | 5.3e−10 | 1 |

PSORT analysis demonstrates that POLY9 is most likely localized in the cytoplasm (certainty=0.45). SignalP analysis suggests that POLY9 does not appear to contain a signal peptide. The predicted molecular weight is 30762.1 daltons.

Quantitative expression of POLY9 was assessed as described in Example 4.

The ras suppressor protein has been shown to be useful in potential therapeutic applications implicated in various cancers including but not limited to leukemia, melanomas, carcinomas, sarcomas, bladder, mammary, renal-pelvic, ovarian, lung and colon cancer, and human solid tumors and urinary tract tumors; and other types of neoplastic disorders and/or other pathologies and disorders.

POLY10: Novel Cerebellin-Like Protein and Nucleic Acids

Precerebellin is a large protein with distant homology to the noncollagen domain of complement component C1qB. Its mRNA is highly enriched in the cerebellum. Precerebellin gives rise to several truncated derivatives, including the hexadecapeptide cerebellin which is highly enriched in postsynaptic structures of cerebellar Purkinje cells in cartwheel neurons of the dorsal cochlear nucleus. The “staggerer” mutation appears to lack cerebellin completely. The murine homolog of precerebellin, (Cbln1), and a closely related gene, Cbln2 (600433) were cloned and the predicted amino acid sequence of which is 88% identical to the carboxy-terminal region of Cbln1. Cbln1 was mapped to the central region of chromosome 8, 2.3 cM distal of Junb and 6.0 cM proximal of Mt1. JUNB maps to human 19p13.2 and MT1 maps to human 16q13. Cbln2 maps to the distal end of mouse chromosome 18, 1.7 cM telomeric of Mbp, predicting an 18q23 location for the human homolog.

The expression of cerebellin and cerebellin mRNA was studied by radioimmunoassay and Northern blot analysis in the human brain, adrenal gland and the tumour tissues of adrenal tumour, ganglioneuroblastoma and neuroblastoma. Immunoreactive cerebellin was detected in every region of brain studied, with the highest concentrations found in the hemisphere of the cerebellum and the vermis of the cerebellum. Immunoreactive cerebellin was also detected in the pituitary, the spinal cord and the normal parts of adrenal glands and some tumour tissues, such as phaeochromocytomas, cortisol-producing adrenocortical adenomas, ganglioneuroblastomas and neuroblastomas. Northern blot analysis showed that cerebellin mRNA was highly expressed in the hemisphere and vermis of the cerebellum. Cerebellin mRNA was also expressed in other regions of the brain and the tumour tissues of phaeochromocytoma, cortisol-producing adrenocortical adenoma, ganglioneuroblastoma and neuroblastoma. Immunocytochemistry of the normal adrenal gland showed that immunoreactive cerebellin was localized in the adrenal medulla. The present study has shown the expression of cerebellin and cerebellin mRNA, not only in the cerebellum but also in other regions of the brain and some tumours, such as cortisol-producing adrenocortical adenoma, phaeochromocytoma and neuroblastoma. These findings suggest possible pathophysiological roles of cerebellin peptides, not only in the cerebellum, but also in the extra-cerebellar tissues.

Four neuropeptides; cerebellin, corticotropin-releasing hormone (CRH), neuropeptide Y and somatostatin were studied by radioimmunoassay in the postmortem human brains obtained from three patients with olivopontocerebellar atrophy (OPCA) and one with Shy-Drager syndrome. Significant decreases in cerebellin and CRH concentrations were found in the cerebellar hemisphere of these diseases compared with controls. These findings suggest important pathophysiological roles of cerebellin and CRH in these cerebellar diseases. Such significant decreases were not found in neuropeptide Y and somatostatin.

The below-described information for POLY10 suggests that the POLY10 cerebellin-like protein may function as a member of a “cerebellin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The POLY12 nucleic acids and proteins of the invention are useful in potential therapeutic applications implicated in olivopontocerebellar atrophy (OPCA), Shy-Drager syndrome, ‘staggerer syndrome’, various cancers including but not limited to brain and adrenal gland tumours, including phaeochromocytomas, cortisol-producing adrenocortical adenomas, ganglioneuroblastomas and neuroblastomas and/or other pathologies and disorders. For example, a cDNA encoding the cerebellin-like protein may be useful in gene therapy, and the cerebellin-like protein may be useful when administered to a subject in need thereof. By way of nonlimiting example, the POLY12 compositions of the present invention will have efficacy for treatment of patients suffering from olivopontocerebellar atrophy (OPCA), Shy-Drager syndrome, ‘staggerer syndrome’, various cancers including but not limited to brain and adrenal gland tumours, including phaeochromocytomas, cortisol-producing adrenocortical adenomas, ganglioneuroblastomas and neuroblastomas. The novel nucleic acid encoding cerebellin-like protein, and the cerebellin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

A novel nucleic acid that is comprised of 614 nucleotides (SEQ ID NO: 19) was identified on chromosome 30. This POLY10 nucleic acid encodes a cerebellin-like protein and is shown in Table 11A. An open reading frame was identified beginning with an ATG initiation codon at nucleotides 4-6 and ending with a TAG codon at nucleotides 607-609. The start and stop codons are in bold letters. The encoded protein having 201 amino acid residues (SEQ ID NO:20) is presented using the one-letter code in Table 11B.

TABLE 11A
THE NUCLEOTIDE SEQUENCE OF POLY10.

| | |
|---|---|
| >ba458e15_A | |
| ACCATGGGCTCCGGGCGCCGGGCGCTGTCCGCGGTGCCGGCCGTGCTGCTGGTCCTCACGCTGCCGGGGC | (SEQ ID NO:19) |
| TGCCCGTCTGGGCACAGAACGACACGGAGCCCATCGTGCTGGAGGGCAAGTGTCTGGTGGTGTGCGACTC | |
| GAACCCGGCCACGGACTCCAAGGGCTCCTCTTCCTCCCCGCTGGGGATATCGGTCCGGGCGGCCAACTCC | |
| AAGGTCGCCTTCTCGGCGGTGCGGAGCACCAACCACGAGCCATCCGAGATGAGCAACAAGACGCGCATCA | |
| TTTACTTCGATCAGATCCTGGTGAATGTGGGTAATTTTTTCACATTGGAGTCTGTCTTTGTAGCACCAAG | |
| AAAAGGAATTTACAGTTTCAGTTTTCACGTGATTAAAGTCTACCAGAGCCAAACTATCCAGGTTAACTTG | |
| ATGTTAAATGGAAAACCAGTAATATCTGCCTTTGCGGGGGACAAAGATGTTACTCGTGAAGCTGCCACGA | |
| ATGGTGTCCTGCTCTACCTAGATAAAGAGGATAAGGTTTACCTAAAACTGGAGAAAGGTAATTTGGTTGG | |
| AGGCTGGCAGTATTCCACGTTTTCTGGCTTTCTGGTGTTCCCCCTATAGGATTC | |

TABLE 11B
PROTEIN SEQUENCE ENCODED BY THE CODING SEQUENCE SHOWN IN TABLE 11A

| | |
|---|---|
| MGSGRRALSAVPAVLLVLTLPGLPVWAQNDTEPIVLEGKCLVVCDSNPATDSKGSSSSPLGIVRAANSK | (SEQ ID NO:20) |
| VAFSAVRSTNHEPSEMSNKTRIIYFDQILVNVGNFFTLESVFVAPRKGIYSFSFHVIKVYQSQTIQVNLM | |
| LNGKPVISAFAGDKDVTREAATNGVLLYLDKEDKVYLKLEKGNLVGGWQYSTFSGFLVFPL | |

In a search of sequence databases, it was found, for example, that the nucleic acid sequence (SEQ ID NO: 19) has 392 of 602 bases (65%) identical to a Homo sapiens cerebellin mRNA (GENBANK-ID: HUMCERA|acc:M58583). The full amino acid sequence of the protein of the invention was found to have 146 of 195 amino acid residues (74%) identical to, and 171 of 195 residues (87%) positive with, the 224 amino acid residue cerebellin-like glycoprotein protein from rat (ptnr: SWISSPROT-ACC:P98087). The global sequence homology (as defined by GAP global sequence alignment with the full length sequence of this protein) is 77% amino acid similarity and 72% amino acid identity. In addition, this protein contains the c1q protein domain (IPR001073 as defined by Interpro) at amino acid positions 72 to 198. (Table 11C).

TABLE 11C

+HZ,55
BLASTX IDENTITY SEARCH AGAINST CEREBELLIN-LIKE GLYCOPROTEIN - RATTUS
norvegicus (RAT) (SEQ ID NO:38)

>ptnr: SWISSPROT-ACC:P98087 CEREBELLIN-LIKE GLYCOPROTEIN - Rattus
norvegicus (Rat), 224 aa.
Length = 224
Score = 727 (255.9 bits), Expect = 8.5e−72, P = 8.5e−72
Identities = 146/195 (74%), Positives = 171/195 (87%)

| | | | | |
|---|---|---|---|---|
| Query: | 7 | ALSAVPAVLLVLTLPGL-PVWAQNDTEPIVLEGKCLVVCDSNPATDSKGSSSSPLGISVR | 65 | |
| | | +\| \| \|+\|\|+\| \|\| \|\| \|\|\|\|\|\|\|\|\|\|\|\|\|\|\|\|\|\|\|\|+\|+ \| \|+ +\| \|\|\|\|\|\| | | |
| Sbjct: | 31 | SLGAALALLLLL-LPACCPVKAQNDTEPIVLEGKCLVVCDSSPSGD--GAVTSSLGISVR | 87 | |
| Query: | 66 | AANSKVAFSAVRSTNHEPSEMSNKTRIIYFDQILVNVGNFFTL-ESVFVAPRKGIYSFSF | 124 | |
| | | + ++\|\|\|\|\|\| \|\|\|\|\|\|\|\|\|\|\|\|+\| \|\|\|\|\|+\|\|\|+\|\| \| \| \|+\|\|\|\|\|\|\|\|\|\|\|\|\| | | |
| Sbjct: | 88 | SGSAKVAFSATRSTNHEPSEMSNRTMTIYFDQVLVNIGNHFDLASSIFVAPRKGIYSFSF | 147 | |
| Query: | 125 | HVIKVYQSQTIQVNLMLNGKPVISAFAGDKDVTREAATNGVLLYLDKEDKVYLKLEKGNL | 184 | |
| | | \|\|+\|\|\| \|\|\|\|\|+\|\| \|\| \|\|\|\|\|\|\|\|\|+\|\|\|\|\|\|\|+\|\|\|\|\| +++\|\|\|\|+\|\|\|\|+\|\|\| | | |
| Sbjct: | 148 | HVVKVYNRQTIQVSLMQNGYPVISAFAGDQDVTREAASNGVLLLMEREDKVHLKLERGNL | 207 | |
| Query: | 185 | VGGWQYSTFSGFLVFPL | 201 | (SEQ ID NO:20) |
| | | +\|\|\|+\|\|\|\|\|\|\|\|\|\|\|\| | | |
| Sbjct: | 208 | MGGWKYSTFSGFLVFPL | 224 | (SEQ ID NO:38) |

Other polypeptide sequences with homology to POLY10 are indicated in Table 11D.

TABLE 11D

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAY99402 Human PRO1382 (UNQ718) amino acid sequen . . . | +1 | 1020 | 3.9e−102 | 1 |
| patp:AAB66151 Protein of the invention #63 - Unidentif . . . | +1 | 1020 | 3.9e−102 | 1 |
| patp:AAY32937 Human cerebellin-2 protein sequence - Ho . . . | +1 | 728 | 3.4e−71 | 1 |
| patp:AAY01484 Cerebellin protein fragment (residues 64 . . . | +1 | 538 | 4.7e−51 | 1 |
| patp:AAW88747 Secreted protein encoded by gene 45 clon . . . | +1 | 511 | 3.4e−48 | 1 |
| patp:AAY99420 Human PRO1486 (UNQ755) amino acid sequen . . . | +1 | 509 | 5.6e−48 | 1 |

SignalP, Psort and/or hydropatahy suggest that POLY10 may be localized outside of the cell (Certainty=0.79) with a most likely cleavage site between positions 27 and 28 of SEQ ID NO:20. The predicted molecular weight is 21807.9 daltons.

Quantitative expression of POLY 10 was assessed as described in Example 4.

Immunoreactive cerebellin has been detected in every region of the brain studied, with the highest concentrations found in the hemisphere of the cerebellum and the vermis of the cerebellum. Immunoreactive cerebellin was also detected in the pituitary, the spinal cord and the normal parts of adrenal glands and some tumor tissues. Cerebellin proteins may have therapeutic applications in olivopontocerebellar atrophy (OPCA), Shy-Drager syndrome, ‘staggerer syndrome’ and various cancers such as, for example, brain and adrenal gland tumors, including phaeochromocytomas, cortisol-producing adrenocortical adenomas, ganglioneuroblastomas and neuroblastomas.

POLY11: Novel Lymphotactin-Like Protein and Nucleic Acids

Chemokines are a group of small (approximately 8 to 14 kD), mostly basic, structurally related molecules that regulate cell trafficking of various types of leukocytes through interactions with a subset of 7-transmembrane G protein-coupled receptors. Chemokines also play fundamental roles in the development, homeostasis, and function of the immune system, and they have effects on cells of the central nervous system as well as on endothelial cells involved in angiogenesis or angiostasis. Chemokines are divided into 2 major subfamilies, CXC and CC, based on the arrangement of the first 2 of the 4 conserved cysteine residues; the 2 cysteines are separated by a single amino acid in CXC chemokines and are adjacent in CC chemokines.

By screening a CD8+ T-lymphocyte cDNA library with a mouse lymphotactin probe, cDNAs encoding the lymphotactin XCL1, later designated SCYC1 were isolated. The sequence of the deduced 114-amino acid protein is most homologous to the CC chemokines CCL8 and CCL3, but differs in that it lacks the first and third cysteines characteristic of CC and CXC chemokines. By Northern blot analysis it was revealed that expression of an 0.8-kb SCYC1 transcript in activated thymic and peripheral blood CD8+ but not CD4+ T cells. In normal tissues, SCYC1 is expressed at high levels in spleen, thymus, small intestine, and peripheral blood leukocytes, as well as at low levels in lung, prostate, and ovary; it shows little or no expression in colon and testis. Lymphotactin is chemotactic for CD4+ and CD8+ T cells but not for monocytes, and induces a rise in intracellular calcium in peripheral blood lymphocytes.

Human Ltn shows similarity to some members of the C-C chemokine family but has lost the first and third cysteine residues that are characteristic of the C-C and C-X-C chemokines. Ltn is chemotactic for lymphocytes but not for monocytes, a characteristic that makes it unique among chemokines. In addition, calcium flux desensitization studies indicate that Ltn uses a unique receptor. The human Ltn gene maps to a different chromosome than do the C-C and C-X-C chemokine families. Taken together, these characteristics indicate that Ltn is the first example of a new class of human chemokines with preferential effects on lymphocytes.

From human PBMC stimulated with PHA, the present inventors have isolated cDNA clones encoding a novel cytokine named SCM-1, which is significantly related to the CC and the CXC chemokines but has only the 2nd and the 4th of the four cysteines conserved in these proteins. Its gene is also distinctly mapped to human chromosome 1. SCM-1 is strongly induced in human PBMC and Jurkat T cells by PHA stimulation. Among various human tissues, SCM-1 is expressed most strongly in spleen. SCM-1 is found to be 60.5% identical to lymphotactin, a recently described murine lymphocyte-specific chemokine, which also retains only two cysteines. SCM-1 and lymphotactin may thus represent the human and murine prototypes of a novel C or gamma type chemokine family.

The above defined information for this invention suggests that this lymphotactin-like protein may function as a member of a “Lymphotactin family”. Therefore, the novel nucleic acids and proteins identified here may be useful in potential therapeutic applications implicated in (but not limited to) various pathologies and disorders as a protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The POLY11 nucleic acids and proteins of the invention are thus useful in potential therapeutic applications implicated in development, homeostasis, and function of the immune system, and they have effects on cells of the central nervous system as well as on endothelial cells involved in angiogenesis or angiostasis and/or other pathologies and disorders. For example, a cDNA encoding the lymphotactin-like protein may be useful in gene therapy, and the lymphotactin-like protein may be useful when administered to a subject in need thereof. By way of nonlimiting example, the compositions of the present invention will have efficacy for treatment of patients suffering from CNS disorders, various types of cancer and immunological disorders. The novel nucleic acid encoding lymphotactin-like protein, and the lymphotactin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

A POLY11 nucleic acid that is comprised of 441 nucleotides (SEQ ID NO:21) was identified on chromosome 14. This POLY 11 nucleic acid encodes a lympotactin-like protein and is shown in Table 12A. An open reading frame was identified beginning with an ATG initiation codon at nucleotides 13-15 and ending with a TGA codon at nucleotides 421-423. The start and stop codons are in bold letters. Putative untranslated regions are located upstream from the initiation codon and downstream from the termination codon. The encoded protein having 136 amino acid residues (SEQ ID NO:22) is presented using the one-letter code in Table 12B.

TABLE 12A
The nucleotide sequence of POLY11.

(SEQ ID NO:21)

>GM87593525_A
CAGGAAACAAACATGGCCAACTTTTCTTACCGCTTCTCCATATACAACTTGAATGAAGCTCTGAATCAGGGAGAGACTGT
GGATCTGGATGCCTTGATGGCTGATCTTTGCTCTATAGAGCAGGAGCTCAGCAGCATTGGTTCAGGAAACAGTAAGCGTC
AAATCACAGAAACGAAACCTACTCAGAAATTGCCTGTTAGCCGACATACATTGAAACATGGCACCTTGAAAGGATTATCT
TCTTCATCTAATAGGATAGCTAAACCTTCCCATGCCAGCTACTCCTTGGACGACGTCACTGCACAGTTAGAACAGGCCTC
TTTGAGTATGGATGAGGCTGCTCAGCAATCTGTACTAGAAGATACTAAACCCTTAGTAACTAATCAGCACAGAAGAACCG
CAGTCAGCAGGCACAGTGAGTGATGCTGAAGTACACTCTAT

TABLE 12B
Protein sequence encoded by the coding sequence shown in TABLE 12A

(SEQ ID NO:22)

MANFSYRFSIYNLNEALNQGETVDLDALMADLCSIEQELSSIGSGNSKRQITETKATQKLPVSRHT
LKHGTLKGLSSSSNRIAKPSHASYSLDDVTAQLEQASLSMDEAAQQSVLEDTKPLVTNQHRRTAVSRHSE

In a search of CuraGen Corporation's proprietary human expressed sequence assembly database, assembly GM 87593625 (742 nucleotides) was identified as having 100% identity over 436 nucleotides to the presently identified gene sequence (SEQ ID NO:21). This database is composed of the expressed sequences (as derived from isolated mRNA) from more than 96 different tissues. The mRNA is converted to cDNA and then sequenced. These expressed DNA sequences are then pooled in a database and those exhibiting a defined level of homology are combined into a single assembly with a common consensus sequence. The consensus sequence is representative of all member components. Since the GM87593625_A nucleic acid of the present invention has 100% sequence identity with the CuraGen assembly, the nucleic acid of the invention represents an expressed gene sequence. This DNA assembly has 3 components and was found by CuraGen to be expressed in human lung tissue.

The full amino acid sequence of the protein of the invention was found to have 32 of 106 amino acid residues (30%) identical to, and 56 of 106 residues (52%) positive with, the 114 amino acid residue lymphotactin protein from Homo sapiens (ptnr:SPTREMBL-ACC:BAA09858) (Table 12C1). In addition it was found to have 31 of 106 amino acid residues (29%) identical to, and 56 of 106 residues (52%) positive with, the 114 amino acid residue lymphotactin protein from Homo sapiens (ptnr: SWISSNEW-ACC:P47992) (Table 12C2). The global sequence homology (as defined by FASTA alignment with the fill length sequence of this protein) is 25% amino acid identity and 31% amino acid similarity.

TABLE 12C

BLASTX identity search against SCM-1BETA PRECURSOR - Homo
sapiens (Human) (SEQ ID NO:39)

>ptnr:TREMBLNEW-ACC:BAA09858 SCM-1BETA PRECURSOR - Homo sapiens (Human), 114
aa.
Score = 82 (28.9 bits), Expect = 0.023, P = 0.023
Identities = 32/106 (30%), Positives = 56/106 (52%), Frame = +1

| | | | | |
|---|---|---|---|---|
| Query: | 91 | ALMADLCSIEQEL-SSIGSGNSKRQITETKATQKLPVSR---HTLKHGTLKGLSSSSNR- | 255 | (SEQ ID NO.22) |
| | | \|\|+ +\|\|+ + +\|\| \| \|+ + \|\|+\|\|\|\|\| +\|+ \|+\|+ + + \| | | |
| Sbjct: | 7 | ALLG-ICSLTAYIVEGVCSEVSHRRTCVSLTTQRLPVSRIKTYTITEGSLPAVIFITKRG | 65 | (SEQ ID NO:39) |
| Query: | 256 | --IAKFSHASYSLDDVTAQLEQASLSMDEAAQQSVLEDTKPLVTNQERRTAVS 408 | | |
| | | + \|++ + \|\| +++ \| + + \| \|\|\| \| \| \|\|\|+ | | |
| Sbjct: | 66 | LKVCADPQATW-VRDVVRSMDRKSNTRNNMIQ------TKPTGTQQSTNTAVT 111 | | |

TABLE 12D

BLASTX identity search against LYMPHOTACTIN PRECURSOR
(CYTOKINE SCM-1)(ATAC)(LYMPHOTAXIN)(SCM-1- ALPHA)(SMALL
INDUCIBLE CYTOKINE C1) - Homo sapiens (Human) (SEQ ID NO:40)

>ptnr:SWISSNEW-ACC:P47992 LYMPHOTACTIN PRECURSOR (CYTOKINE SCM-1) (ATAC)
(LYMPHOTAXIN) (SCM-1- ALPHA) (SMALL INDUCIBLE CYTOKINE C1) - Homo sapiens
(Human), 114 aa.
Score = 79 (27.8 bits), Expect = 0.16, P = 0.15
Identities = 31/106 (29%), Positives = 56/106 (52%), Frame = + 1

| | | | | |
|---|---|---|---|---|
| Query: | 91 | ALMADLCSIEQEL-SSIGSGNSKRQITETKATQKLPVSR---HTLKHGTLKGLSSSSNR- | 255 | (SEQ ID NO.22) |
| | | \|\|+ +\|\|+ + +\|\| \| ++ + \|\|+\|\|\|\|\| +\|+ \|+\|+ + + \| | | |
| Sbjct: | 7 | ALLG-ICSLTAYIVEGVGSEVSDKRTCVSLTTQRLPVSRIKTYTITEGSLRAVIFITKRG | 65 | (SEQ ID NO:40) |
| Query: | 256 | --IAKPSHASYSLDDVTAQLEQASLSMDEAAQQSVLEDTKPLVTNQHRRTAVS 408 | | |
| | | + \|++ + \|\| +++ \| + + \| \|\|\| \| \| \|\|\|+ | | |
| Sbjct: | 66 | LKVACDPQATW-VRDVVRSMDRKSNTRNNMIQ------RKPTGTQQSTNTAVT 111 | | |

TABLE 12E

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAP81169 Protein produced in myeloma cell differe . . . | +1 | 167 | 9.7e−12 | 1 |
| patp:AAW14281 Human neuroblastoma-specific thymosin-be . . . | +1 | 161 | 4.2e−11 | 1 |
| patp:AAWB1508 Thymosin beta 4, Y isoform (TB4Y) gene p . . . | +1 | 159 | 6.8e−11 | 1 |
| patp:AAW81507 Thymosin beta 4, X isoform (TB4X) gene p . . . | +1 | 158 | 8.7e−11 | 1 |
| patp:AAB53712 Human colon cancer antigen protein seque . . . | +1 | 158 | 8.7e−11 | 1 |
| patp:AAY91956 Human cytoskeleton associated protein 11 . . . | +1 | 158 | 8.7e−11 | 1 |
| patp:AAW46486 Human thymosin beta-15 protein - Homo sa . . . | +1 | 156 | 1.4e−10 | 1 |
| patp:AAW36056 Human thymosin beta-15 protein sequence . . . | +1 | 154 | 2.3e−10 | 1 |
| patp:AAW68573 Rat thymosin-beta15 protein - Rattus sp, . . . | +1 | 154 | 2.3e−10 | 1 |
| patp:AAW44275 Human thymosin beta 15 - Homo sapiens, 4 . . . | +1 | 154 | 2.3e−10 | 1 |

Psort analysis predicts that POLY11 may be localized in the cytoplasm with a certainty of 0.65. Using the SignalP analysis, no signal peptide was identified. The predicted molecular weight is 14934.4 daltons.

Quantitative expression of POLY11 was assessed as described in Example 4.

POLY11 is a novel member of the lymphotactin-like family of proteins, and is thus useful to identify lyphotactin-like protein-binding proteins. The lymphotactin-like family proteins are a class of lymphocyte-specific chemokine, which POLY11 is useful in potential therapeutic applications implicated in development, homeostasis, and function of the immune system. Also, POLY11 as a lymphotactin-like protein is useful in diagnostic or therapeutic applications to pathologies also have effects on cells of the central nervous system as well as on endothelial cells involved in angiogenesis or angiostasis and/or other pathologies and disorders.

POLY12: Novel Zinc Transporter-Like Protein and Nucleic Acids

Genes that are involved in mammalian zinc transport recently have been cloned. These genes predict proteins with multiple membrane spanning regions, and most have a histidine-rich intracellular loop. ZnT-1 was the first cloned and is associated with zinc efflux. It is found in all tissues examined, and, at least in some, ZnT-1 expression is regulated by dietary zinc intake. In enterocytes of the small intestine and renal tubular cells, ZnT-1 is localized to the basolateral membrane, suggesting an orientation that is consistent with zinc absorption/retention. ZnT-2 is also an exporter and may be involved in zinc efflux or uptake into vesicles in intestine, kidney, and testis. ZnT-3 is involved in zinc uptake into vesicles in neurons and possibly in testis. ZnT-4 is also an exporter and is highly expressed in mammary gland and brain. The divalent cation transporter 1 (DCT1) is regulated by iron, but exhibits transport activity for a number of trace elements including zinc. Description of a family of zinc transporters bridges the integrative and reductionist approach to the study of zinc metabolism. Other members of this transporter family may emerge. Many of these may be regulated by zinc, and some may respond to immune challenge, oxidative stress, and competing metals in the dietary supply. Collectively, description of transporters that influence cellular zinc uptake and efflux will provide a clearer understanding of the molecular events that regulate zinc absorption and homeostasis.

Zinc is the second most abundant trace metal in the human body. It is an essential element, serving both a structural role, as in the formation of zinc fingers in DNA-binding proteins, and a catalytic role in metalloenzymes, such as pancreatic carboxypeptidases (e.g., 114852), alkaline phosphatases (e.g., 171760), various dehydrogenases, and superoxide dismutases (e.g., 147450).The valence stability of zinc, its lack of direct toxicity, and its coordination flexibility make it an ideal metal in carrying out these diverse biologic functions. A family of related zinc-transport proteins (symbolized Znt1, Znt2, and Znt3 (602878) by them) have been found in mammals. In general, the ZNT proteins are predicted to have similar structures, consisting of 6 transmembrane domains and a histidine-rich cytoplasmic loop, and they appear to transport zinc out of the cytoplasm. Znt1 appears to be expressed ubiquitously, and the protein, which resides on the plasma membrane, confers zinc resistance to zinc-sensitive cell lines, presumably by direct export of zinc out of the cell. Znt2 and Znt3 are more similar to each other than they are to Znt1 and are located on vesicular membranes. Znt2 has been implicated in zinc accumulation in endosomal/lysosomal compartments, and association of Znt3 with synaptic vesicles of the brain suggests that it is intimately involved in zinc incorporation into these structures.

The 429-amino acid human ZNT4 polypeptide shares 92% predicted identity with the mouse gene. It follows from available information on conservation of synteny that the gene encoding ZNT4 in human is almost certainly located on chromosome 20 in the general vicinity of the human homolog of agouti (600201), which is situated at 20q11.2.

Most zinc deficiencies in humans are dietary inadequacies; however, instances of zinc deficiency stemming from inadequate zinc content of milk in full term breastfed babies have been reported. Transient zinc deficiency in 2 full term breastfed sibs that could be related to low maternal breast milk zinc concentration has been reported. In contrast to the ability to rescue the lethal milk phenotype in mice by maternal zinc administration, oral supplementation of zinc to human mothers with low zinc content of their milk did not lead to an increased zinc content. Perhaps the only inherited disorder in humans that has been related to a primary defect in zinc transport is acrodermatitis enteropathica (201100). This rare, autosomal recessive disorder results from a defect in intestinal absorption of zinc and manifests with a variety of findings similar to those in dietary zinc deficiency, such as periorificial dermatitis, diarrhea, alopecia, growth retardation, and susceptibility to infections, with or without neuropsychiatric involvement. Since the mid-1970s, patients with acrodermatitis enteropathica have been effectively treated with daily zinc supplementation. Of interest would be the development of utricular otoliths in these patients. Patients are now living to childbearing age; the therapeutic regimen might supplement any zinc deficiency in milk from acrodermatitis enteropathica mothers and mask a maternal effect.

Another murine milk deficiency, ‘toxic milk’ (tx), is due to a fatal deficiency of copper in the milk of mutant dams and has its genetic basis in a mutation in the murine homolog of the Wilson disease gene (277900). Like Wilson patients, the toxic milk animals have copper accumulation in the liver, but copper deficiency in human milk has not, it seems, been found in Wilson disease. Thus there are substantially different physiologic consequences, owing to the absence of this copper transporter in these 2 species; the same might be true for a zinc transporter.

A novel nucleic acid that is comprised of 1665 nucleotides (SEQ ID NO: 23) was identified on chromosome 15. This POLY 12 nucleic acid encodes a zinc transporter like protein and is shown in Table 13A. An open reading frame was identified beginning with an ATG initiation codon at nucleotides 77-79 and ending with a TGA codon at nucleotides 1598-1600. The start and stop codons are in bold letters. A putative untranslated region was found upstream from the initiation codon and downstream from the termination codon. The encoded protein having 512 amino acid residues (SEQ ID NO: 24) is presented using the one-letter code in Table 13B.

TABLE 13A
The nucleotide sequence of POLY12.

(SEQ ID NO:23)

>GM87756960_A
CGACCCTCCGCGTCCCGCCAACGCCGCCGCTGCACCAGTCTCCGGGCCGGGCTCGGCGGGCCCCGCAGCCGCAGCC
ATGGGGTGTTGGGGTCGGAACCGGGGCCGGCTGCTGTGCATGCTGGCGCTGACCTTCATGTTCATGGTGCTGGAGG
TGGTGGTGAGCCGGGTGACCTCGTCGCTGGCGATGCTCTCCGACTCCTTCCACATGCTGTCGGACGTGCTGGCGCT
GGTGGTGGCGCTGGTGGCCGAGCGCTTCGCCCGGCGGACCCACGCCACCCAGAAGAACACGTTCGGCTGGATCCGA
GCCGAGGTAATGGGGGCTCTGGTGAACGCCATCTTCCTGACTGGCCTCTGTTTCGCCATCCTGCTGGAGGCCATCG
AGCGCTTCATCGAGCCGCACGAGATGCAGCAGCCGCTGGTGGTCCTTGGGGTCGGCGTGGCCGGGCTGCTGGTCAA
CGTGCTGGGGCTCTGCCTCTTCCACCATCACAGCGGCTTCAGCCAGGACTCCGGCCACGGCCACTCGCACGGGGGT
CACGGCCACGGCCACGGCCTCCCCAAGGGGCCTCGCGTTAAGAGCACCCGCCCCGGGAGCAGCGACATCAACGTGG
CCCCGGGCGAGCAGGGTCCCGACCAGGAGGAGACCAACACCCTGGTGGCCAATACCAGCAACTCCAACGGGCTGAA
ATTGGACCCCGCGGACCCAGAAAACCCCAGAAGTGGTGATACAGTGGAAGTACAAGTGAATGGAAATCTTGTCAGA
GAACCTGACCATATGGAACTGGAAGAAGATAGGGCTGGACAACTTAACATGCGTGGAGTTTTTCTGCATGTCCTTG
GAGATGCCTTGGGTTCAGTGATTGTAGTAGTAAATGCCTTAGTCTTTTACTTTTCTTGGAAAGGTTGTTCTGAAGG
GGATTTTTGTGTGAATCCATGTTTCCCTGACCCCTGCAAAGCATTTGTAGAAATAATTAATAGTACTCATGCATCA
GTTTATGAGGCTGGTCCTTGCTGGGTGCTATATTTAGATCCAACTCTTTGTGTTGTAATGGTTTGTATACTTCTTT
ACACAACCTATCCATTACTTAAGGAATCTGCTCTTATTCTTCTACAAACTGTTCCTAAACAAATTGATATCAGAAA
TTTGATAAAAGAACTTCGAAATGTTGAAGGAGTTGAGGAAGTTCATGAATTACATGTTTGGCAACTTGCTGGAAGC
AGAATCATTGCCACTGCTCACATAAAATGTGAAGATCCAACATCATACATGGAGGTGGCTAAAACCATTAAAGACG
TTTTTCATAATCACGGAATTCACGCTACTACCATTCAGCCTGAATTTGCTAGTGTAGGCTCTAAATCAAGTGTAGT
TCCGTGTGAACTTGCCTGCAGAACCCAGTGTGCTTTGAAGCAATGTTGTGGGACACTACCACAAGCCCCTTCTGGA
AAGGATGCAGAAAAGACCCCAGCAGTTAGCATTTCTTGTTTAGAACTTAGTAACAATCTAGAGAAGAAGCCCAGGA
GGACTAAAGCTGAAAACATCCCTGCTGTTGTGATAGAGATTAAAAACATGCCAAACAAACAACCTGAATCATCTTT
GTGAGTCTTGAAAAAGATGTGATATTTGACTTTTGCTTTAAACTGCAAGAGGAAAAAGACTCCACTGAA

TABLE 13B
Protein sequence encoded by the coding sequence shown in TABLE 13A

(SEQ ID NO: 24)

MGCWGRNRGRLLCMLALTFMFMVLEVVVSRVTSSLAMLSDSFHMLSDVLALVVALVAERFARRTHATQKNTFGWIR
AEVMGALVNAIFLTGLCFAILLEAIERFIEPHEMQQPLVVLGVGVAGLLVNVLGLCLFHHHSGFSQDSGHGHSHGG
HGHGHGLPKGPRVKSTRPGSSDINVAPGEQGPDQEETNTLVANTSNSNGLKLDPAGEPGKDPENPRSGDTVEVQVN
GNLVREPDHMELEEDRAGQLNMRGVFLHVLGDALGSVIVVVNALVFYFSWKGCSEGDFCVNPCFPDPCKAFVEIIN
STHASVYEAGPCWVLYLDPTLCVVMVCILLYTTYPLLKESALILLQTVPKQIDIRNLIKELRNVEGVEEVHELHVW
QLAGSRIIATAHIKCEDPTSYMEVAKTIKDVFHNHGIHATTIQPEFASVGSKSSVVPCELACRTQCALKQCCGTLP
QAPSGKDAEKTPAVSISCLELSNNLEKKPRRTKAENIPAVVIEIKNMPNKQPESSL

In a search of sequence databases, it was found, for example, that the nucleic acid sequence (SEQ ID NO:23) has 1198 of 1629 bases (73%) identical to a Rattus norvegicus zinc transporter mRNA (GENBANK-ID: U17133). In addition it was found that the nucleic acid sequence has 100% of 359 bases identical to a human zinc transporter mRNA (GENBANK-ID:AF048701|acc:AF048701). The full amino acid sequence of the protein of the invention was found to have 438 of 512 amino acid residues (85%) identical to, and 468 of 512 residues (91%) positive with, the 507 amino acid residue zinc transporter-1 protein from Rattus norvegicus (ptnr:SPTREMBL-ACC:Q62720) (Table 13C). The global sequence homology (as defined by GAP global sequence alignment with the full length sequence of this protein) is 88% amino acid similarity and 85% amino acid identity. In addition, this protein contains the Cation efflux family domain (IPR002524 as defined by Interpro) at amino acid positions 65 to 428.

TABLE 13C
BLASTX identity search against Rattus norvegicus (Rat) (SEQ ID NO:41)

>ptnr:SPTREMBL-ACC:Q62720 ZNT-1 - Rattus norvegicus (Rat), 507 aa.
Score = 2253 (793.1 bits), Expect = 1.0e−232, P = 1.0e−232
Identities = 438/512 (85%), Positives = 468/512 (91%), Frame = +2

| | | | | |
|---|---|---|---|---|
| Query: | 77 | MGCWGRNRGRLLCMLALTFMFMVLEVVVSRVTSSLAMLSDSFHMLSDVLALVVALVAERF | 256 | (SEQ ID NO:24) |
| | | \|\|\|\|\|\|\|\|\|\|\|\|\|\|\| \|\|\|\|\|\|\|\|\|\|\|\|\|\|\|\|+\|\|\|\|\|\|\|\|\|\|\|\|\|\|\|\|\|\|\|\|\|\|\|\|\|\|\| | | |
| Sbjct: | 1 | MGCWGRNRGRLLCMLLLTFMFMVLEVVVSRVTASLAMLSDSFHMLSDVLALVVALVAERF | 60 | (SEQ ID NO:41) |
| Query: | 257 | ARRTHATQKNTFGWIRAEVMGALVNAIFLTGLCFAILLEAIERFIEPHEMQQPLVVLGVG | 436 | |
| | | \|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\| \|\| | | |
| Sbjct: | 61 | ARRTHATQKNTFGWIRAEVMGALVNAIFLTGLCFAILLEAVERFIEPHEMQQPLVVLSVG | 120 | |
| Query: | 437 | VAGLLVNVLGLCLFHHHSGFSQDSGHGHSHGGHGHGHGLPKGPRVKSTRPGSSDINVAPG | 616 | |
| | | \|\|\|\|\|\|\|\|\|\|\|\|\|\|\|\|\|\|\| \| +\|\|\|\|\|\|\| \|\|\|\|\| \| \|\| \| \|+ \| \| + \|\| | | |
| Sbjct: | 121 | VAGLLVNVLGLCLFHHHSGEGQGAGHGHSHG-HGHGH-LAKGAR-KAGRAGG-EAGAPPG | 176 | |
| Query: | 617 | ---EQGPDQEETNTLVANTSNSNGLKLDPAGEPGKDPENPRSGDTVEVQVNGNLVREPDH | 787 | |
| | | +\| \|\|\|\|\|\|\|\|\|\|\|\|\|\|\|\|\|\|\|\| \| \| +\|\| \|\| \| \|+\|\|\|\|\|\|\|++\| \| | | |
| Sbjct: | 177 | RAPDQEPDQEETNTLVANTSNSNGLKADQA-----EPEKLRSDDPVDVQVNGNLIQESDS | 231 | |
| Query: | 788 | MELEEDRAGQLNMRGVFLHVLGDALGSVIVVVNALVFYFSWKGCSEGDFCVNPCFPDPCK | 967 | |
| | | +\| \|++\|\|\|\|\|\|\|\|\|\|\|\|\|\|\|\|\|\|\|\|\|\|\|\|\|\|\|\|\|\|\|\|\|\|\|\|\|\|+\| \|\|\|\|\|\|\|\|\|\|\|\|\| | | |
| Sbjct: | 232 | LESEDNRAGQLNMRGVFLHVLGDALGSVIVVVNALVFYFSWKGCTEDDFCVNPCFPDPCK | 291 | |
| Query: | 968 | AFVEIINSTHASVYEAGPCWVLYLDPTLCVVMVCILLYTTYPLLKESALILLQTVPKQID | 1147 | |
| | | + \|\|++\|\|\| \| ++\|\|\|\|\|\|\|\|\|\|\|\|\|\|\|++\|\|\|\|\|\|\|\|\|\|\|\|\|\|\|\|\|\|\|\|\|\|\|\|\|\|\|\|\| | | |
| Sbjct: | 292 | SSVELMNSTQAPMHEAGPCWVLYLDPTLCIIMVCILLYTTYPLLKESALILLQTVPKQID | 351 | |
| Query: | 148 | IRNLIKELRNVEGVEEVHELHVWQLAGSRIIATAHIKCEDPTSYMEVAKTIKDVFHNHGI | 1327 | |
| | | \|++\|+\|\|\|\|+\|\|\|\|\|\|\|\|\|\|\|\|\|\|\|\|\|\|\|\|\|\|\|\|\|\|\|\|\|\|\| \|\|\|+\|\|\|\|\|\|\|\|\|\|\|\|\|\| | | |
| Sbjct: | 352 | IKHLVKELRDVEGVEEVHELHVWQLAGSRIIATAHIKCEDPASYMQVAKTIKDVFHNHGI | 411 | |
| Query: | 1328 | HATTIQPEFASVGSKSSVVPCELACRTQCALKQCCGTLPQAPSGKDAEKTPAVSISCLEL | 1507 | |
| | | \|\|\|\|\|\|\|\|\|\|\|\|\|\|\|\|\|\|\|\|\|\|\|\|\|\|\|\|\|\|\|\|\|\|\|\|\| \|\| \|\|\|+\|\|\| \| \|\|\|\|\|\|\|\| | | |
| Sbjct: | 412 | HATTIQPEFASVGSKSSVVPCELACRTQCALKQCCGTRPQVHSGKEAEKAPTVSISCLEL | 471 | |
| Query: | 1508 | SNNLEKKPRRTKAE-NIPAVVIEIKNMPNKQPESSL 1612 | | |
| | | \| \|\|\|\|\|\|\|\|\|\|\|\| ++\|\|\|\|\|\|\|\|\|+\|\|\|\|\|\|\|\|\| | | |
| Sbjct: | 472 | SENLEKKPRRTKAEGSVPAVVIEIKNVPNKQPESSL 507 | | |

TABLE 13D

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAY86241 Human secreted protein HOABR60, SEQ ID N . . . | +2 | 2529 4.9e−262 | 1 | |
| patp:AAY8E410 Human gene 27-encoded protein fragment, . . . | +2 | 1526 1.5e−158 | 2 | |
| patp:AAY86316 Human secreted protein HOABR60, SEQ ID N . . . | +2 | 1482 6.7e−154 | 2 | |
| patp:AAR04584 Protein product of plasmid pEN10 contain . . . | +2 | 447 | 3.5e−64 | 2 |
| patp:AAR95451 Yeast OSR - Saccharomyces cerevisiae str . . . | +2 | 447 | 3.5e−64 | 2 |
| patp:AAY12709 Human 5′ EST secreted protein SEQ ID NO: . . . | +2 | 474 | 3.5e−58 | 2 |
| patp:AAG22264 Arabidopsis thaliana protein fragment SE . . . | +2 | 245 | 1.2e−34 | 3 |

PSORT analysis suggests that the protein may be localized in the plasma membrane with a certainty of 0.6400. Using SignalP analysis, it is predicted that POLY12 has a signal peptide with most likely cleavage site between residues 29 and 30: VVS-RV (SEQ ID NO:24). The predicted molecular weight is 55767.8 daltons.

Quantitative expression of POLY12 was assessed as described in Example 4.

The zinc transporter proteins are implicated in the transport of zinc, an important trace, metal, in organisms with zinc deficiencies. The zinc transporter proteins are thus useful in potential therapeutic applications implicated in disorders related to zinc deficiencies including immune challenge, oxidative damage, dermatitis, alopecia, stunted growth or deficiencies of varying levels of other metals that compete for these transporters.

POLY13: Novel Macrophage-Stimulating Protein Precursor-Like Protein and Nucleic Acids

Macrophage stimulating protein and hepatocyte growth factor stand out from other cytokines because of their uncommon biological properties. In addition to promoting cell growth and protection from apoptosis, they are involved in the control of cell dissociation, migration into extracellular matrices, and a unique process of differentiation called ‘branching morphogenesis’. Through the concerted regulation of these complex phenomena, macrophage stimulating proteins promote development, regeneration, and reconstruction of normal organ architecture. In transformed epithelia, macrophage stimulating proteins can mediate tumor invasive growth, a harmful feature of neoplastic progression in which cancer cells invade surrounding tissues, penetrate across the vascular walls, and eventually disseminate throughout the body, giving rise to systemic metastases. A much-debated issue in basic biology, which has strong implications for experimental medicine, is how to dissociate the favorable effects of growth factors from their adverse ones. Accordingly, to find agonists or antagonists with potential therapeutic applications is a crucial undertaking for current research. Domain-mapping analyses of growth factor molecules can help to isolate specific structural requirements for the induction of selective biological effects. Based on the observation that certain growth factors must undergo posttranslational modifications to exert a full response, it is possible to interfere with their activation mechanisms to modulate their functions. Finally, the identification of cell type-specific coreceptors able to potentiate their activity allows drawing of a functional body map, where some organs or tissues may be more responsive than others to growth factors.

A novel nucleic acid that is comprised of 2200 nucleotides (SEQ ID NO:25) was identified on chromosome 1. This POLY13 nucleic acid encodes a macrophage stimulating protein (MSP) precursor-like protein is shown in Table 14A. An open reading frame was identified beginning with an ATG initiation codon at nucleotides 21-23 and ending with a TAG codon at nucleotides 2157-2159. The start and stop codons are in bold letters. A putative untranslated region was found upstream from the initiation codon and downstream from the termination codon. The encoded protein having 712 amino acid residues (SEQ ID NO:26) is presented using the one-letter code in Table 14B.

TABLE 14A
The nucleotide sequence of POLY13.

| | |
|---|---|
| >GM105274478_A | (SEQ ID NO:25) |

TGCAGCCTCCAGCCAGAAGGATGGGGTGGCTCCCACTCCTGCTGCTTCTGACTCAATGCTTAGGGGTCC
CTGGGCAGCGCTCGCCATTGAATGACTTCGAGGTGCTCCGGGGCACAGAGCTACAGCGGCTGCTACAAG
CGGTGGTGCCCGGGCCTTGGCAGGAGGATGTGGCAGATGCTGAAGAGTGTGCTGGTCGCTGTGGGCCCT
TAATGGACTGCCGGGCGTTCCACTACAATGTGAGCAGCCATGGTTGCCAACTGCTGCCATGGACTCAAC
ACTCACCCCACACGAGGCTGCGGCATTCTGGGCGCTGTGACCTCTTCCAGGAGAAAGACTACATACGGA
CCTGCATCATGAACAATGGGGTTGGGTACCGGGGCACCATGGCCACGACCGTGGGTGGCCTGTCCTGCC
AGGCTTGGAGCCACAAGTTCCCGAACGATCACAGGTACATGCCCACGCTCCGGAATGGCCTGGAAGAGA
ACTTCTGCCGTAACCCTGATGGCGACCCCGGAGGTCCTTGGTGCCACACAACAGACCCTGCCGTGCGCT
TCCAGAGCTGCGGCATCAAATCCTGCCGGTCTGCCGCGTGTGTCTGGTGCAATGGCGAGGAATACCGCG
GCGCGGTAGACCGCACCGAGTCAGGGCGCGAGTGCCAGCGCTGGGATCTTCAGCACCCGCACCAGCACC
CCTTCGAGCCGGGCAAGTACCCCGACCAAGGTCTGGACGACAACTATTGCCGGAATCCTGACGGCTCCG
AGCGGCCATGGTGCTACACTACGGATCCGCAGATCGAGCGAGAATTCTGTGACCTCCCCCGCTGCGGTT
CCGAGGCACAGCCCCGCCAAGAGGCCACAAGTGTCAGCTGCTTCCGCGGGAAGGGTGAGGGCTACCGGG
GCACAGCCAATACCACCACCGCGGGCGTACCTTGCCAGCGTTGGGACGCGCAAATCCCGCATCAGCACC
GATTTACGCCAGAAAAATACGCGTGCAAGGACCTTCGGGAGAACTTCTGCCGGAACCCCGACGGCTCAG
AGGCGCCCTGGTGCTTCACACTGCGGCCCGGCATGCGCGTGGGCTTTTGCTACCAGATCCGGCGTTGTA
CAGACGACGTGCGGCCCCAGGGTTGCTACCACGGCGCGGGGGAGCAGTACCGCGGCACGGTCAGCAAGA
CCCGCAAGGGTGTCCAGTGCCAGCGCGCGTCCGCTGAGACGCCGCACAAGCCGCAGTTTACCTTTACCT
CCGAACCGCATGCACAACTGGAGGAGAACTTCTGCCGCGACCCAGATGGGGATAGCTATGGGCCCTGGT
GCTACACGATGGACCCAAGGACCCCATTCGACTACTGTGCCCTGCGACGCTGCGCTGATGACCAGCCGC
CATCAATCCTGGACCCCCCCGACCAGGTGCAGTTTGAGAAGTGTGGCAAGAGGGTGGATCGGCTGGATC
AGCGTTGTTCCAAGCTGCGCGTGGCTGGGGGCCATCCGGGCAACTCACCCTGGACAGTCAGCTTGCGGA
ATAGGCAGGGCCAGCATTTCTGCGGGGGGTCTCTAGTGAAGGAGCAGTGGATACTGACTGCCCGGCAGT
GCTTCTCCTCCAGCCATATGCCTCTCACGGGCTATGAGGTATGGTTGGGCACCCTGTTCCAGAACCCAC
AACATGGAGAGCCAGGCCTACAGCGGGTCCCAGTAGCCAAGATGCTGTGTGGGCCCTCAGGCTCTCAGC
TTGTCCTGCTCAAGCTGGAGAGATCTGTGACCCTGAACCAGCGTGTGGCCCTGATCTGCCTGCCGCCTG
AATGGTATGTGGTGCCTCCAGGGACCAAGTGTGAGATTGCAGGCCGGGGTGAGACCAAAGGTACGGGTA
ATGACACAGTCCTAAATGTGGCCTTGCTGAATGTCATCTCCAACCAGGAGTGTAACATCAAGCACCGAG
GACATGTGCGGGAGAGCGAGATGTGCACTGAGGGACTGTTGGCCCCTGTGGGGGCCTGTGAGGGGGGTG
ACTACGGGGGCCCACTTGCCTGCTTTACCCACAACTGCTGGGTCCTGGAAGGAATTAGAATCCCCAACC
GAGTATGCGCAAGGTCGCGCTGGCCAGCCGTCTTCACACGTCTCTCTGTGTTTGTGGACTGGATTCACA
AGGTCATGAGACTGGGTTAGGCCCAGCCTTGACGCCATATGCTTTGGGGAGGACAAAACTT

TABLE 14B
Protein sequence encoded by the coding sequence shown in TABLE 14A

(SEQ ID NO:26)
MGWLPLLLLLTQCLGVPGQRSPLNDFEVLRGTELQRLLQAVVPGPWQEDVADAEECAGRCGPLMDCRAF
HYNVSSHGCQLLPWTQHSPHTRLRHSGRCDLFQEKDYIRTCIMNNGVGYRGTMATTVGGLSCQAWSHKF
PNDHRYMPTLRNGLEENFCRNPDGDPGGPWCHTTDPAVRFQSCGIKSCRSAACVWCNGEEYRGAVDRTE
SGRECQRWDLQHPHQHPFEPGKYPDQGLDDNYCRNPDGSERPWCYTTDPQIEREFCDLPRCGSEAQPRQ
EATSVSCFRGKGEGYRGTANTTTAGVPCQRWDAQIPHQHRFTPEKYACKDLRENFCRNPDGSEAPWCFT
LRPGMRVGFCYQIRRCTDDVRPQGCYHGAGEQYRGTVSKTRKGVQCQRASAETPHKPQFTFTSEPHAQL
EENFCRDPDGDSYGPWCYTMDPRTPFDYCALRRCADDQPPSILDPPDQVQFEKCGKRVDRLDQRCSKLR
VAGGHPGNSPWTVSLRNRQGQHFCGGSLVKEQWILTARQCFSSSHMPLTGYEVWLGTLFQNPQHGEPGL
QRVPVAKMLCGPSGSQLVLLKLERSVTLNQRVALICLPPEWYVVPPGTKCEIAGRGETKGTGNDTVLNV
ALLNVISNQECNIKHRGHVRESEMCTEGLLAPVGACEGGDYGGPLACFTHNCWVLEGIRIPNRVCARSR
WPAVFTRVSVFVDWIHKVMRLG

In a search of sequence databases, it was found, for example, that the nucleic acid sequence (SEQ ID NO:25) has 2132 of 2203 bases (96%) identical to a Homo sapiens macrophage-stimulating protein mRNA (GENBANK-ID: L11924). The full amino acid sequence of the protein of the invention was found to have 682 of amino acid residues (95%) identical to, and 692 of 712 residues (97%) positive with, the 711 amino acid residue MACROPHAGE-STIMULATING PROTEIN PRECURSOR-protein from Homo sapiens (ptnr:SPTREMBL-ACC: Q14870) (Table 14C). The global sequence homology (as defined by GAP global sequence alignment with the full length sequence of this protein) is 97% amino acid similarity and 95% amino acid identity.

TABLE 14C

BLASTX identity search against MACROPHAGE-STIMULATING
PROTEIN PRECURSOR- Homo sapiens (Human)(SEQ ID NO:42)

>ptnr:SPTREMBL-ACC:Q14870 MACROPHAGE-STIMULATING PROTEIN PRECURSOR- Homo sapiens
(Human), 711 aa.
Score = 3871 (1362.7 bits), Expect = 0.0, P = 0.0
Identities = 682/712 (95%), Positives = 692/712 (97%), Frame = + 2

| | | | | |
|---|---|---|---|---|
| Query: | 29 | MGWLPLLLLLTQCLGVPGQRSPLNDFEVLRGTELQRLLQAVVPGPWQEDVADAEECAGRC | 208 | (SEQ ID NO.26) |
| | | \|\|\|\|\|\|\|\|\|\|\|\|\|\|\|\|\|\|\|\|\|\|\|\|\|\|+\|\|\|\|\|\|\|\| \|\| \|\|\|\|\|\|\|\|\|\|\|\|\|\|\|\|\| | | |
| Sbjct: | 1 | MGWLPLLLLLTQCLGVPGQRSPLNDFQVLRGTELQHLLHAVVPGPWQEDVADAEECAGRC | 60 | (SEQ ID NO:42) |
| Query: | 209 | GPLMDCRAFHYNVSSHGCQLLPWTQHSPHTRLRHSGRCDLFQEKDYIRTCIMNNGVGYRG | 388 | |
| | | \|\|\|\|\|\|\|\|\|\|\|\|\|\|\|\|\|\|\|\|\|\|\|\|\|\|\|\|\|\|\|\|\| \|\|\|\|\|\|\|\|+\|\|\|+\|\|\|\|\|\|\|\|\|\|\|\|\| | | |
| Sbjct: | 61 | GPLMDCRAFHYNVSSHGCQLLPWTQHSPHTRLRRSGRCDLFQKKDYVRTCIMNNGVGYRG | 120 | |
| Query: | 389 | TMATTVGGLSCQAWSHKFPNDHRYMPTLRNGLEENFCRNPDGDPGGPWCHTTDPAVRFQS | 568 | |
| | | \|\|\|\|\|\|\|\|\| \|\|\|\|\|\|\|\|\|\|\|\|+\| \|\|\|\|\|\|\|\|\|\|\|\|\|\|\|\|\|\|\|\|\|\|\|\|+\|\|\|\|\|\|\|\|\|\| | | |
| Sbjct: | 121 | TMATTVGGLPCQAWSHKFPNDHKYTPTLRNGLEENFCRNPDGDPGGPWCYTTDPAVRFQS | 180 | |
| Query: | 569 | CGIKSCRSAACVWCNGEEYRGAVDRTESGRECQRWDLQHPHQHPFEPGKYPDQGLDDNYC | 748 | |
| | | \|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|+ \|\|\|\|\|\|\|\|\| | | |
| Sbjct: | 181 | CGIKSCREAACVWCNGEEYRGAVDRTESGRECQRWDLQHPHQHPFEPGKFLDQGLDDNYC | 240 | |
| Query: | 749 | RNPDGSERPWCYTTDPQIEREFCDLPRCGSEAQPRQEATSVSCFRGKGEGYRGTANTTTA | 928 | |
| | | \|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\| | | |
| Sbjct: | 241 | RNPDGSERPWCYTTDPQIEREFCDLPRCGSEAQPRQEATTVSCFRGKGEGYRGTANTTTA | 300 | |
| Query: | 929 | GVPCQRWDAQIPHQHRFTPEKYACKDLRENFCRNPDGSEAPWCFTLRPGMRVGFCYQIRR | 1108 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\| | | |
| Sbjct: | 301 | GVPCQRWDAQIPHQHRFTPEKYACKDLRENFCRNPDGSEAPWCFTLRPGMRAAFCYQIRR | 360 | |
| Query: | 1109 | CTDDVRPQGCYHGAGEQYRGTVSKTRKGVQCQRASAETPHKPQFTFTSEPHAQLEENFCR | 1288 | |
| | | \|\|\|\|\|\|\|\| \|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\| | | |
| Sbjct: | 361 | CTDDVRPQDCYHGAGEQYRGTVSKTRKGVQCQRWSAETPHKPQFTFTSEPHAQLEENFCR | 420 | |
| Query: | 1289 | DPDGDSYGPWCYTMDPRTPFDYCALRRCADDQPPSILDPPDQVQFEKCGKRVDRLDQRCS | 1468 | |
| | | +\|\|\|\|\|+\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \| | | |
| Sbjct: | 421 | NPDGDSHGPWCYTMDPRTPFDYCALRRCADDQPPSILDPPDQVQFEKCGKRVDRLDQRRS | 480 | |
| Query: | 1469 | KLRVAGGHPGNSPWTVSLRNRQGQHFCGGSLVKEQWILTARQCFSSSHMPLTGYEVWLGT | 1648 | |
| | | \|\|\|\| \|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| | | |
| Sbjct: | 481 | KLRVVGGHPGNSPWTVSLRNRQGQHFCGGSLVKEQWILTARQCFSSCHMPLTGYEVWLGT | 540 | |
| Query: | 1649 | LFQNPQHGEPGLQRVPVAKMLCGPSGSQLVLLKLERSVTLNQRVALICLPPEWYVVPPGT | 1828 | |
| | | \|\|\|\|\|\|\|\|\|\| \|\|\|\|\|\|\|\|\|+\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 541 | LFQNPQHGEPSLQRVPVAKMVCGPSGSQLVLLKLERSVTLNQRVALICLPPEWYVVPPGT | 600 | |
| Query: | 1829 | KCEIAGRGETKGTGNDTVLNVALLNVISNQECNIKHRGHVRESEMCTEGLLAPVGACEGG | 2008 | |
| | | \|\|\|\|\|\| \|\|\|\|\|\|\|\|\|\|\|\|\|\|\| \|\|\|\|\|\|\|\|\|\|\|\|\|\|\| \|\|\|\|\|\|\|\|\|\|\|\|\|\|\|\|\|\|\|\|\| | | |
| Sbjct: | 601 | KCEIAGWGETKGTGNDTVLNVAFLNVISNQECNIKHRGRVRESEMCTEGLLAPVGACEG- | 659 | |
| Query: | 2009 | DYGGPLACFTHNCWVLEGIRIPNRVCARSRWPAVFTRVSVFVDWIHKVMRLG 2164 | | |
| | | \|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\| | | |
| Sbjct: | 660 | DYGGPLACFTHNCWVLEGIIIPNRVCARSRWPAVFTRVSVFVDWIHKVMRLG 711 | | |

Other polypeptide sequences with homology to POLY13 are detailed in TABLE 14D.

TABLE 14D

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAR66602 Human L5/3 tumour suppressor protein - H . . . | +3 | 3875 | 0.0 | 1 |
| patp:AAW14270 Human growth factor L5/3 - Homo sapiens, . . . | +3 | 3875 | 0.0 | 1 |
| patp:AAY31157 Human macrophage stimulating protein - H . . . | +3 | 3871 | 0.0 | 1 |
| patp:AAW82789 Human NSF protein - Homo sapiens, 711 aa. +3 | 3864 | 0.0 | 1 | |
| patp:AAW07692 Macrophage stimulating protein C672X var . . . | +3 | 3853 | 0.0 | 1 |
| patp:AAR66603 Encoded by full-length human L5/3 tumour . . . | +3 | 3850 | 0.0 | 1 |
| patp:AAW14269 Human L5/3 reconstructed protein - Homo . . . | +3 | 3850 | 0.0 | 1 |
| patp:AAW07691 Macrophage stimulating protein C672 dele . . . | +3 | 3846 | 0.0 | 1 |
| patp:AAR66597 Human L5/3 tumour suppressor protein (Cy . . . | +3 | 3827 | 0.0 | 1 |
| patp:AAW14266 Human L5/3 partial clone #33 polymorphis . . . | +3 | 3827 | 0.0 | 1 |
| patp:AAR66598 Human L5/3 tumour suppressor protein (Ph . . . | +3 | 3816 | 0.0 | 1 |
| patp:AAW14267 Human L5/3 partial clone #33 polymorphis . . . | +3 | 3816 | 0.0 | 1 |
| patp:AAR66601 Mouse L5/3 tumour suppressor protein . . . | +3 | 3175 | 0.0 | 1 |
| patp:AAW14272 Mouse growth factor L5/3 complete protei . . . | +3 | 3175 | 0.0 | 1 |
| patp:AAY31156 Murine macrophage stimulating protein - . . . | +3 | 3175 | 0.0 | 1 |
| patp:AAW82790 Mouse MSP protein - Mus sp, 716 aa. | +3 | 3169 | 0.0 | 1 |
| patp:AAR66600 Mouse L5/3 tumour suppressor protein - M . . . | +3 | 3109 | 0.0 | 1 |
| patp:AAW14271 Mouse growth factor L5/3 partial cDNA cl . . . | +3 | 3109 | 0.0 | 1 |
| patp:AAR66599 Human L5/3 tumour suppressor protein (tr . . . | +3 | 2501 | 1.5e−261 | 2 |
| patp:AAW14268 Human L5/3 partial clone #19 protein - H . . . | +3 | 2501 | 1.5e−261 | 2 |
| patp:AAY06622 HGF-MSP hybrid protein alphabet-RTKR fac . . . | +3 | 2340 | 5.2e−242 | 1 |
| patp:AAY06621 HGF-MSP hybrid protein alphabet-1 factor . . . | +3 | 2339 | 6.7e−242 | 1 |
| patp:AAY06620 HGF-MSP hybrid protein Metron F-1 - Homo . . . | +3 | 1541 | 1.1e−197 | 2 |
| patp:AAR39521 Hepatocyte growth factor - Synthetic, 72 . . . | +3 | 1589 | 2.0e−162 | 1 |

PSORT analysis suggests that the protein may be localized in the lysosome with a certainty of 0.4202. Using SignalP analysis, it is predicted that the protein of the invention has a signal peptide with the most likely cleavage site between residues 18 and 19: VPG-QR (SEQ ID NO: 24). The predicted molecular weight is 80097.8 daltons.

Quantitative expression of POLY 13 was assessed as described in Example 4.

The family of macrophage-stimulating protein (MSP) precursors are also known as hepatocyte growth factor-like proteins (HGFL), and are structurally related to hepatocyte growth factor/scatter factor (HGF/SF). HGF/SF and MSP define a novel growth factor family whose members share the domain structure and the proteolytic process of activation of the blood proteinase precursor plasminogen. MSP and its tyrosine kinase receptor RON have been implicated in metastatic breast cancer. Therefore, poly13 and other members of the MSP family of proteins are useful in diagnostic and therapeutic applications implicated in disorders relating to cancer and metastatic potential.

POLY14: Tetracycline Transporter-Like Proteins and Nucleic Acids

Tetracyclines probably penetrate bacterial cells by passive diffusion and inhibit bacterial growth by interfering with protein synthesis or by destroying the membrane. A growing number of various bacterial species acquire resistance to the bacteriostatic activity of tetracycline. The two widespread mechanisms of bacterial resistance do not destroy tetracycline: one is mediated by efflux pumps, the other involves an EF-G-like protein that confers ribosome protection. Oxidative destruction of tetracycline has been found in a few species. Several efflux transporters, including multidrug-resistance pumps and tetracycline-specific exporters, confer bacterial resistance against tetracycline. Single amino acids of these carrier proteins important for tetracycline transport and substrate specificity have been identified, allowing the mechanism of tetracycline transport to begin to emerge. Resistance to tetracycline and other drugs is an important component in multidrug resistance, bacterial infections, cancer, and liver disease.

A novel nucleic acid was identified that is comprised of 1473 nucleotides (SEQ ID NO: 27), which encodes a tetracycline transporter like-like protein and is shown in Table 15A. An open reading frame was identified beginning with an ATG initiation codon at nucleotides 1-3 and ending with a TGA codon at nucleotides 1471-1473. The start and stop codons are in bold letters. Putative untranslated regions, if any, are found upstream from the initiation codon and downstream from the termination codon. The encoded protein having 490 amino acid residues (SEQ ID NO:28) is presented using the one-letter code in Table 15B.

TABLE 15A
The nucleotide sequence of POLY14.

| | |
|---|---|
| >3102960_EXT | (SEQ ID NO:27) |

ATGACCCAGGGGAAGAAGAAGAAACGGGCCGCGAACCGCAGTATCATGCTGGCCAAGAAGATCATCATTA
AGGACGGAGGCACGCCTCAAGGAATAGGTTCTCCTAGTGTCTATCATGCAGTTATCGTCATCTTTTTGGA
GTTTTTTGCTTGGGGACTATTGACAGCACCCACCTTGGTGGTATTACATGAAACCTTTCCTAAACATACA
TTTCTGATGAACGGCTTAATTCAAGGAGTAAAGGGTTTGTTGTCATTCCTTAGTGCCCCGCTTATTGGTG
CTCTTTCTGATGTTTGGGGCCGAAAATCCTTCTTGCTGCTAACGGTGTTTTTCACATGTGCCCCAATTCC
TTTAATGAAGATCAGCCCATGGTGGTACTTTGCTGTTATCTCTGTTTCTGGGGTTTTTGCAGTGACTTTT
TCTGTGGTATTTGCATACGTAGCAGATATAACCCAAGAGCATGAAAGAAGTATGGCTTATGGACTGGTAT
CAGCAACATTTGCTGCAAGTTTAGTCACCAGTCCTGCAATTGGAGCTTATCTTGGACGAGTATATGGGGA
CAGCTTGGTGGTGGTCTTAGCTACAGCAATAGCTTTGCTAGATATTTGTTTTATCCTTGTTGCTGTGCCA
GAGTCGTTGCCTGAGAAAATGCGGCCAGCATCCTGGGGAGCACCCATTTCCTGGGAACAAGCTGACCCTT
TTGCGTCCTTAAAAAAAGTCGGCCAAGATTCCATAGTGCTGCTGATCTGCATTACAGTGTTTCTCTCCTA
CCTACCGGAGGCAGGCCAATATTCCAGCTTTTTTTTATACCTCAGACAGATAATGAAATTTTCACCAGAA
AGTGTTGCAGCGTTTATAGCAGTCCTTGGCATTCTTTCCATTATTGCACAGACCATAGTCTTGAGTTTAC
TTATGAGGTCAATTGGAAATAAAAACACCATTTTACTGGGTCTAGGATTTCAAATATTACAGTTGGCATG
GTATGGCTTTGGTTCAAAACCTTGGATGATGTGGGCTGCTGGGGCAGTAGCAGCCATGTCTAGCATCACC
TTTCCTGCTGTCAGTGCACTTGTTTCACGAACTGCTGATGCTGATCAACAGGGTGTCGTTCAAGGAATGA
TAACAGGAATTCGAGGATTATGCAATGGTCTGGGACCGGCCCTCTATGGATTCATTTTCTACATATTCCA
TCTGGAACTTAAAGAACTGCCAATAACAGGAACAGACTTGGGAACAAACACAAGCCCTCAGCACCACTTT
GAACAGAATTCCATCATCCCTGGCCCTCCCTTCCTATTTGGAGCCTGTTCAGTACTGCTGGCTCTGCTTG
TTGCCTTGTTTATTCCGGAACATACCAATTTAAGCTTAAGGTCCAGCAGTTGGAGAAAGCACTGTGGCAG
TCACAGCCATCCTCATAATACACAAGCGCCAGGAGAGGCCAAAGAACCTTTACTCCAGGACACAAATGTG
TGA

TABLE 15B
Protein sequence encoded by the coding sequence shown in TABLE 15A

| | |
|---|---|
| >3102960_EXT | SEQ ID NO:28) |

MTQGKKKKRAANRSIMLAKKIIIKDGGTPQGIGSPSVYHAVIVIFLEFFAWGLLTAPTLVVLHETFPKHT
FLMNGLIQGVKGLLSFLSAPLIGALSDVWGRKSFLLLTVFFTCAPIPLMKISPWWYFAVISVSGVFAVTF
SVVFAYVADITQEHERSMAYGLVSATFAASLVTSPAIGAYLGRVYGDSLVVVLATAIALLDICFILVAVP
ESLPEKMRPASWGAPISWEQADPFASLKKVGQDSIVLLICITVFLSYLPEAGQYSSFFLYLRQIMKFSPE
SVAAFIAVLGILSIIAQTIVLSLLMRSIGNKNTILLGLGFQILQLAWYGFGSKPWMMWAAGAVAAMSSIT
FPAVSALVSRTADADQQGVVQGMITGIRGLCNGLGPALYGFIFYIFHVELKELPITGTDLGTNTSPQHHF
EQNSIIPGPPFLFGACSVLLALLVALFIPEHTNLSLRSSSWRKHCGSHSHPHNTQAPGEAKEPLLQDTNV

In a search of sequence databases, it was found, for example, that the nucleic acid sequence (SEQ ID NO:27) has 1353 of 1473 bases (91%) identical to a Mus musculus Tetracycline Transporter-like mRNA (GENBANK-ID: D88315). The full amino acid sequence of the protein of the invention was found to have 485 of 490 amino acid residues (98%) identical to, and 490 of 490 residues (100%) similar to, the 490 amino acid residue Tetracycline Transporter protein from Mus musculus (SPTREMBL-ACC:P70187)(Table 15C). The global sequence homology (as defined by GAP global sequence alignment with the full length sequence of this protein) is 60% amino acid similarity and 55% amino acid identity. In addition, this protein contains the following protein domains (as defined by Interpro) at the indicated amino acid positions: S-100 (IPR001751) at amino acid positions 20 to 60; and EF HAND (IPR002048) at amino acid positions 66 to 94.

TABLE 15C

BLASTX identity search against HIPPOCAMPUS ABUNDANT PROTEIN
TRANSCRIPT 1 (TETRACYCLINE TRANSPORTER-LIKE PROTEIN) - Mus
musculus (Mouse) (SEQ ID NO:43)

>ptnr:SPTREMBL-ACC:P70187 HIPPOCAMPUS ABUNDANT PROTEIN TRANSCRIPT 1
(TETRACYCLINE TRANSPORTER-LIKE PROTEIN) - Mus musculus (Mouse), 490 aa.
Plus Strand HSPs:
Score = 2494 (877.9 bits), Expect = 2.9e−258, P = 2.9e258
Identities = 485/490 (98%), Positives = 490/490 (100%), Frame = +1

| | | | | |
|---|---|---|---|---|
| Query: | 1 | MTQGKKKKRAANRSIMLAKKIIIKDGGTPQGIGSPSVYHAVIVIFLEFFAWGLLTAPTLV | 180 | (SEQ ID NO:28) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | MTQGKKKKRAANRSIMLAKKIIIKDGGTPQGIGSPSVYHAVIVIFLEFFAWGLLTAPTLV | 60 | (SEQ ID NO:43) |
| Query: | 181 | VLHETFPKHTFLMNGLIQGVKGLLSFLSAPLIGALSDVWGRKSFLLLTVFFTCAPIPLMK | 360 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | VLHETFPKHTFLMNGLIQGVKGLLSFLSAPLIGALSDVWGRKSFLLLTVFFTCAPIPLMK | 120 | |
| Query: | 361 | ISPWWYFAVISVSGVFAVTFSVVFAYVADITQEHERSMAYGLVSATFAASLVTSPAIGAY | 540 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | ISPWWYFAVISVSGVFAVTFSVVFAYVADITQEHERSMAYGLVSATFAASLVTSPAIGAY | 180 | |
| Query: | 541 | LGRVYGDSLVVVLATAIALLDICFILVAVPESLPEKMRPASWGAPISWEQADPFASLKKV | 720 | |
| | | \|\|++\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | LGQMYGDSLVVVLATAIALLDICFILVAVPESLPEKMRPASWGAPISWEQADPFASLKKV | 240 | |
| Query: | 721 | GQDSIVLLICITVFLSYLPEAGQYSSFFLYLRQIMKFSPESVAAFIAVLGILSIIAQTIV | 900 | |
| | | \|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\|\| | | |
| Sbjct: | 241 | GQDSIVLLICITVFLSYLPEAGQYSSFFLYLKQIMKFSPESVAAFIAVLGILSIIAQTIV | 300 | |
| Query: | 901 | LSLLMRSIGNKNTILLGLGFQILQLAWYGFGSKPWMMWAAGAVAAMSSITFPAVSALVSR | 1080 | |
| | | \|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\|\|\|\|\|\|\|\| | | |
| Sbjct: | 301 | LSLLMRSIGNKNTILLGLGFQILQLAWYGFGSEPWMMWAAGAVAAMSSITFPAVSALVSR | 360 | |
| Query: | 1081 | TADADQQGVVQGMITGIRGLCNGLGPALYGFIFYIFHVELKELPITGTDLGTNTSPQHHF | 1260 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | TADADQQGVVQGMITGIRGLCNGLGPALYGFIFYIFHVELKELPITGTDLGTNTSPQHHF | 420 | |
| Query: | 1261 | EQNSIIPGPPFLFGACSVLLALLVALFIPEHTNLSLRSSSWRKHCGSHSHPHNTQAPGEA | 1440 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\| | | |
| Sbjct: | 421 | EQNSIIPGPPFLFGACSVLLALLVALFIPEHTNLSLRSSSWRKHCGSHSHPHSTQAPGEA | 480 | |
| Query: | 1441 | KEPLLQDTNV 1470 | | |
| | | \|\|\|\|\|\|\|\|\|\| | | |
| Sbjct: | 481 | KEPLLQDTNV 490 | | |

Also, a POLY14 polypeptide has 433 of 438 (98%) residues identical and 438 of 438 (100%) residues similar to a human secreted protein sequence (PATP Accession No. AAB75294) as is shown in Table 15D. POLY14 homologies with other sequences are shown in Table 15E.

TABLE 15D

| | | | | |
|---|---|---|---|---|
| Query: | 157 | LLTAPTLVVLHETFPKHTFLMNGLIQGVKGLLSFLSAPLIGALSDVWGRKSFLLLTVFFT | 336 | (SEQ ID NO.28) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | LLTAPTLVVLHETFPKHTFLMNGLIQGVKGLLSFLSAPLIGALSDVWGRKSFLLLTVFFT | 60 | (SEQ ID NO.44) |
| Query: | 337 | CAPIPLMKISPWWYFAVISVSGVFAVTFSVVFAYVADITQEHERSMAYGLVSATFAASLV | 516 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 61 | CAPIPLMKISFWWYFAVISVSGVFAVTFSVVFAYVADITQEHERSMAYGLVSATFAASLV | 120 | |
| Query: | 517 | TSPAIGAYLGRVYGDSLVVVLATAIALLDICFILVAVPESLPEKMRPASWGAPISWEQAD | 696 | |
| | | \|\|\|\|\|\|\|\|\|\|++\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | TSPAIGAYLGQMYGDSLVVVLATAIALLDICFILVAVPESLPEKMRPASWGAPISWEQAD | 180 | |
| Query: | 697 | PFASLKKVGQDSIVLLICITVFLSYLPEAGQYSSFFLYLRQIMKFSPESVAAFIAVLGIL | 876 | |
| | | \|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\|\| | | |
| Sbjct: | 181 | PFASLKKVGQDSIVLLICITVFLSYLPEAGQYSSFFLYLKQIMKFSPESVAAFIAVLGIL | 240 | |
| Query: | 877 | SIIAQTIVLSLLMRSIGNKNTILLGLGFQILQLAWYGFGSKPWMMWAAGAVAAMSSITFP | 1056 | |
| | | \|\|\|\|\|\|\|\|\|\|\|\|\|\|\|\|\|\|\|\|\|\|\|\|\|\|\|\|\|\|\|\|\|\|\|\|\|\|\|\|+\|\|\|\|\|\|\|\|\|\|\|\|\|\|\|\|\|\|\| | | |
| Sbjct: | 241 | SIIAQTIVLSLLMRSIGNKNTILLGLGFQILQLAWYGFGSEPWMMWAAGAVAAMSSITFP | 300 | |
| Query: | 1057 | AVSALVSRTADADQQGVVQGMITGIRGLCNGLGPALYGFIFYIFHVELKELPITGTDLGT | 1236 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | AVSALVSRTADADQQGVVQGMITGIRGLCNGLGPALYGFIFYIFHVELKELPITGTDLGT | 360 | |
| Query: | 1237 | NTSPQHHFEQNSIIPGPPFLFGACSVLLALLVALFIPEHTNLSLRSSSWRKHCGSHSHPH | 1416 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | NTSPQNHFEQNSIIPGPPFLFGACSVLLALLVALFIPEHTNLSLRSSSWRKHCGSHSHPH 420 | | |
| Query: | 1417 | NTQAPGEAKEPLLQDTNV 1470 | | |
| | | +\|\|\|\|\|\|\|\|\|\|\|\|\|\|\|\|\| | | |
| Sbjct: | 421 | STQAPGEAKEPLLQDTNV 438 | | |

TABLE 15E

| | | |
|---|---|---|
| | | Smallest |
| | | Sum |
| Reading | High | Probability |

| | | | | |
|---|---|---|---|---|
| Sequences producing High-scoring Segment Pairs: | Frame | Score | P(N) | N |

| | | | | |
|---|---|---|---|---|
| patp:AAB75294 Gene 7 human secreted protein homologous . . . | +1 | 2228 | 3.8e−230 | 1 |
| patp:AAB58289 Lung cancer associated polypeptide seque . . . | +1 | 1795 | 2.9e−184 | 1 |
| patp:AAB75295 Human secreted protein sequence encoded . . . | +1 | 1689 | 5.0e−173 | 1 |
| patp:AAY29332 Human secreted protein clone pe584_2 pro . . . | +1 | 1670 | 5.2e−171 | 1 |
| patp:AAB75246 Human secreted protein sequence encoded . . . | +1 | 1396 | 5.6e−142 | 1 |
| patp:AAW74870 Human secreted protein encoded by gene 1 . . . | +1 | 983 | 3.3e−98 | 1 |

PSORT analysis suggests that the protein may be localized in the plasma membrane with a certainty of 0.600. Using SignalP analysis, it is predicted that the protein of the invention has a signal peptide with the most likely cleavage site between residues 55 and 56:: LLT-AP (SEQ ID NO: 28). The predicted molecular weight is 53025.7 daltons.

Quantitative expression of POLY14 was assessed as described in Example 4.

The tetracycline transporter protein family is conserved from bacteria to humans, and are important in multidrug resistance. Therefore, new members of the tetracycline transporter protein family are useful in diagnostic and therapeutic applications implicated in disorders relating to multidrug resistance important in bacterial infections, cancer and liver disease.

POLYX Nucleic Acids

The novel nucleic acids of the invention include those that encode a POLYX or POLYX-like protein, or biologically-active portions thereof. The nucleic acids include nucleic acids encoding polypeptides that include the amino acid sequence of one or more of SEQ ID NO:2n (wherein n=1 to 14). The encoded polypeptides can thus include, e.g., the amino acid sequences of SEQ ID NO:2, 4, 6, 8, 10, 12, 14, 16, 18, 20, 22, 24, 26 and/or 28.

In some embodiments, a nucleic acid encoding a polypeptide having the amino acid sequence of one or more of SEQ ID NO:2n (wherein n=1 to 14) includes the nucleic acid sequence of any of SEQ ID NO:2n-1 (wherein n=1 to 14), or a fragment thereof, and can thus include, e.g., the nucleic acid sequences of SEQ ID NO: 1, 3, 5, 7, 9, 11, 13, 15, 17, 19, 21, 23, 25 and/or 27. Additionally, the invention includes mutant or variant nucleic acids of any of SEQ ID NO:2n-1 (wherein n=1 to 14), or a fragment thereof, any of whose bases may be changed from the disclosed sequence while still encoding a protein that maintains its POLYX-like biological activities and physiological functions. The invention further includes the complement of the nucleic acid sequence of any of SEQ ID NO:2n-1 (wherein n=1 to 14), including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify POLYX-encoding nucleic acids (e.g., POLYX mRNA) and fragments for use as polymerase chain reaction (PCR) primers for the amplification or mutation of POLYX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

As utilized herein, the term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

As utilized herein, the term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated POLYX nucleic acid molecule can contain less than approximately 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

A nucleic acid molecule of the invention, e.g., a nucleic acid molecule having the nucleotide sequence of SEQ ID NO:2n-1 (wherein n=1 to 14), or a complement of any of these nucleotide sequences, can be isolated using standard molecular biology techniques and the sequence information provided herein. Using all or a portion of the nucleic acid sequence of any of SEQ ID NO:2n-1 (wherein n=1 to 14) as a hybridization probe, POLYX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to POLYX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NO:2n-1 (wherein n=1 to 14), or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in any of SEQ ID NO:2n-1 (wherein n=1 to 14). In still another embodiment, an isolated nucleic acid molecule of the invention comprises a nucleic acid molecule that is a complement of the nucleotide sequence shown in any of SEQ ID NO:2n-1 (wherein n=1 to 14), or a portion of this nucleotide sequence. A nucleic acid molecule that is complementary to the nucleotide sequence shown in any of SEQ ID NO:2n-1 (wherein n=1 to 14) is one that is sufficiently complementary to the nucleotide sequence shown that it can hydrogen bond with little or no mismatches to the nucleotide sequence shown in of any of SEQ ID NO:2n-1 (wherein n=1 to 14), thereby forming a stable duplex.

As used herein, the term “complementary” refers to Watson-Crick or Hoogsteen base-pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of SEQ ID NO:2n-1 (wherein n=1 to 14), e.g., a fragment that can be used as a probe or primer, or a fragment encoding a biologically active portion of POLYX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described infra. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

As utilized herein, the term “homologous nucleic acid sequence” or “homologous amino acid sequence,” or variations thereof, refer to sequences characterized by a homology at the nucleotide level or amino acid level as discussed supra. Homologous nucleotide sequences encode those sequences coding for isoforms of POLYX polypeptide. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a POLYX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human POLYX protein. Homologous nucleic acid sequences include those nucleic acid sequences that encode conservative amino acid substitutions (see below) in any of SEQ ID NO:2n (wherein n=1 to 14) as well as a polypeptide having POLYX activity. Biological activities of the POLYX proteins are described below. A homologous amino acid sequence does not encode the amino acid sequence of a human POLYX polypeptide.

The nucleotide sequence determined from the cloning of the human POLYX gene allows for the generation of probes and primers designed for use in identifying the cell types disclosed and/or cloning POLYX homologues in other cell types, e.g., from other tissues, as well as POLYX homologues from other mammals. The probe/primer typically comprises a substantially-purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:2n-1 (wherein n=1 to 14); or an anti-sense strand nucleotide sequence of SEQ ID NO:2n-1 (wherein n=1 to 14); or of a naturally occurring mutant of SEQ ID NO:2n-1 (wherein n=1 to 14).

Probes based upon the human POLYX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which mis-express a POLYX protein, such as by measuring a level of a POLYX-encoding nucleic acid in a sample of cells from a subject e.g., detecting POLYX mRNA levels or determining whether a genomic POLYX gene has been mutated or deleted.

As utilized herein, the term “a polypeptide having a biologically-active portion of POLYX refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of POLYX can be prepared by isolating a portion of SEQ ID NO:2n-1 (wherein n=1 to 14), that encodes a polypeptide having a POLYX biological activity, expressing the encoded portion of POLYX protein (e.g., by recombinant expression in vitro), and assessing the activity of the encoded portion of POLY.

POLYX Variants

The invention further encompasses nucleic acid molecules that differ from the disclosed POLYX nucleotide sequences due to degeneracy of the genetic code. These nucleic acids therefore encode the same POLYX protein as those encoded by the nucleotide sequence shown in SEQ ID NO:2n-1 (wherein n=1 to 14). In another embodiment, an isolated nucleic acid molecule of the invention has a nucleotide sequence encoding a protein having an amino acid sequence shown in any of SEQ ID NO:2n (wherein n =1 to 14).

In addition to the human POLYX nucleotide sequence shown in any of SEQ ID NO:2n-1 (wherein n=1 to 14), it will be appreciated by those skilled in the art that DNA sequence polymorphisms that lead to changes in the amino acid sequences of POLYX may exist within a population (e.g., the human population). Such genetic polymorphism in the POLYX gene may exist among individuals within a population due to natural allelic variation. As used herein, the terms “gene” and “recombinant gene” refer to nucleic acid molecules comprising an open reading frame encoding a POLYX protein, preferably a mammalian POLYX protein. Such natural allelic variations can typically result in 1-5% variance in the nucleotide sequence of the POLYX gene. Any and all such nucleotide variations and resulting amino acid polymorphisms in POLYX that are the result of natural allelic variation and that do not alter the functional activity of POLYX are intended to be within the scope of the invention.

Additionally, nucleic acid molecules encoding POLYX proteins from other species, and thus that have a nucleotide sequence that differs from the human sequence of any of SEQ ID NO:2n-1 (wherein n=1 to 14), are intended to be within the scope of the invention. Nucleic acid molecules corresponding to natural allelic variants and homologues of the POLYX cDNAs of the invention can be isolated based on their homology to the human POLYX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NO:2n-1 (wherein n=1 to 14). In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding POLYX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2× SSC, 0.01% BSA at 50° C. An isolated nucleic acid molecule of the invention that hybridizes under stringent conditions to the sequence of any of SEQ ID NO:2n-1 (wherein n=1 to 14) corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of any of SEQ ID NO:2n-1 (wherein n=1 to 14), or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990. GENE TRANSFER AND ExPRESSION, A LABORATORY MANUAL, Stockton Press, NY.

In a third embodiment, a nucleic acid that is hybridizable to the nucleic acid molecule comprising the nucleotide sequence of any of SEQ ID NO:2n-1 (wherein n=1 to 14), or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCI (pH 7.4), 5 mM EDTA, and 0.1% SDS at 50° C. Other conditions of low stringency that may be used are well known in the art (e.g., as employed for cross-species hybridizations). See, e.g., Ausubel, et al., (eds.), 1993. CURRENT PROTOCOLS IN MOLECULAR BIOLOGY, John Wiley & Sons, NY, and Kriegler, 1990. GENE TRANSFER AND EXPRESSION, A LABORATORY MANUAL, Stockton Press, NY; Shilo and Weinberg, 1981. Proc. Natl. Acad. Sci. USA 78: 6789-6792.

Conservative Mutations

In addition to naturally-occurring allelic variants of the POLYX sequence that may exist in the population, the skilled artisan will further appreciate that changes can be introduced by mutation into the nucleotide sequence of any of SEQ ID NO:2n-1 (wherein n=1 to 14), thereby leading to changes in the amino acid sequence of the encoded POLYX protein, without altering the functional ability of the POLYX protein. For example, nucleotide substitutions leading to amino acid substitutions at “non-essential” amino acid residues can be made in the sequence of any of SEQ ID NO:2n-1 (wherein n=1 to 14). A “non-essential” amino acid residue is a residue that can be altered from the wild-type sequence of POLYX without altering the biological activity, whereas an “essential” amino acid residue is required for biological activity. For example, amino acid residues that are conserved among the POLYX proteins of the invention, are predicted to be particularly non-amenable to such alteration.

Amino acid residues that are conserved among members of a POLYX family are predicted to be less amenable to alteration. For example, a POLYX protein according to the invention can contain at least one domain that is a typically conserved region in a POLYX family member. As such, these conserved domains are not likely to be amenable to mutation. Other amino acid residues, however, (e.g., those that are not conserved or only semi-conserved among members of the POLYX family) may not be as essential for activity and thus are more likely to be amenable to alteration.

Another aspect of the invention pertains to nucleic acid molecules encoding POLYX proteins that contain changes in amino acid residues that are not essential for activity. Such POLYX proteins differ in amino acid sequence from any of any of SEQ ID NO:2n (wherein n=1 to 14), yet retain biological activity. In one embodiment, the isolated nucleic acid molecule comprises a nucleotide sequence encoding a protein, wherein the protein comprises an amino acid sequence at least about 75% homologous to the amino acid sequence of any of SEQ ID NO:2n (wherein n=1 to 14). Preferably, the protein encoded by the nucleic acid is at least about 80% homologous to any of SEQ ID NO:2n (wherein n=1 to 14), more preferably at least about 90%, 95%, 98%, and most preferably at least about 99% homologous to SEQ ID NO:2n (wherein n=1 to 14).

An isolated nucleic acid molecule encoding a POLYX protein homologous to the protein of any of SEQ ID NO:2n (wherein n=1 to 14) can be created by introducing one or more nucleotide substitutions, additions or deletions into the corresponding nucleotide sequence (i.e., SEQ ID NO:2n-1 for the corresponding n), such that one or more amino acid substitutions, additions or deletions are introduced into the encoded protein.

Mutations can be introduced into SEQ ID NO:2n-1 (wherein n =1 to 14) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in POLYX is replaced with another amino acid residue from the same side chain family. Alternatively, in another embodiment, mutations can be introduced randomly along all or part of a POLYX coding sequence, such as by saturation mutagenesis, and the resultant mutants can be screened for POLYX biological activity to identify mutants that retain activity. Following mutagenesis of SEQ ID NO:2n-1 (wherein n=1 to 14the encoded protein can be expressed by any recombinant technology known in the art and the activity of the protein can be determined.

In one embodiment, a mutant POLYX protein can be assayed for: (i) the ability to form protein:protein interactions with other POLYX proteins, other cell-surface proteins, or biologically-active portions thereof; (ii) complex formation between a mutant POLYX protein and a POLYX receptor; (iii) the ability of a mutant POLYX protein to bind to an intracellular target protein or biologically active portion thereof; (e.g., avidin proteins); (iv) the ability to bind BRA protein; or (v) the ability to specifically bind an anti-POLYX protein antibody.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1 (wherein n=1 to 14), or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire POLYX coding strand, or to only a portion thereof. Nucleic acid molecules encoding fragments, homologs, derivatives and analogs of a POLYX protein of any of SEQ ID NO:2n (wherein n=1 to 14) or antisense nucleic acids complementary to a POLYX nucleic acid sequence of SEQ ID NO:2n-1 (wherein n=1 to 14) are additionally provided.

In one embodiment, an antisense nucleic acid molecule is antisense to a “coding region” of the coding strand of a nucleotide sequence encoding POLY. The term “coding region” refers to the region of the nucleotide sequence comprising codons which are translated into amino acid residues (e.g., the protein coding region of a human POLYX that corresponds to any of SEQ ID NO:2n (wherein n=1 to 14)). In another embodiment, the antisense nucleic acid molecule is antisense to a “non-coding region” of the coding strand of a nucleotide sequence encoding POLY. The term “non-coding region” refers to 5′ and 3′ sequences which flank the coding region that are not translated into amino acids (i.e., also referred to as 5′ and 3′ non-translated regions).

Given the coding strand sequences encoding POLYX disclosed herein (e.g., SEQ ID NO:2n-1 (wherein n=1 to 14)), antisense nucleic acids of the invention can be designed according to the rules of Watson and Crick or Hoogsteen base-pairing. The antisense nucleic acid molecule can be complementary to the entire coding region of POLYX mRNA, but more preferably is an oligonucleotide that is antisense to only a portion of the coding or non-coding region of POLYX mRNA. For example, the antisense oligonucleotide can be complementary to the region surrounding the translation start site of POLYX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a POLYX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave POLYX mRNA transcripts to thereby inhibit translation of POLYX mRNA. A ribozyme having specificity for a POLYX-encoding nucleic acid can be designed based upon the nucleotide sequence of a POLYX DNA disclosed herein (i.e., SEQ ID NO:2n-1 (wherein n=1 to 14)). For example, a derivative of a Tetrahymena L-19 IVS RNA can be constructed in which the nucleotide sequence of the active site is complementary to the nucleotide sequence to be cleaved in a POLYX-encoding MRNA. See, e.g., Cech, et al., U.S. Pat. No. 4,987,071; and Cech, et al., U.S. Pat. No. 5,116,742. Alternatively, POLYX mRNA can be used to select a catalytic RNA having a specific ribonuclease activity from a pool of RNA molecules (Bartel, et al., 1993. Science 261: 1411-1418).

Alternatively, POLYX gene expression can be inhibited by targeting nucleotide sequences complementary to the regulatory region of the POLYX (e.g., the POLYX promoter and/or enhancers) to form triple helical structures that prevent transcription of the POLYX gene in target cells. See, e.g., Helene, 1991. Anticancer Drug Des. 6: 569-84; Helene, et al., 1992. Ann. N.Y. Acad. Sci. 660: 27-36; and Maher, 1992. Bioassays 14: 807-15.

In various embodiments, the nucleic acids of POLYX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg. Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of POLYX can be used in therapeutic and diagnostic applications. For example, PNAs can be used as antisense or antigene agents for sequence-specific modulation of gene expression by, e.g., inducing transcription or translation arrest or inhibiting replication. PNAs of POLYX can also be used, e.g., in the analysis of single base pair mutations in a gene by, e.g., PNA directed PCR clamping; as artificial restriction enzymes when used in combination with other enzymes, e.g., S1 nucleases (see, Hyrup, 1996., supra); or as probes or primers for DNA sequence and hybridization (see, Hyrup, et al., 1996.; Perry-O'Keefe, 1996., supra).

In another embodiment, PNAs of POLYX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of POLYX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., supra). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., supra). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like. Characterization of POLYX Polypeptides A polypeptide according to the invention includes a polypeptide including the amino acid sequence of POLYX polypeptides whose sequences are provided in any SEQ ID NO:2n (wherein n=1 to 14) and includes SEQ ID NOS:2, 4, 6, 8, 10, 12, 14, 16, 18, 20, 22, 24, 26 and/or 28. The invention also includes a mutant or variant protein any of whose residues may be changed from the corresponding residues shown in SEQ ID NOS: 2, 4, 6, 8, 10, 12, 14, 16, 18, 20, 22, 24, 26 and/or 28, while still encoding a protein that maintains its POLYX activities and physiological functions, or a functional fragment thereof.

In general, a POLYX variant that preserves POLYX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated POLYX proteins, and biologically-active portions thereof, or derivatives, fragments, analogs or homologs thereof. Also provided are polypeptide fragments suitable for use as immunogens to raise anti-POLYX antibodies. In one embodiment, native POLYX proteins can be isolated from cells or tissue sources by an appropriate purification scheme using standard protein purification techniques. In another embodiment, POLYX proteins are produced by recombinant DNA techniques. Alternative to recombinant expression, a POLYX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the POLYX protein is derived, or substantially free from chemical precursors or other chemicals when chemically synthesized. The language “substantially free of cellular material” includes preparations of POLYX proteins in which the protein is separated from cellular components of the cells from which it is isolated or recombinantly-produced. In one embodiment, the language “substantially free of cellular material” includes preparations of POLYX proteins having less than about 30% (by dry weight) of non-POLYX proteins (also referred to herein as a “contaminating protein”), more preferably less than about 20% of non-POLYX proteins, still more preferably less than about 10% of non-POLYX proteins, and most preferably less than about 5% of non-POLYX proteins. When the POLYX protein or biologically-active portion thereof is recombinantly-produced, it is also preferably substantially free of culture medium, i.e., culture medium represents less than about 20%, more preferably less than about 10%, and most preferably less than about 5% of the volume of the POLYX protein preparation.

As utilized herein, the phrase “substantially free of chemical precursors or other chemicals” includes preparations of POLYX protein in which the protein is separated from chemical precursors or other chemicals that are involved in the synthesis of the protein. In one embodiment, the language “substantially free of chemical precursors or other chemicals” includes preparations of POLYX protein having less than about 30% (by dry weight) of chemical precursors or non-POLYX chemicals, more preferably less than about 20% chemical precursors or non-POLYX chemicals, still more preferably less than about 10% chemical precursors or non-POLYX chemicals, and most preferably less than about 5% chemical precursors or non-POLYX chemicals.

Biologically-active portions of a POLYX protein include peptides comprising amino acid sequences sufficiently homologous to or derived from the amino acid sequence of the POLYX protein which include fewer amino acids than the full-length POLYX proteins, and exhibit at least one activity of a POLYX protein. Typically, biologically-active portions comprise a domain or motif with at least one activity of the POLYX protein. A biologically-active portion of a POLYX protein can be a polypeptide which is, for example, 10, 25, 50, 100 or more amino acids in length.

A biologically-active portion of a POLYX protein of the invention may contain at least one of the above-identified conserved domains. Moreover, other biologically active portions, in which other regions of the protein are deleted, can be prepared by recombinant techniques and evaluated for one or more of the functional activities of a native POLYX protein.

In an embodiment, the POLYX protein has an amino acid sequence shown in any of SEQ ID NO:2n (wherein n=1 to 14). In other embodiments, the POLYX protein is substantially homologous to any of SEQ ID NO:2n (wherein n=1 to 14) and retains the functional activity of the protein of any of SEQ ID NO:2n (wherein n=1 to 14), yet differs in amino acid sequence due to natural allelic variation or mutagenesis, as described in detail below. Accordingly, in another embodiment, the POLYX protein is a protein that comprises an amino acid sequence at least about 45% homologous, and more preferably about 55, 65, 70, 75, 80, 85, 90, 95, 98 or even 99% homologous to the amino acid sequence of any of SEQ ID NO:2n (wherein n=1 to 14) and retains the functional activity of the POLYX proteins of the corresponding polypeptide having the sequence of SEQ ID NO:2n (wherein n=1 to 14).

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:2n-1 (wherein n=1 to 14), e.g., SEQ ID NOS:1, 3, 5, 7, 9, 11, 13, 15, 17, 19, 21, 23, 25 and/or 27.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides POLYX chimeric or fusion proteins. As used herein, a POLYX “chimeric protein” or “fusion protein” comprises a POLYX polypeptide operatively-linked to a non-POLYX polypeptide. An “POLYX polypeptide” refers to a polypeptide having an amino acid sequence corresponding to a POLYX protein shown in SEQ ID NO:2n (wherein n=1 to 14), [e.g., SEQ ID NO:2, 4, 6, 8, 10, 12, 14, 16, 18, 20, 22, 24,26 and/or 28], whereas a “non-POLYX polypeptide” refers to a polypeptide having an amino acid sequence corresponding to a protein that is not substantially homologous to the POLYX protein (e.g., a protein that is different from the POLYX protein and that is derived from the same or a different organism). Within a POLYX fusion protein the POLYX polypeptide can correspond to all or a portion of a POLYX protein. In one embodiment, a POLYX fusion protein comprises at least one biologically-active portion of a POLYX protein. In another embodiment, a POLYX fusion protein comprises at least two biologically-active portions of a POLYX protein. In yet another embodiment, a POLYX fusion protein comprises at least three biologically-active portions of a POLYX protein. Within the fusion protein, the term “operatively-linked” is intended to indicate that the POLYX polypeptide and the non-POLYX polypeptide are fused in-frame with one another. The non-POLYX polypeptide can be fused to the amino-terminus or carboxyl-terminus of the POLYX polypeptide.

In one embodiment, the fusion protein is a GST-POLYX fusion protein in which the POLYX sequences are fused to the carboxyl-terminus of the GST (glutathione S-transferase) sequences. Such fusion proteins can facilitate the purification of recombinant POLYX polypeptides.

In another embodiment, the fusion protein is a POLYX protein containing a heterologous signal sequence at its amino-terminus. In certain host cells (e.g., mammalian host cells), expression and/or secretion of POLYX can be increased through use of a heterologous signal sequence.

In yet another embodiment, the fusion protein is a POLYX-immunoglobulin fusion protein in which the POLYX sequences are fused to sequences derived from a member of the immunoglobulin protein family. The POLYX-immunoglobulin fusion proteins of the invention can be incorporated into pharmaceutical compositions and administered to a subject to inhibit an interaction between a POLYX ligand and a POLYX protein on the surface of a cell, to thereby suppress POLYX-mediated signal transduction in vivo. The POLYX-immunoglobulin fusion proteins can be used to affect the bioavailability of a POLYX cognate ligand. Inhibition of the POLYX ligand/POLYX interaction may be useful therapeutically for both the treatment of proliferative and differentiative disorders, as well as modulating (e.g., promoting or inhibiting) cell survival. Moreover, the POLYX-immunoglobulin fusion proteins of the invention can be used as immunogens to produce anti-POLYX antibodies in a subject, to purify POLYX ligands, and in screening assays to identify molecules that inhibit the interaction of POLYX with a POLYX ligand.

A POLYX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A POLYX-encoding nucleic acid can be cloned into such an expression vector such that the fusion moiety is linked in-frame to the POLYX protein.

POLYX Agonists and Antagonists

The invention also pertains to variants of the POLYX proteins that function as either POLYX agonists (i.e., mimetics) or as POLYX antagonists. Variants of the POLYX protein can be generated by mutagenesis (e.g., discrete point mutation or truncation of the POLYX protein). An agonist of a POLYX protein can retain substantially the same, or a subset of, the biological activities of the naturally-occurring form of a POLYX protein. An antagonist of a POLYX protein can inhibit one or more of the activities of the naturally occurring form of a POLYX protein by, for example, competitively binding to a downstream or upstream member of a cellular signaling cascade which includes the POLYX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the POLYX proteins.

Variants of the POLYX proteins that function as either POLYX agonists (i.e., mimetics) or as POLYX antagonists can be identified by screening combinatorial libraries of mutants (e.g., truncation mutants) of the POLYX proteins for POLYX protein agonist or antagonist activity. In one embodiment, a variegated library of POLYX variants is generated by combinatorial mutagenesis at the nucleic acid level and is encoded by a variegated gene library. A variegated library of POLYX variants can be produced by, for example, enzymatically-ligating a mixture of synthetic oligonucleotides into gene sequences such that a degenerate set of potential POLYX sequences is expressible as individual polypeptides, or alternatively, as a set of larger fusion proteins (e.g., for phage display) containing the set of POLYX sequences therein. There are a variety of methods which can be used to produce libraries of potential POLYX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential POLYX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the POLYX protein coding sequences can be used to generate a variegated population of POLYX fragments for screening and subsequent selection of variants of a POLYX protein. In one embodiment, a library of coding sequence fragments can be generated by treating a double-stranded PCR fragment of a POLYX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes amino-terminal and internal fragments of various sizes of the POLYX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of POLYX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify POLYX variants. See, e.g., Arkin and Yourvan, 1992. Proc. Natl. Acad. Sc. USA 89: 7811-7815; Delgrave, et al., 1993. Protein Engineering 6:327-331.

Anti-POLYX Antibodies

The invention encompasses antibodies and antibody fragments, such as F_{ab} or (F_{ab})₂, that bind immunospecifically to any of the POLYX polypeptides of said invention.

An isolated POLYX protein, or a portion or fragment thereof, can be used as an immunogen to generate antibodies that bind to POLYX polypeptides using standard techniques for polyclonal and monoclonal antibody preparation. The full-length POLYX proteins can be used or, alternatively, the invention provides antigenic peptide fragments of POLYX proteins for use as immunogens. The antigenic POLYX peptides comprises at least 4 amino acid residues of the amino acid sequence shown in SEQ ID NO:2n (wherein n=1 to 14) and encompasses an epitope of POLYX such that an antibody raised against the peptide forms a specific immune complex with POLY. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of POLYX that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, POLYX protein sequences of SEQ ID NO:2n (wherein n=1 to 14), or derivatives, fragments, analogs, or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as POLY. Such antibodies include, but are not limited to, polyclonal, monoclonal, chimeric, single chain, F_{ab} and F_{(ab′})₂ fragments, and an F_{ab} expression library. In a specific embodiment, antibodies to human POLYX proteins are disclosed. Various procedures known within the art may be used for the production of polyclonal or monoclonal antibodies to a POLYX protein sequence of SEQ ID NO:2n (wherein n=1 to 14), or a derivative, fragment, analog, or homolog thereof. Some of these proteins are discussed, infra.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed POLYX protein or a chemically-synthesized POLYX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against POLYX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of POLY. A monoclonal antibody composition thus typically displays a single binding affinity for a particular POLYX protein with which it immunoreacts. For preparation of monoclonal antibodies directed towards a particular POLYX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc. Natl. Acad. Sci. USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to a POLYX protein (see, e.g., U.S. Pat. No. 4,946,778). In addition, methods can be adapted for the construction of Fab expression libraries (see, e.g., Huse, et al., 1989. Science 246: 1275-1281) to allow rapid and effective identification of monoclonal Fab fragments with the desired specificity for a POLYX protein or derivatives, fragments, analogs or homologs thereof. Non-human antibodies can be “humanized” by techniques well known in the art. See, e.g., U.S. Pat. No. 5,225,539. Antibody fragments that contain the idiotypes to a POLYX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab)2} fragment; (iii) an F_{ab} fragment generated by the treatment of the antibody molecule with papain and a reducing agent and (iv) Fᵥ fragments.

Additionally, recombinant anti-POLYX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., 1988. Science 240: 1041-1043; Liu, et al., 1987. Proc. Natl. Acad. Sci USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison (1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a POLYX protein is facilitated by generation of hybridomas that bind to the fragment of a POLYX protein possessing such a domain. Thus, antibodies that are specific for a desired domain within a POLYX protein, or derivatives, fragments, analogs or homologs thereof, are also provided herein.

Anti-POLYX antibodies may be used in methods known within the art relating to the localization and/or quantitation of a POLYX protein (e.g., for use in measuring levels of the POLYX protein within appropriate physiological samples, for use in diagnostic methods, for use in imaging the protein, and the like). In a given embodiment, antibodies for POLYX proteins, or derivatives, fragments, analogs or homologs thereof, that contain the antibody derived binding domain, are utilized as pharmacologically-active compounds (hereinafter “Therapeutics”).

An anti-POLYX antibody (e.g., monoclonal antibody) can be used to isolate a POLYX polypeptide by standard techniques, such as affinity chromatography or immunoprecipitation. An anti-POLYX antibody can facilitate the purification of natural POLYX polypeptide from cells and of recombinantly-produced POLYX polypeptide expressed in host cells. Moreover, an anti-POLYX antibody can be used to detect POLYX protein (e.g., in a cellular lysate or cell supernatant) in order to evaluate the abundance and pattern of expression of the POLYX protein. Anti-POLYX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

POLYX Recombinant Expression Vectors and Host Cells

Another aspect of the invention pertains to vectors, preferably expression vectors, containing a nucleic acid encoding a POLYX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present Specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

As utilized herein, the phrase “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., POLYX proteins, mutant forms of POLYX proteins, fusion proteins, etc.).

The recombinant expression vectors of the invention can be designed for expression of POLYX proteins in prokaryotic or eukaryotic cells. For example, POLYX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., GENE EXPRESSION TECHNOLOGY: METHODS INENZYMOLOGY 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, GENE ExPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al., 1992. Nuc. Acids Res. 20: 2111-2118). Such alteration of nucleic acid sequences of the invention can be carried out by standard DNA synthesis techniques.

In another embodiment, the POLYX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, POLYX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; see, Pinkert, et al., 1987. Genes Dev. 1: 268-277), lymphoid-specific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Banerji, et al., 1983. Cell 33: 729-740; Queen and Baltimore, 1983. Cell 33: 741-748), neuron-specific promoters (e.g., the neurofilament promoter; see, Byrne and Ruddle, 1989. Proc. Natl. Acad. Sci. USA 86: 5473-5477), pancreas-specific promoters (see, Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to POLYX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, POLYX protein can be expressed in bacterial cells such as Escherichia coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, and methotrexate. Nucleic acid encoding a selectable marker can be introduced into a host cell on the same vector as that encoding POLYX or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) POLYX protein. Accordingly, the invention further provides methods for producing POLYX protein using the host cells of the invention. In one embodiment, the method comprises culturing the host cell of invention (i.e., into which a recombinant expression vector encoding POLYX protein has been introduced) in a suitable medium such that POLYX protein is produced. In another embodiment, the method further comprises isolating POLYX protein from the medium or the host cell.

Transgenic Animals

The host cells of the invention can also be used to produce non-human transgenic animals. For example, in one embodiment, a host cell of the invention is a fertilized oocyte or an embryonic stem cell into which POLYX protein-coding sequences have been introduced. These host cells can then be used to create non-human transgenic animals in which exogenous POLYX sequences have been introduced into their genome or homologous recombinant animals in which endogenous POLYX sequences have been altered. Such animals are useful for studying the function and/or activity of POLYX protein and for identifying and/or evaluating modulators of POLYX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous POLYX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing POLYX-encoding nucleic acid into the male pronuclei of a fertilized oocyte (e.g., by micro-injection, retroviral infection) and allowing the oocyte to develop in a pseudopregnant female foster animal. The human POLYX cDNA sequences of SEQ ID NO:2n-1 (wherein n=1 to 14), can be introduced as a transgene into the genome of a non-human animal. Alternatively, a non-human homologue of the human POLYX gene, such as a mouse POLYX gene, can be isolated based on hybridization to the human POLYX cDNA (described further supra) and used as a transgene. Intronic sequences and polyadenylation signals can also be included in the transgene to increase the efficiency of expression of the transgene. A tissue-specific regulatory sequence(s) can be operably-linked to the POLYX transgene to direct expression of POLYX protein to particular cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the POLYX transgene in its genome and/or expression of POLYX mRNA in tissues or cells of the animals. A transgenic founder animal can then be used to breed additional animals carrying the transgene. Moreover, transgenic animals carrying a transgene-encoding POLYX protein can further be bred to other transgenic animals carrying other transgenes.

To create a homologous recombinant animal, a vector is prepared which contains at least a portion of a POLYX gene into which a deletion, addition or substitution has been introduced to thereby alter, e.g., functionally disrupt, the POLYX gene. The POLYX gene can be a human gene (e.g., the cDNA of SEQ ID NO:2n-1 (wherein n=1 to 14)), but more preferably, is a non-human homologue of a human POLYX gene. For example, a mouse homologue of human POLYX gene of SEQ ID NO:2n-l (wherein n=1 to 14), can be used to construct a homologous recombination vector suitable for altering an endogenous POLYX gene in the mouse genome. In one embodiment, the vector is designed such that, upon homologous recombination, the endogenous POLYX gene is functionally disrupted (i.e., no longer encodes a functional protein; also referred to as a “knock out” vector).

Alternatively, the vector can be designed such that, upon homologous recombination, the endogenous POLYX gene is mutated or otherwise altered but still encodes functional protein (e.g., the upstream regulatory region can be altered to thereby alter the expression of the endogenous POLYX protein). In the homologous recombination vector, the altered portion of the POLYX gene is flanked at its 5′- and 3′-termini by additional nucleic acid of the POLYX gene to allow for homologous recombination to occur between the exogenous POLYX gene carried by the vector and an endogenous POLYX gene in an embryonic stem cell. The additional flanking POLYX nucleic acid is of sufficient length for successful homologous recombination with the endogenous gene. Typically, several kilobases (Kb) of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced POLYX gene has homologously-recombined with the endogenous POLYX gene are selected. See, e.g., Li, et al., 1992. Cell 69: 915.

The selected cells are then micro-injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte, and then transferred to pseudopregnant female foster animal. The offspring borne of this female foster animal will be a clone of the animal from which the cell (e.g., the somatic cell) is isolated.

Pharmaceutical Compositions

The POLYX nucleic acid molecules, POLYX proteins, and anti-POLYX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a POLYX protein or anti-POLYX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No.5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The nucleic acid molecules, proteins, protein homologues, and antibodies described herein can be used in one or more of the following methods: (i) screening assays; (ii) detection assays (e.g., chromosomal mapping, cell and tissue typing, forensic biology), (iii) predictive medicine (e.g., diagnostic assays, prognostic assays, monitoring clinical trials, and pharmacogenomics); and (iv) methods of treatment (e.g., therapeutic and prophylactic).

The isolated nucleic acid molecules of the present invention can be used to express POLYX protein (e.g., via a recombinant expression vector in a host cell in gene therapy applications), to detect POLYX mRNA (e.g., in a biological sample) or a genetic lesion in an POLYX gene, and to modulate POLYX activity, as described further, infra. In addition, the POLYX proteins can be used to screen drugs or compounds that modulate the POLYX protein activity or expression as well as to treat disorders characterized by insufficient or excessive production of POLYX protein or production of POLYX protein forms that have decreased or aberrant activity compared to POLYX wild-type protein. In addition, the anti-POLYX antibodies of the present invention can be used to detect and isolate POLYX proteins and modulate POLYX activity.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to POLYX proteins or have a stimulatory or inhibitory effect on, e.g., POLYX protein expression or POLYX protein activity. The invention also includes compounds identified in the screening assays described herein.

In one embodiment, the invention provides assays for screening candidate or test compounds which bind to or modulate the activity of the membrane-bound form of a POLYX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of POLYX protein, or a biologically-active portion thereof, on the cell surface is contacted with a test compound and the ability of the test compound to bind to a POLYX protein determined. The cell, for example, can of mammalian origin or a yeast cell. Determining the ability of the test compound to bind to the POLYX protein can be accomplished, for example, by coupling the test compound with a radioisotope or enzymatic label such that binding of the test compound to the POLYX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of POLYX protein, or a biologically-active portion thereof, on the cell surface with a known compound which binds POLYX to form an assay mixture, contacting the assay mixture with a test compound, and determining the ability of the test compound to interact with a POLYX protein, wherein determining the ability of the test compound to interact with a POLYX protein comprises determining the ability of the test compound to preferentially bind to POLYX protein or a biologically-active portion thereof as compared to the known compound.

In another embodiment, an assay is a cell-based assay comprising contacting a cell expressing a membrane-bound form of POLYX protein, or a biologically-active portion thereof, on the cell surface with a test compound and determining the ability of the test compound to modulate (e.g., stimulate or inhibit) the activity of the POLYX protein or biologically-active portion thereof. Determining the ability of the test compound to modulate the activity of POLYX or a biologically-active portion thereof can be accomplished, for example, by determining the ability of the POLYX protein to bind to or interact with a POLYX target molecule. As used herein, a “target molecule” is a molecule with which a POLYX protein binds or interacts in nature, for example, a molecule on the surface of a cell which expresses a POLYX interacting protein, a molecule on the surface of a second cell, a molecule in the extracellular milieu, a molecule associated with the internal surface of a cell membrane or a cytoplasmic molecule. An POLYX target molecule can be a non-POLYX molecule or a POLYX protein or polypeptide of the invention. In one embodiment, a POLYX target molecule is a component of a signal transduction pathway that facilitates transduction of an extracellular signal (e.g. a signal generated by binding of a compound to a membrane-bound POLYX molecule) through the cell membrane and into the cell. The target, for example, can be a second intercellular protein that has catalytic activity or a protein that facilitates the association of downstream signaling molecules with POLY.

Determining the ability of the POLYX protein to bind to or interact with a POLYX target molecule can be accomplished by one of the methods described above for determining direct binding. In one embodiment, determining the ability of the POLYX protein to bind to or interact with a POLYX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a POLYX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a POLYX protein or biologically-active portion thereof with a test compound and determining the ability of the test compound to bind to the POLYX protein or biologically-active portion thereof. Binding of the test compound to the POLYX protein can be determined either directly or indirectly as described above. In one such embodiment, the assay comprises contacting the POLYX protein or biologically-active portion thereof with a known compound which binds POLYX to form an assay mixture, contacting the assay mixture with a test compound, and determining the ability of the test compound to interact with a POLYX protein, wherein determining the ability of the test compound to interact with a POLYX protein comprises determining the ability of the test compound to preferentially bind to POLYX or biologically-active portion thereof as compared to the known compound.

In still another embodiment, an assay is a cell-free assay comprising contacting POLYX protein or biologically-active portion thereof with a test compound and determining the ability of the test compound to modulate (e.g. stimulate or inhibit) the activity of the POLYX protein or biologically-active portion thereof. Determining the ability of the test compound to modulate the activity of POLYX can be accomplished, for example, by determining the ability of the POLYX protein to bind to a POLYX target molecule by one of the methods described above for determining direct binding. In an alternative embodiment, determining the ability of the test compound to modulate the activity of POLYX protein can be accomplished by determining the ability of the POLYX protein further modulate a POLYX target molecule. For example, the catalytic/enzymatic activity of the target molecule on an appropriate substrate can be determined as described, supra.

In yet another embodiment, the cell-free assay comprises contacting the POLYX protein or biologically-active portion thereof with a known compound which binds POLYX protein to form an assay mixture, contacting the assay mixture with a test compound, and determining the ability of the test compound to interact with a POLYX protein, wherein determining the ability of the test compound to interact with a POLYX protein comprises determining the ability of the POLYX protein to preferentially bind to or modulate the activity of a POLYX target molecule.

The cell-free assays of the invention are amenable to use of both the soluble form or the membrane-bound form of POLYX protein. In the case of cell-free assays comprising the membrane-bound form of POLYX protein, it may be desirable to utilize a solubilizing agent such that the membrane-bound form of POLYX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either POLYX protein or its target molecule to facilitate separation of complexed from uncomplexed forms of one or both of the proteins, as well as to accommodate automation of the assay. Binding of a test compound to POLYX protein, or interaction of POLYX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-POLYX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or POLYX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of POLYX protein binding or activity determined using standard techniques.

Other techniques for immobilizing proteins on matrices can also be used in the screening assays of the invention. For example, either the POLYX protein or its target molecule can be immobilized utilizing conjugation of biotin and streptavidin. Biotinylated POLYX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with POLYX protein or target molecules, but which do not interfere with binding of the POLYX protein to its target molecule, can be derivatized to the wells of the plate, and unbound target or POLYX protein trapped in the wells by antibody conjugation. Methods for detecting such complexes, in addition to those described above for the GST-immobilized complexes, include immunodetection of complexes using antibodies reactive with the POLYX protein or target molecule, as well as enzyme-linked assays that rely on detecting an enzymatic activity associated with the POLYX protein or target molecule.

In another embodiment, modulators of POLYX protein expression are identified in a method wherein a cell is contacted with a candidate compound and the expression of POLYX mRNA or protein in the cell is determined. The level of expression of POLYX mRNA or protein in the presence of the candidate compound is compared to the level of expression of POLYX mRNA or protein in the absence of the candidate compound. The candidate compound can then be identified as a modulator of POLYX mRNA or protein expression based upon this comparison. For example, when expression of POLYX mRNA or protein is greater (i.e., statistically significantly greater) in the presence of the candidate compound than in its absence, the candidate compound is identified as a stimulator of POLYX mRNA or protein expression. Alternatively, when expression of POLYX mRNA or protein is less (statistically significantly less) in the presence of the candidate compound than in its absence, the candidate compound is identified as an inhibitor of POLYX mRNA or protein expression. The level of POLYX mRNA or protein expression in the cells can be determined by methods described herein for detecting POLYX mRNA or protein.

In yet another aspect of the invention, the POLYX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with POLYX (“POLYX-binding proteins” or “POLYX-bp”) and modulate POLYX activity. Such POLYX-binding proteins are also likely to be involved in the propagation of signals by the POLYX proteins as, for example, upstream or downstream elements of the POLYX pathway.

The two-hybrid system is based on the modular nature of most transcription factors, which consist of separable DNA-binding and activation domains. Briefly, the assay utilizes two different DNA constructs. In one construct, the gene that codes for POLYX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a POLYX-dependent complex, the DNA-binding and activation domains of the transcription factor are brought into close POLYX imity. This POLYX imity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with POLY.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, infra.

Chromosome Mapping

Once the sequence (or a portion of the sequence) of a gene has been isolated, this sequence can be used to map the location of the gene on a chromosome. This process is called chromosome mapping. Accordingly, portions or fragments of the POLYX sequences shown in SEQ ID NO:2n-1 (wherein n=1 to 14), or fragments or derivatives thereof, can be used to map the location of the POLYX genes, respectively, on a chromosome. The mapping of the POLYX sequences to chromosomes is an important first step in correlating these sequences with genes associated with disease.

Briefly, POLYX genes can be mapped to chromosomes by preparing PCR primers (preferably 15-25 bp in length) from the POLYX sequences. Computer analysis of the POLY, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the POLYX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the POLYX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, NY 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the POLYX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The POLYX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the POLYX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The POLYX sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted coding sequences, such as those in SEQ ID NO:2n-1 (wherein n=1 to 14) are used, a more appropriate number of primers for positive individual identification would be 500-2,000.

Use of Partial POLYX Sequences in Forensic Biology

DNA-based identification techniques can also be used in forensic biology. Forensic biology is a scientific field employing genetic typing of biological evidence found at a crime scene as a means for positively identifying, e.g., a perpetrator of a crime. To make such an identification, PCR technology can be used to amplify DNA sequences taken from very small biological samples such as tissues (e.g., hair or skin, or body fluids, e.g., blood, saliva, or semen found at a crime scene). The amplified sequence can then be compared to a standard, thereby allowing identification of the origin of the biological sample.

The sequences of the invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions of SEQ ID NO:2n-1 (where n=1 to 14) are particularly appropriate for this use as greater numbers of polymorphisms occur in the non-coding regions, making it easier to differentiate individuals using this technique. Examples of polynucleotide reagents include the POLYX sequences or portions thereof, e.g., fragments derived from the non-coding regions of one or more of SEQ ID NO:2n-1 (where n=1 to 14), having a length of at least 20 bases, preferably at least 30 bases.

The POLYX sequences described herein can further be used to provide polynucleotide reagents, e.g., labeled or label-able probes that can be used, for example, in an in situ hybridization technique, to identify a specific tissue (e.g., brain tissue, etc). This can be very useful in cases where a forensic pathologist is presented with a tissue of unknown origin. Panels of such POLYX probes can be used to identify tissue by species and/or by organ type.

In a similar fashion, these reagents, e.g., POLYX primers or probes can be used to screen tissue culture for contamination (i.e., screen for the presence of a mixture of different types of cells in a culture).

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining POLYX protein and/or nucleic acid expression as well as POLYX activity, in the context of a biological sample (e.g., blood, serum, cells, tissue) to thereby determine whether an individual is afflicted with a disease or disorder, or is at risk of developing a disorder, associated with aberrant POLYX expression or activity. The invention also provides for prognostic (or predictive) assays for determining whether an individual is at risk of developing a disorder associated with POLYX protein, nucleic acid expression or activity. For example, mutations in a POLYX gene can be assayed in a biological sample. Such assays can be used for prognostic or predictive purpose to thereby prophylactically treat an individual prior to the onset of a disorder characterized by or associated with POLYX protein, nucleic acid expression, or biological activity.

Another aspect of the invention provides methods for determining POLYX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.).

Yet another aspect of the invention pertains to monitoring the influence of agents (e.g., drugs, compounds) on the expression or activity of POLYX in clinical trials. These and other agents are described in further detail in the following sections.

Diagnostic Assays

An exemplary method for detecting the presence or absence of POLYX in a biological sample involves obtaining a biological sample from a test subject and contacting the biological sample with a compound or an agent capable of detecting POLYX protein or nucleic acid (e.g., mRNA, genomic DNA) that encodes POLYX protein such that the presence of POLYX is detected in the biological sample. An agent for detecting POLYX mRNA or genomic DNA is a labeled nucleic acid probe capable of hybridizing to POLYX mRNA or genomic DNA. The nucleic acid probe can be, for example, a full-length POLYX nucleic acid, such as the nucleic acid of SEQ ID NO:2n-1 (wherein n=1 to 14), or a portion thereof, such as an oligonucleotide of at least 15, 30, 50, 100, 250 or 500 nucleotides in length and sufficient to specifically hybridize under stringent conditions to POLYX mRNA or genomic DNA. Other suitable probes for use in the diagnostic assays of the invention are described herein.

An agent for detecting POLYX protein is an antibody capable of binding to POLYX protein, preferably an antibody with a detectable label. Antibodies can be polyclonal, or more preferably, monoclonal. An intact antibody, or a fragment thereof (e.g., F_{ab} or F_{(ab)2}) can be used. As utilized herein, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. As utilized herein, the term “biological sample” is intended to include tissues, cells and biological fluids isolated from a subject, as well as tissues, cells and fluids present within a subject. That is, the detection method of the invention can be used to detect POLYX mRNA, protein, or genomic DNA in a biological sample in vitro as well as in vivo. For example, in vitro techniques for detection of POLYX mRNA include Northern hybridizations and in situ hybridizations. In vitro techniques for detection of POLYX protein include enzyme linked immunosorbent assays (ELISAs), Western blots, immunoprecipitations, and immunofluorescence. In vitro techniques for detection of POLYX genomic DNA include Southern hybridizations. Furthermore, in vivo techniques for detection of POLYX protein include introducing into a subject a labeled anti-POLYX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting POLYX protein, mRNA, or genomic DNA, such that the presence of POLYX protein, mRNA or genomic DNA is detected in the biological sample, and comparing the presence of POLYX protein, mRNA or genomic DNA in the control sample with the presence of POLYX protein, mRNA or genomic DNA in the test sample.

The invention also encompasses kits for detecting the presence of POLYX in a biological sample. For example, the kit can comprise: a labeled compound or agent capable of detecting POLYX protein or mRNA in a biological sample; means for determining the amount of POLYX in the sample; and means for comparing the amount of POLYX in the sample with a standard. The compound or agent can be packaged in a suitable container. The kit can further comprise instructions for using the kit to detect POLYX protein or nucleic acid.

Prognostic Assays

The diagnostic methods described herein can furthermore be utilized to identify subjects having or at risk of developing a disease or disorder associated with aberrant POLYX expression or activity. For example, the assays described herein, such as the preceding diagnostic assays or the following assays, can be utilized to identify a subject having or at risk of developing a disorder associated with POLYX protein, nucleic acid expression or activity. Alternatively, the prognostic assays can be utilized to identify a subject having or at risk for developing a disease or disorder. Thus, the invention provides a method for identifying a disease or disorder associated with aberrant POLYX expression or activity in which a test sample is obtained from a subject and POLYX protein or nucleic acid (e.g., mRNA, genomic DNA) is detected, wherein the presence of POLYX protein or nucleic acid is diagnostic for a subject having or at risk of developing a disease or disorder associated with aberrant POLYX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant POLYX expression or activity. For example, such methods can be used to determine whether a subject can be effectively treated with an agent for a disorder. Thus, the invention provides methods for determining whether a subject can be effectively treated with an agent for a disorder associated with aberrant POLYX expression or activity in which a test sample is obtained and POLYX protein or nucleic acid is detected (e.g., wherein the presence of POLYX protein or nucleic acid is diagnostic for a subject that can be administered the agent to treat a disorder associated with aberrant POLYX expression or activity).

The methods of the invention can also be used to detect genetic lesions in a POLYX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a POLYX-protein, or the mis-expression of the POLYX gene. For example, such genetic lesions can be detected by ascertaining the existence of at least one of: (i) a deletion of one or more nucleotides from a POLYX gene; (ii) an addition of one or more nucleotides to a POLYX gene; (iii) a substitution of one or more nucleotides of a POLYX gene, (iv) a chromosomal rearrangement of a POLYX gene; (v) an alteration in the level of a messenger RNA transcript of a POLYX gene; (vi) aberrant modification of a POLYX gene, such as of the methylation pattern of the genomic DNA; (vii) the presence of a non-wild-type splicing pattern of a messenger RNA transcript of a POLYX gene; (viii) a non-wild-type level of a POLYX protein, (ix) allelic loss of a POLYX gene; and (x) inappropriate post-translational modification of a POLYX protein. As described herein, there are a large number of assay techniques known in the art which can be used for detecting lesions in a POLYX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the POLYX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a POLYX gene under conditions such that hybridization and amplification of the POLYX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a POLYX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in POLYX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in POLYX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the POLYX gene and detect mutations by comparing the sequence of the sample POLYX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. BioTechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the POLYX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type POLYX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in POLYX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a POLYX sequence, e.g., a wild-type POLYX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in POLYX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control POLYX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of apPOLYXimately 40 bp of high-melting GC-rich DNA by PCR. In a further embodiment, a temperature gradient is used in place of a denaturing gradient to identify differences in the mobility of control and sample DNA. See, e.g. Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a POLYX gene.

Furthermore, any cell type or tissue, preferably peripheral blood leukocytes, in which POLYX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on POLYX activity (e.g., POLYX gene expression), as identified by a screening assay described herein can be administered to individuals to treat (prophylactically or therapeutically) disorders (e.g., cancer or immune disorders associated with aberrant POLYX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of POLYX protein, expression of POLYX nucleic acid, or mutation content of POLYX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol. 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of POLYX protein, expression of POLYX nucleic acid, or mutation content of POLYX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a POLYX modulator, such as a modulator identified by one of the exemplary screening assays described herein.

Monitoring of Effects During Clinical Trials

Monitoring the influence of agents (e.g., drugs, compounds) on the expression or activity of POLYX (e.g., the ability to modulate aberrant cell proliferation and/or differentiation) can be applied not only in basic drug screening, but also in clinical trials. For example, the effectiveness of an agent determined by a screening assay as described herein to increase POLYX gene expression, protein levels, or upregulate POLYX activity, can be monitored in clinical trails of subjects exhibiting decreased POLYX gene expression, protein levels, or downregulated POLYX activity. Alternatively, the effectiveness of an agent determined by a screening assay to decrease POLYX gene expression, protein levels, or downregulate POLYX activity, can be monitored in clinical trails of subjects exhibiting increased POLYX gene expression, protein levels, or upregulated POLYX activity. In such clinical trials, the expression or activity of POLYX and, preferably, other genes that have been implicated in, for example, a cellular proliferation or immune disorder can be used as a “read out” or markers of the immune responsiveness of a particular cell.

By way of example, and not of limitation, genes, including POLY, that are modulated in cells by treatment with an agent (e.g., compound, drug or small molecule) that modulates POLYX activity (e.g., identified in a screening assay as described herein) can be identified. Thus, to study the effect of agents on cellular proliferation disorders, for example, in a clinical trial, cells can be isolated and RNA prepared and analyzed for the levels of expression of POLYX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of POLYX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a POLYX protein, mRNA, or genomic DNA in the pre-administration sample; (iii) obtaining one or more post-administration samples from the subject; (iv) detecting the level of expression or activity of the POLYX protein, mRNA, or genomic DNA in the post-administration samples; (v) comparing the level of expression or activity of the POLYX protein, mRNA, or genomic DNA in the pre-administration sample with the POLYX protein, mRNA, or genomic DNA in the post administration sample or samples; and (vi) altering the administration of the agent to the subject accordingly. For example, increased administration of the agent may be desirable to increase the expression or activity of POLYX to higher levels than detected, i.e., to increase the effectiveness of the agent. Alternatively, decreased administration of the agent may be desirable to decrease expression or activity of POLYX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant POLYX expression or activity. These methods of treatment will be discussed more fully, infra.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant POLYX expression or activity, by administering to the subject an agent that modulates POLYX expression or at least one POLYX activity. Subjects at risk for a disease that is caused or contributed to by aberrant POLYX expression or activity can be identified by, for example, any or a combination of diagnostic or prognostic assays as described herein. Administration of a prophylactic agent can occur prior to the manifestation of symptoms characteristic of the POLYX aberrancy, such that a disease or disorder is prevented or, alternatively, delayed in its progression. Depending upon the type of POLYX aberrancy, for example, a POLYX agonist or POLYX antagonist agent can be used for treating the subject. The appropriate agent can be determined based on screening assays described herein.

Therapeutic Methods

Another aspect of the invention pertains to methods of modulating POLYX expression or activity for therapeutic purposes. The modulatory method of the invention involves contacting a cell with an agent that modulates one or more of the activities of POLYX protein activity associated with the cell. An agent that modulates POLYX protein activity can be an agent as described herein, such as a nucleic acid or a protein, a naturally-occurring cognate ligand of a POLYX protein, a peptide, a POLYX peptidomimetic, or other small molecule. In one embodiment, the agent stimulates one or more POLYX protein activity. Examples of such stimulatory agents include active POLYX protein and a nucleic acid molecule encoding POLYX that has been introduced into the cell. In another embodiment, the agent inhibits one or more POLYX protein activity. Examples of such inhibitory agents include antisense POLYX nucleic acid molecules and anti-POLYX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a POLYX protein or nucleic acid molecule. In one embodiment, the method involves administering an agent (e.g., an agent identified by a screening assay described herein), or combination of agents that modulates (e.g., up-regulates or down-regulates) POLYX expression or activity. In another embodiment, the method involves administering a POLYX protein or nucleic acid molecule as therapy to compensate for reduced or aberrant POLYX expression or activity.

Stimulation of POLYX activity is desirable in situations in which POLYX is abnormally downregulated and/or in which increased POLYX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., pre-clampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The POLYX nucleic acids and proteins of the invention may be useful in a variety of potential prophylactic and therapeutic applications. By way of a non-limiting example, a cDNA encoding the POLYX protein of the invention may be useful in gene therapy, and the protein may be useful when administered to a subject in need thereof.

Both the novel nucleic acids encoding the POLYX proteins, and the POLYX proteins of the invention, or fragments thereof, may also be useful in diagnostic applications, wherein the presence or amount of the nucleic acid or the protein are to be assessed. These materials are further useful in the generation of antibodies which immunospecifically-bind to the novel substances of the invention for use in therapeutic or diagnostic methods.

The invention will be further illustrated in the following non-limiting examples.

### EXAMPLE 1

### Identification of Polyx Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### EXAMPLE 2

### Identification of Single Nucleotide Polymorphisms in Polyx Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### EXAMPLE 3

### Isolation of a POLY3 (CG51448-04) Nucleic Acid

The sequence of Acc. No. CG51448-04 was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

The laboratory cloning was performed using one or more of the methods summarized below:

A POLY3 nucleic acid was obtained by exon linking and extended by RACE as described below.

RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. The following human samples from different donors were used adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus for the RACE reaction. The sequences derived from these procedures were included in the SeqCalling Assembly process described in the preceding paragraph.

Exon Linking: The cDNA coding for the CG51448-04 sequence was cloned by the polymerase chain reaction (PCR) using the primers: 5′-GCCTCCCTACCTCATGGCGAC-3′(SEQ ID NO. 45) and 5′-CACATCGGGGAAGCGGTCAC-3′ (SEQ ID NO. 46). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

### EXAMPLE 4

### Quantitative Expression Analysis Polyx Nucleic Acids in Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (˜50 ng total or ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. CDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for 3-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan® PCR Master Mix for the PE Biosystems 7700, with 5 mM MgCl2, dNTPs (dA, G, C, U at 1:1:1:2 ratios), 0.25 U/ml AmpliTaq GoldTM (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca.=carcinoma,

*=established from metastasis,

met=metastasis,

s cell var=small cell variant,

non-s=non-sm=non-small,

squam=squamous,

pl. eff pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissue were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:128s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), I mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Thl, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD 110 cells were activated for 6 and 14 hours with approximately 5 ng/ml TNF alpha and 1 ng/ml IL-I beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

A. POLY1

Quantitative expression of POLY1 was assessed as described in Example 4 was assessed using the primer-probe set Ag764, described in Table 16A. Results of the RTQ-PCR runs are shown in Table 16B.

TABLE 16A

Probe Name: Ag764

| | | | | |
|---|---|---|---|---|
| | | | | Start |
| Primers | Sequences | TM | Length | Position |

| | | | | |
|---|---|---|---|---|
| Forward | 5′-AACGAGAAGCTGAAGGTGAACT-3′ (SEQ ID NO 48) | 59.6 | 22 | 1306 |
| Probe | FAM-5′-ACCCCAGAGTTCCTGTCACCTGAGGT-3′-TAMRA | 70.2 | 26 | 1333 |
| | (SEQ ID NO.:49) | | | |
| Reverse | 5′-TCGGAGATTTGGTCATAATTCA-3′ | 59.4 | 22 | 1361 |
| | (SEQ ID NO.:50) | | | |

TABLE 16B
Panel 1.3 and Panel 4D

| PANEL 1.3D | | PANEL 4D | |
|---|---|---|---|
| | Relative | | Relative |
| | Expression (%) | | Expression (%) |
| | 1.3dx4tm5495f_ | | 4Dtm2428f_ag |
| Tissue Name | ag764_b1 | Tissue Name | 764 |
| Liver adenocarcinoma | 100.0 | 93768_Secondary Th1_anti- | 6.6 |
| | | CD28/anti-CD3 | |
| Pancreas | 0.0 | 93769_Secondary Th2_anti- | 5.4 |
| | | CD28/anti-CD3 | |
| Pancreatic ca. CAPAN 2 | 0.0 | 93770_Secondary Tr1_anti- | 4.2 |
| | | CD28/anti-CD3 | |
| Adrenal gland | 0.0 | 93573_Secondary Th1_resting day | 0.0 |
| | | 4-6 in IL-2 | |
| Thyroid | 0.2 | 93572_Secondary Th2_resting day | 1.1 |
| | | 4-6 in IL-2 | |
| Salivary gland | 0.0 | 93571_Secondary TR1_resting day 4- | 0.0 |
| | | 6 in IL-2 | |
| Pituitary gland | 0.0 | 93568_primary Th1_anti-CD28/anti- | 5.0 |
| | | CD3 | |
| Brain (fetal) | 0.0 | 93569_primary Th2_anti-CD28/anti- | 2.6 |
| | | CD3 | |
| Brain (whole) | 0.0 | 93570_primary Tr1_anti-CD28/anti- | 4.4 |
| | | CD3 | |
| Brain (amygdala) | 0.0 | 93565_primary Th1_resting dy 4-6 | 7.4 |
| | | in IL-2 | |
| Brain (cerebellum) | 0.0 | 93566_primary Th2_resting dy 4-6 | 3.5 |
| | | in IL-2 | |
| Brain (hippocampus) | 0.0 | 93567_primary Tr1_resting dy 4-6 in | 3.0 |
| | | IL-2 | |
| Brain (substantia nigra) | 0.0 | 93351_CD45RA CD4 | 3.9 |
| | | lymphocyte_anti-CD28/anti-CD3 | |
| Brain (thalamus) | 0.0 | 93352_CD45RO CD4 | 2.6 |
| | | lymphocyte_anti-CD28/anti-CD3 | |
| Cerebral Cortex | 0.0 | 93251_CD8 Lymphocytes_anti- | 2.5 |
| | | CD28/anti-CD3 | |
| Spinal cord | 0.0 | 93353_chronic CD8 Lymphocytes | 4.7 |
| | | 2ry_resting dy 4-6 in IL-2 | |
| CNS ca. (glio/astro) U87- | 0.0 | 93574_chronic CD8 Lymphocytes | 2.3 |
| MG | | 2ry_activated CD3/CD28 | |
| CNS ca. (glio/astro) U- | 0.0 | 93354_CD4_none | 0.0 |
| 118-MG | | | |
| CNS ca. (astro) SW1783 | 0.0 | 93252_Secondary | 2.6 |
| | | Th1/Th2/Tr1_anti-CD95 CH11 | |
| CNS ca.* (neuro; met) | 0.0 | 93103_LAK cells_resting | 1.8 |
| SK-N-AS | | | |
| CNS ca. (astro) SF-539 | 0.0 | 93788_LAK cells_IL-2 | 3.3 |
| CNS ca. (astro) SNB-75 | 0.0 | 93787_LAK cells_IL-2+IL-12 | 3.4 |
| CNS ca. (gijo) SNB-19 | 0.0 | 93789_LAK cells_IL-2+IFN gamma | 2.6 |
| CNS ca. (glio) U251 | 0.0 | 93790_LAK cells_IL-2+IL-18 | 2.0 |
| CNS ca. (glio) SF-295 | 0.0 | 93104_LAK cells_PMA/ionomycin | 0.0 |
| | | and IL-18 | |
| Heart (fetal) | 0.0 | 93578_NK Cells IL-2_resting | 3.1 |
| Heart | 0.0 | 93109_Mixed Lymphocyte | 0.0 |
| | | Reaction_Two Way MLR | |
| Fetal Skeletal | 3.4 | 93110_Mixed Lymphocyte | 3.3 |
| | | Reaction_Two Way MLR | |
| Skeletal muscle | 32.2 | 93111_Mixed Lymphocyte | 1.2 |
| | | Reaction_Two Way MLR | |
| Bone marrow | 0.1 | 93112_Mononuclear Cells | 0.0 |
| | | (PBMCs)_resting | |
| Thymus | 0.0 | 93113_Mononuclear Cells | 4.5 |
| | | (PBMCs)_PWM | |
| Spleen | 0.0 | 93114_Mononuclear Cells | 7.0 |
| | | (PBMCs)_PHA-L | |
| Lymph node | 0.0 | 93249_Ramos (B cell)_none | 7.5 |
| Colorectal | 0.0 | 93250_Ramos (B cell)_ionomycin | 8.6 |
| Stomach | 0.0 | 93349_B lymphocytes_PWM | 2.1 |
| Small intestine | 0.0 | 93350_B lymphocytes_CD40L and | 4.6 |
| | | IL-4 | |
| Colon ca. SW480 | 0.0 | 92665_EOL-1 | 0.8 |
| | | (Eosinophil)_dbcAMP differentiated | |
| Colon ca.* (SW480 | 0.0 | 93248_EOL-1 | 0.0 |
| met)5W620 | | (Eosinophil)_dbcAMP/PMAionomy | |
| | | cin | |
| Colon ca. HT29 | 0.0 | 93356_Dendritic Cells_none | 0.0 |
| Colon ca. HCT-116 | 0.0 | 93355_Dendritic Cells_LPS 100 | 0.0 |
| | | ng/ml | |
| Colon ca. CaCo-2 | 0.0 | 93775_Dendritic Cells_anti-CD4O | 0.0 |
| 83219 CC Well to Mod | 0.0 | 93774_Monocytes_resting | 0.0 |
| Diff (OD03866) | | | |
| Colon ca. HCC-2998 | 0.0 | 93776_Monocytes LPS 50 ng/ml | 1.0 |
| Gastric ca.* (liver met) | 0.0 | 93581_Macrophages_resting | 0.5 |
| NCI-N87 | | | |
| Bladder | 0.0 | 93582_Macrophages_LPS 100 ng/ml | 0.0 |
| Trachea | 0.0 | 93098_HUVEC (Endothelial) none | 15.9 |
| Kidney | 0.0 | 93099_HUVEC | 47.6 |
| | | (Endothelial)_starved | |
| Kidney (fetal) | 0.0 | 93100_HUVEC (Endothelial)_IL-1b | 6.8 |
| Renal ca. 786-0 | 0.0 | 93779_HUVEC (Endothelial)_IFN | 4.3 |
| | | gamma | |
| Renal ca. A498 | 0.0 | 93102_HUVEC (Endothelial)_TNF | 1.3 |
| | | alpha +IFN gamma | |
| Renal ca. RXF 393 | 0.0 | 93101_HUVEC (Endothelial)_TNF | 4.4 |
| | | alpha +IL4 | |
| Renal ca. ACHN | 0.0 | 93781_HUVEC (Endothelial)_IL-11 | 8.3 |
| Renal ca. UO-31 | 0.0 | 93583_Lung Microvascular | 43.8 |
| | | Endothelial Cells_none | |
| Renal ca. TK-10 | 0.0 | 93584_Lung Microvascular | 14.0 |
| | | Endothelial Cells_TNFa (4 ng/ml) | |
| | | and IL1b (1 ng/ml) | |
| Liver | 0.0 | 92662_Microvascular Dermal | 100.0 |
| | | endothelium_none | |
| Liver (fetal) | 0.0 | 92663_Microsvasular Dermal | 10.4 |
| | | endothelium_TNFa (4 ng/ml) and | |
| | | IL1b(1 ng/ml) | |
| Liver ca. (hepatoblast) | 0.0 | 93773_Bronchial epithelium_TNFa | 0.0 |
| HepG2 | | (4 ng/ml) and IL1b (1 ng/ml) ** | |
| Lung | 0.0 | 93347_Small Airway | 0.0 |
| | | Epithelium_none | |
| Lung (fetal) | 0.0 | 93348_Small Airway | 0.0 |
| | | Epithelium_TNFa (4 ng/ml) and | |
| | | IL1b (1 ng/ml) | |
| Lung ca. (small cell) LX- | 0.0 | 92668_Coronery Artery | 17.0 |
| 1 | | SMC_resting | |
| Lung ca. (small cell) | 0.0 | 92669_Coronery Artery SMC_TNFa | 5.6 |
| NCI-H69 | | (4 ng/ml) and IL1b (1 ng/ml) | |
| Lung ca. (s.cell var.) | 0.0 | 93107_astrocytes_resting | 0.7 |
| SHP-77 | | | |
| Lung ca. (large cell)NCI- | 0.0 | 93108_astrocytes_TNFa (4 ng/ml) | 0.0 |
| H460 | | and IL1b (1 ng/ml) | |
| Lung ca. (non-sm. cell) | 0.0 | 92666_KU-812 (Basophil)_resting | 3.0 |
| A549 | | | |
| Lung ca. (non-s.cell) | 0.0 | 92667_KU-812 | 0.0 |
| NCI-H23 | | (Basophil)_PMA/ionoycin | |
| Lung ca. (non-s.cell) | 0.0 | 93579_CCD1106 | 2.5 |
| HOP-62 | | (Keratinocytes)_none | |
| Lung ca. (non-s.cl) NCI- | 0.0 | 93580_CCD1106 | 0.9 |
| H522 | | (Keratinocytes)_TNFa and IFNg** | |
| Lung ca. (squam.) SW | 0.0 | 93791_Liver Cirrhosis | 2.3 |
| 900 | | | |
| Lung ca. (squam.) NCl- | 0.0 | 93792_Lupus Kidney | 1.9 |
| H596 | | | |
| Mammary gland | 0.0 | 93577_NCI-H292 | 2.1 |
| Breast ca.* (p1. effusion) | 0.0 | 93358_NCI-H292_IL-4 | 3.1 |
| MCF-7 | | | |
| Breast ca.* (p1.ef) MDA- | 0.0 | 93360_NCI-H292_IL-9 | 3.6 |
| MB-231 | | | |
| Breast ca.* (p1. effusion) | 0.0 | 93359_NCI-H292_IL-13 | 5.3 |
| T47D | | | |
| Breast ca. BT-549 | 0.0 | 93357_NCI-H292_IFN gamma | 0.6 |
| Breast ca. MDA-N | 0.0 | 93777_HPAEC_- | 3.4 |
| Ovary | 0.0 | 93778_HPAEC_IL-1 beta/TNA | 1.6 |
| | | alpha | |
| Ovarian ca OVCAR-3 | 0.0 | 93254_Normal Human Lung | 0.0 |
| | | Fibroblast_none | |
| Ovarian ca. OVCAR-4 | 0.0 | 93253_Normal Human Lung | 0.0 |
| | | Fibroblast_TNFa (4 ng/ml) and IL- | |
| | | 1b (1 ng/ml) | |
| Ovarian ca. OVCAR-5 | 0.0 | 93257_Normal Human Lung | 0.0 |
| | | Fibroblast_IL-4 | |
| Ovarian ca OVCAR-8 | 0.0 | 93256_Normal Human Lung | 0.0 |
| | | Fibroblast_IL-9 | |
| Ovarian ca. IGROV-1 | 0.0 | 93255_Normal Human Lung | 0.0 |
| | | Fibroblast_IL-13 | |
| Ovarian ca.* (ascites) SK- | 0.0 | 93258_Normal Human Lung | 0.0 |
| OV-3 | | Fibroblast_IFN gamma | |
| Uterus | 0.0 | 93106_Dermal Fibroblasts | 5.6 |
| | | CCD1070_resting | |
| Placenta | 0.0 | 93361_Dermal Fibroblasts | 19.8 |
| | | CCD1070_TNF alpha 4 ng/ml | |
| Prostate | 0.0 | 93105_Dermal Fibroblasts | 5.7 |
| | | CCD1070_IL-1 beta 1 ng/ml | |
| Prostate ca.* (bone | 0.0 | 93772_dermal fibroblast_IFN | 4.7 |
| met)PC-3 | | gamma | |
| Testis | 0.0 | 93771_dermal fibroblast_IL-4 | 2.4 |
| Melanoma Hs688(A).T | 0.0 | 93259_IBD Colitis 1** | 0.9 |
| Melanoma* (met) | 0.0 | 93260_IBD Colitis 2 | 0.0 |
| Hs688(B).T | | | |
| Melanoma UACC-62 | 0.0 | 93261_IBD Crohns | 0.0 |
| Melanoma M14 | 0.0 | 735010_Colon_normal | 3.6 |
| Melanoma LOX IMVI | 0.0 | 735019_Lung_none | 3.0 |
| Melanoma* (met) SK- | 0.0 | 64028-1_Thymus_none | 0.0 |
| MEL-5 | | | |
| Adipose | 0.0 | 64030-1_Kidney_none | 3.8 |

As is shown in Table 16B (Panel 1.3D), expression of POLY1 seems to be highest in liver adenocarcinoma. However, this is an experimental artifact (as seen by the abnormal amplification profile for this sample) skewing the relative expression in other tissues and must be ignored. Expression of this gene is highest among normal tissues in skeletal muscle, where it is expressed at roughly 10-fold higher levels than fetal skeletal muscle. Therefore this gene may be used as a marker to differentiate between adult and fetal skeletal muscle. Significantly lower levels are seen in thyroid, bone marrow, adipose, testis, thalamus and cerebral cortex. Expression in other tissues is low to undetectable.

As is shown in Table 16B (Panel 4D), there is high expression of POLY1 in untreated endothelial cells including the microvascular endothelium, human umbilical vein endothelial cells (HUVECS) and in lung endothelial cells. This transcript is highly expressed in normal tissue and down regulated in activated endothelium. It could encode a protein important for a pathway that is involved in maintaining cellular homeostasis within a tissue. A protein therapeutic designed with the protein encoded for by this transcript could reduce or eliminate inflammation in endothelium. This type of therapeutic could serve as a treatment for asthma, allergy, psoriasis, arthritis and other inflammatory and autoimmune diseases in which activated endothelium plays a role.

B. POLY7

Quantitative expression of POLY7 was assessed using the primer-probe set Ag1212, described in Table 16C. Results of the RTQ-PCR runs are shown in Tables 16D and 16E.

TABLE 16C
Probe Name: Ag1212

| | | | | | Start |
|---|---|---|---|---|---|
| Primers | Sequences | | TM | Length | Position |
| Forward | 5′-GACCATAACAGCTGCAAACTCT-3′ | (SEQ ID NO:51) | 58.5 | 22 | 107 |
| Probe | TET-5′-TTCATGAACACTGTACTGGTTG | (SEQ ID NO.:52) | 65.1 | 26 | 149 |
| | CCTT-3′-TAMRA | | | | |
| Reverse | 5′-AGCCCTTCTGGTTCTTTGTG-3′ | (SEQ ID NO.:53) | 59.3 | 20 | 175 |

TABLE 16D
Panels 1.3D and 4D

| PANEL 1.3D | | PANEL 4D | |
|---|---|---|---|
| | Relative | | Relative |
| | Expression (%) | | Expression (%) |
| | 1.3dx4tm5357t | | 4dtm2067t_ag |
| Tissue Name | _ag1212_a2 | Tissue Name | 1212 |
| Liver adenocarcinoma | 17.1 | 93768_Secondary Th1_anti- | 0.1 |
| | | CD28/anti-CD3 | |
| Pancreas | 0.0 | 93769_Secondary Th2_anti- | 0.0 |
| | | CD28/anti-CD3 | |
| Pancreatic ca. CAPAN 2 | 3.9 | 93770_Secondary Tr1_anti- | 0.0 |
| | | CD28/anti-CD3 | |
| Adrenal gland | 0.0 | 93573_Secondary Th1_resting day 4- | 0.0 |
| | | 6 in IL-2 | |
| Thyroid | 0.0 | 93572_Secondary Th2_resting day 4- | 0.0 |
| | | 6 in IL-2 | |
| Salivary gland | 0.0 | 93571_Secondary Tr1_resting day 4- | 0.0 |
| | | 6 in IL-2 | |
| Pituitary gland | 9.8 | 93568_primary Th1_anti-CD28/anti- | 0.0 |
| | | CD3 | |
| Brain (fetal) | 100.0 | 93569_primary Th2_anti-CD28/anti- | 0.0 |
| | | CD3 | |
| Brain (whole) | 80.4 | 93570_primary Tr1_anti-CD28/anti- | 0.0 |
| | | CD3 | |
| Brain (amygdala) | 29.6 | 93565_primary Th1_resting dy 4-6 in | 0.0 |
| | | IL-2 | |
| Brain (cerebellum) | 99.4 | 93566_primary Th2_resting dy 4-6 in | 0.0 |
| | | IL-2 | |
| Brain (hippocampus) | 64.8 | 93567_primary Tr1_resting dy 4-6 in | 0.0 |
| | | IL-2 | |
| Brain (substantia nigra) | 10.2 | 93351_CD45RA CD4 | 0.7 |
| | | lymphocyte_anti-CD28/anti-CD3 | |
| Brain (thalamus) | 27.3 | 93352_CD45RO CD4 | 0.0 |
| | | lymphocyte_anti-CD28/anti-CD3 | |
| Cerebral Cortex | 30.8 | 93251_CD8 Lymphocytes_anti- | 0.0 |
| | | CD28/anti-CD3 | |
| Spinal cord | 2.6 | 93353_chronic CD8 Lymphocytes | 0.0 |
| | | 2ry_resting dy 4-6 in IL-2 | |
| CNS ca. (glio/astro) U87- | 38.4 | 93574_chronic CD8 Lymphocytes | 0.0 |
| MG | | 2ry_activated CD3/CD28 | |
| CNS ca. (glio/astro) U- | 13.0 | 93354_CD4_none | 0.0 |
| 118-MG | | | |
| CNS ca. (astro) SW1783 | 8.4 | 93252_Secondary Th1/Th2/Tr1_anti- | 0.0 |
| | | CD95 CH11 | |
| CNS ca.* (neuro; met) | 1.5 | 93103_LAK cells_resting | 0.0 |
| SK-N-AS | | | |
| CNS ca. (astro) SF-539 | 10.2 | 93788_LAK cells_IL-2 | 0.0 |
| CNS ca. (astro) SNB-75 | 9.0 | 93787_LAK cells_IL-2+IL-12 | 0.2 |
| CNS ca. (glio) SNB-19 | 0.0 | 93789_LAK cells_IL-2+IFN gamma | 0.0 |
| CNS ca. (glio) U251 | 13.7 | 93790_LAK cells_IL-2+IL-18 | 0.0 |
| CNS ca. (glio) SF-295 | 2.2 | 93104_LAK cells_PMA/ionomycin | 0.0 |
| | | and IL-18 | |
| Heart (fetal) | 0.0 | 93578_NK Cells IL-2_resting | 0.0 |
| Heart | 1.6 | 93109_Mixed Lymphocyte | 0.0 |
| | | Reaction_Two Way MLR | |
| Fetal Skeletal | 3.1 | 93110_Mixed Lymphocyte | 0.0 |
| | | Reaction_Two Way MLR | |
| Skeletal muscle | 0.0 | 93111_Mixed Lymphocyte | 0.0 |
| | | Reaction_Two Way MLR | |
| Bone marrow | 0.0 | 93112_Mononuclear Cells | 0.0 |
| | | (PBMCs)_resting | |
| Thymus | 0.0 | 93113_Mononuclear Cells | 0.0 |
| | | (PBMCs)_PWM | |
| Spleen | 0.0 | 93114_Mononuclear Cells | 0.0 |
| | | (PBMCs)_PHA-L | |
| Lymph node | 0.0 | 93249_Ramos (B cell)_none | 42.6 |
| Colorectal | 0.0 | 93250_Ramos (B cell)_ionomycin | 100.0 |
| Stomach | 0.0 | 93349_B lymphocytes_PWM | 0.3 |
| Small intestine | 7.2 | 93350_B lymphocytes_CD40L and | 3.1 |
| | | IL-4 | |
| Colon ca. SW480 | 0.0 | 92665_EOL-1 (Eosinophil)_dbcAMP | 0.0 |
| | | differentiated | |
| Colon ca.* (SW480 | 0.0 | 93248_EOL-1 | 0.0 |
| met)SW620 | | (Eosinophil)_dbcAMP/PMAionomyci | |
| | | n | |
| Colon ca. HT29 | 0.0 | 93356_Dendritic Cells_none | 0.2 |
| Colon ca. HCT-116 | 0.0 | 93355_Dendritic Cells_LPS 100 | 0.0 |
| | | ng/ml | |
| Colon ca. CaCo-2 | 0.0 | 93775_Dendritic Cells_anti-CD40 | 0.0 |
| 83219 CC Well to Mod | 0.0 | 93774_Monocytes_resting | 0.1 |
| Diff (OD03866) | | | |
| Colon ca. HCC-2998 | 0.0 | 93776_Monocytes_LPS 50 ng/ml | 0.1 |
| Gastric ca.* (liver met) | 0.0 | 93581_Macrophages_resting | 0.9 |
| NCI-N87 | | | |
| Bladder | 4.6 | 93582_Macrophages_LPS 100 ng/ml | 0.0 |
| Trachea | 0.0 | 93098_HUVEC (Endothelial)_none | 3.8 |
| Kidney | 0.0 | 93099_HUVEC (Endothelial)_starved | 6.7 |
| Kidney (fetal) | 0.0 | 93100_HUVEC (Endothelial)_IL-1b | 2.3 |
| Renal ca. 786-0 | 0.0 | 93779_HUVEC (Endolhelial)_IFN | 2.2 |
| | | gamma | |
| Renal ca. A498 | 6.8 | 93102_HUVEC (Endothelial)_TNF | 0.3 |
| | | alpha +IFN gamma | |
| Renal ca. RXF 393 | 0.0 | 93101_HUVEC (Endothelial)_TNF | 1.0 |
| | | alpha +IL4 | |
| Renal ca. ACHN | 0.0 | 93781_HUVEC (Endothelial)_IL-11 | 2.1 |
| Renal ca. UO-31 | 7.4 | 93583_Lung Microvascular | 6.8 |
| | | Endothelial Cells_none | |
| Renal ca. TK-10 | 0.0 | 93584_Lung Microvascular | 1.9 |
| | | Endothelial Cells_TNFa (4 ng/ml) | |
| | | and IL1b (1 ng/ml) | |
| Liver | 3.1 | 92662_Microvascular Dermal | 8.4 |
| | | endothelium_none | |
| Liver (fetal) | 0.0 | 92663_Microsvasular Dermal | 1.6 |
| | | endothelium_TNFa (4 ng/ml) and | |
| | | IL1b (1 ng/ml) | |
| Liver ca. (hepatoblast) | 0.0 | 93773_Bronchial epithelium_TNFa (4 | 1.6 |
| HepG2 | | ng/ml) and IL1b (1 ng/ml) ** | |
| Lung | 0.0 | 93347_Small Airway | 0.2 |
| | | Epithelium_none | |
| Lung (fetal) | 0.0 | 93348_Small Airway | 2.8 |
| | | Epithelium_TNFa (4 ng/ml) and IL1b | |
| | | (1 ng/ml) | |
| Lung ca. (small cell) LX- | 0.0 | 92668_Coronery Artery SMC_resting | 1.7 |
| 1 | | | |
| Lung ca. (small cell) | 1.8 | 92669_Coronery Artery SMC_TNFa | 0.6 |
| NCI-H69 | | (4 ng/ml) and IL1b (1 ng/ml) | |
| Lung ca. (s.cell var.) | 2.1 | 93107_astrocytes_resting | 0.9 |
| SHP-77 | | | |
| Lung ca. (large cell)NCI- | 0.0 | 93108_astrocytes_TNFa (4 ng/ml) | 0.7 |
| H460 | | and IL1b (1 ng/ml) | |
| Lung ca. (non-sm. cell) | 0.0 | 92666_KU-812 (Basophil)_resting | 0.0 |
| A549 | | | |
| Lung ca. (non-s.cell) | 2.5 | 92667_KU-812 | 0.0 |
| NCI-H23 | | (Basophil)_PMA/ionoycin | |
| Lung ca(non-s.cell) | 15.4 | 93579_CCD1106 | 3.0 |
| HOP-62 | | (Keratinocytes)_none | |
| Lung ca. (non-s.cl) NCI- | 3.6 | 93580_CCD1106 | 11.7 |
| H522 | | (Keratinocytes)_TNFa and IFNg * * | |
| Lung ca. (squam.) SW | 0.0 | 93791_Liver Cirrhosis | 1.5 |
| 900 | | | |
| Lung ca. (squam.) NCI- | 21.4 | 93792_Lupus Kidney | 0.0 |
| H596 | | | |
| Mammary gland | 5.1 | 93577_NCI-H292 | 0.0 |
| Breast ca.* (pl. effusion) | 0.0 | 93358_NCI-H292_IL-4 | 0.5 |
| MCF-7 | | | |
| Breast ca.* (pl.ef) MDA- | 0.0 | 93360_NCI-H292_IL-9 | 0.1 |
| MB-231 | | | |
| Breast ca.* (pl. effusion) | 0.0 | 93359_NCI-H292_IL-13 | 0.2 |
| T47D | | | |
| Breast ca BT-549 | 0.0 | 93357_NCI-H292_IFN gamma | 0.0 |
| Breast ca. MDA-N | 0.0 | 93777_HPAEC_- | 7.6 |
| Ovary | 0.0 | 93778_HPAEC_IL-1 beta/TNA alpha | 2.0 |
| Ovarian ca. OVCAR-3 | 40.1 | 93254_Normal Human Lung | 0.3 |
| | | Fibroblast_none | |
| Ovarian ca. OVCAR-4 | 0.0 | 93253_Normal Human Lung | 0.0 |
| | | Fibroblast_TNFa (4 ng/ml) and IL-1b | |
| | | (1 ng/ml) | |
| Ovarian ca. OVCAR-5 | 5.5 | 93257_Normal Human Lung | 0.1 |
| | | Fibroblast_IL-4 | |
| Ovarian ca. OVCAR-8 | 4.4 | 93256_Normal Human Lung | 0.3 |
| | | Fibroblast_IL-9 | |
| Ovarian ca. IGROV-1 | 0.0 | 93255_Normal Human Lung | 1.4 |
| | | Fibroblast_IL-13 | |
| Ovarian ca.* (ascites) SK- | 0.0 | 93258_Normal Human Lung | 0.0 |
| OV-3 | | Fibroblast_IFN gamma | |
| Uterus | 0.0 | 93106_Dermal Fibroblasts | 11.4 |
| | | CCD1070_resting | |
| Placenta | 0.0 | 93361_Dermal Fibroblasts | 6.7 |
| | | CCD1070_TNF alpha 4 ng/ml | |
| Prostate | 2.6 | 93105_Dermal Fibroblasts | 4.5 |
| | | CCD1070_IL-1 beta 1 ng/ml | |
| Prostate ca.* (bone | 10.1 | 93772_dermal fibroblast_IFN gamma | 0.8 |
| met)PC-3 | | | |
| Testis | 3.0 | 93771_dermal fibroblast_IL-4 | 1.0 |
| Melanoma Hs688(A).T | 6.2 | 93259_IBD Colitis 1 ** | 2.1 |
| Melanoma* (met) | 18.9 | 93260_IBD Colitis 2 | 0.0 |
| Hs688(B).T | | | |
| Melanoma UACC-62 | 10.3 | 93261_IBD Crohns | 0.2 |
| Melanoma M14 | 85.4 | 735010_Colon_normal | 0.0 |
| Melanoma LOX IMVI | 23.3 | 735019_Lung_none | 0.0 |
| Melanoma* (met) SK- | 9.6 | 64028-1_Thymus_none | 0.5 |
| MEL-5 | | | |
| Adipose | 2.0 | 64030-1_Kidney_none | 0.0 |

TABLE 16E
Panels 2D and 3D

| PANEL 2D | | PANEL 3D | |
|---|---|---|---|
| | Relative | | Relative |
| | Expression (%) | | Expression (%) |
| | 2Dx4tm5023t_a | | 3dtm4929t_ag |
| Tissue Name | g1212_b1 | Tissue Name | 1212 |
| Normal Colon | 37.4 | 94905_Daoy_Medulloblastoma/Cereb | 2.6 |
| GENPAK 061003 | | ellum_sscDNA | |
| 83219 CC Well to Mod | 0.5 | 94906_TE671_Medulloblastom/Cere | 0.0 |
| Diff (OD03866) | | bellum_sscDNA | |
| 83220 CC NAT | 0.0 | 94907_D283 | 0.0 |
| (OD03866) | | Med_Medulloblastoma/Cerebellum_s | |
| | | scDNA | |
| 83221 CC Gr.2 | 0.0 | 94908_PFSK-1_Primitive | 10.6 |
| rectosigmoid | | Neuroectodermal/Cerebellum_sscDN | |
| (OD03868) | | A | |
| 83222 CC NAT | 0.0 | 94909_XF-498_CNS_sscDNA | 81.8 |
| (OD03868) | | | |
| 83235 CC Mod Diff | 0.0 | 94910_SNB- | 8.8 |
| (OD03920) | | 78_CNS/glioma_sscDNA | |
| 83236 CC NAT | 0.2 | 94911_SF- | 11.9 |
| (OD03920) | | 268_CNS/glioblastoma_sscDNA | |
| 83237 CC Gr.2 ascend | 0.0 | 94912_T98G_Glioblastoma_sscDNA | 1.8 |
| colon (OD03921) | | | |
| 83238 CC NAT | 0.0 | 96776_SK-N-SH_Neuroblastoma | 27.7 |
| (OD03921) | | (metastasis)_sscDNA | |
| 83241 CC from Partial | 1.4 | 94913_SF- | 0.0 |
| Hepatectomy | | 295_CNS/glioblastoma_sscDNA | |
| (OD04309) | | | |
| 83242 Liver NAT | 2.0 | 94914_Cerebellum_sscDNA | 84.7 |
| (OD04309) | | | |
| 87472 Colon mets to | 22.0 | 96777_Cerebellum_sscDNA | 7.1 |
| lung (OD04451-01) | | | |
| 87473 Lung NAT | 0.0 | 94916_NCI-H292_Mucoepidermoid | 1.0 |
| (OD04451-02) | | lung carcinoma_sscDNA | |
| Normal Prostate | 0.0 | 94917_DMS-114_Small cell lung | 0.0 |
| Clontech A+6546-1 | | cancer_sscDNA | |
| 84140 Prostate Cancer | 0.0 | 94918_DMS-79_Small cell lung | 38.4 |
| (OD04410) | | cancer/neuroendocrine_sscDNA | |
| 84141 Prostate NAT | 0.0 | 94919_NCI-H146_Small cell lung | 0.0 |
| (OD04410) | | cancer/neuroendocrine_sscDNA | |
| 87073 Prostate Cancer | 0.0 | 94920_NCI-H526_Small cell lung | 24.5 |
| (OD04720-01) | | cancer/neuroendocrine_sscDNA | |
| 87074 Prostate NAT | 0.0 | 94921_NCI-N417_Small cell lung | 9.4 |
| (OD04720-02) | | cancer/neuroendocrine_sscDNA | |
| Normal Lung GENPAK | 0.0 | 94923_NCI-H82_Small cell lung | 20.0 |
| 061010 | | cancer/neuroendocrine_sscDNA | |
| 83239 Lung Met to | 0.0 | 94924_NCI-H157_Squamous cell | 39.2 |
| Muscle (OD04286) | | lung cancer (metastasis)_sscDNA | |
| 83240 Muscle NAT | 0.0 | 94925_NCI-H1155_Large cell lung | 0.0 |
| (OD04286) | | cancer/neuroendocrine_sscDNA | |
| 84136 Lung Malignant | 0.2 | 94926_NCI-H1299_Large cell lung | 60.7 |
| Cancer (OD03126) | | cancer/neuroendocrine_sscDNA | |
| 84137 Lung NAT | 0.1 | 94927_NCI-H727_Lung | 0.0 |
| (OD03126) | | carcinoid_sscDNA | |
| 84871 Lung Cancer | 5.2 | 94928_NCI-UMC-11_Lung | 0.0 |
| (OD04404) | | carcinoid_sscDNA | |
| 84872 Lung NAT | 0.7 | 94929_LX-1_Small cell lung | 0.0 |
| (OD04404) | | cancer_sscDNA | |
| 84875 Lung Cancer | 0.0 | 94930_Colo-205_Colon | 0.0 |
| (OD04565) | | cancer_sscDNA | |
| 84876 Lung NAT | 0.0 | 94931_KM12_Colon cancer_sscDNA | 5.8 |
| (OD04565) | | | |
| 85950 Lung Cancer | 0.0 | 94932_KM20L2_Colon | 0.0 |
| (OD04237-01) | | cancer_sscDNA | |
| 85970 Lung NAT | 0.0 | 94933_NCI-H716_Colon | 0.0 |
| (OD04237-02) | | cancer_sscDNA | |
| 83255 Ocular Mel Met | 0.0 | 94935_SW-48_Colon | 0.0 |
| to Liver (OD04310) | | adenocarcinoma_sscDNA | |
| 83256 Liver NAT | 0.0 | 94936_SW1116_Colon | 0.0 |
| (OD04310) | | adenocarcinoma_sscDNA | |
| 84139 Melanoma Mets | 0.0 | 94937_LS 174T_Colon | 0.0 |
| to Lung (OD04321) | | adenocarcinoma_sscDNA | |
| 84138 Lung NAT | 0.0 | 94938_SW-948_Colon | 2.2 |
| (OD04321) | | adenocarcinoma_sscDNA | |
| Normal Kidney | 0.0 | 94939_SW-480_Colon | 0.0 |
| GENPAK 061008 | | adenocarcinoma_sscDNA | |
| 83786 Kidney Ca, | 0.2 | 94940_NCI-SNU-5_Gastric | 2.0 |
| Nuclear grade 2 | | carcinoma_sscDNA | |
| (OD04338) | | | |
| 83787 Kidney NAT | 5.5 | 94941_KATO III_Gastric | 68.8 |
| (OD04338) | | carcinoma_sscDNA | |
| 83788 Kidney Ca | 1.8 | 94943_NCI-SNU-16_Gastric | 0.0 |
| Nuclear grade ½ | | carcinoma_sscDNA | |
| (OD04339) | | | |
| 83789 Kidney NAT | 0.1 | 94944_NCI-SNU-1_Gastric | 13.8 |
| (OD04339) | | carcinoma_sscDNA | |
| 83790 Kidney Ca, Clear | 0.0 | 94946_RF-1_Gastric | 0.0 |
| cell type (OD04340) | | adenocarcinoma_sscDNA | |
| 83791 Kidney NAT | 0.0 | 94947_RF-48_Gastric | 0.0 |
| (OD04340) | | adenocarcinoma_sscDNA | |
| 83792 Kidney Ca, | 0.0 | 96778_MKN-45_Gastric | 0.0 |
| Nuclear grade 3 | | carcinoma_sscDNA | |
| (OD04348) | | | |
| 83793 Kidney NAT | 0.0 | 94949_NCI-N87_Gastric | 0.0 |
| (OD04348) | | carcinoma_sscDNA | |
| 87474 Kidney Cancer | 0.0 | 94951_OVCAR-5_Ovarian | 0.0 |
| (OD04622-01) | | carcinoma_sscDNA | |
| 87475 Kidney NAT | 0.0 | 94952_RL95-2_Uterine | 0.0 |
| (OD04622-03) | | carcinoma_sscDNA | |
| 85973 Kidney Cancer | 0.0 | 94953_HelaS3_Cervical | 0.0 |
| (OD04450-01) | | adenocarcinoma_sscDNA | |
| 85974 Kidney NAT | 0.0 | 94954_Ca Ski_Cervical epidermoid | 40.6 |
| (OD04450-03) | | carcinoma (metastasis)_sscDNA | |
| Kidney Cancer | 0.0 | 94955_ES-2_Ovarian clear cell | 29.7 |
| Clontech 8120607 | | carcinoma_sscDNA | |
| Kidney NAT Clontech | 0.0 | 94957_Ramos/6h stim_″; Stimulated | 4.5 |
| 8120608 | | with PMA/ionomycin 6h_sscDNA | |
| Kidney Cancer | 0.3 | 94958_Ramos/14h stim_″; Stimulated | 9.4 |
| Clontech 8120613 | | with PMA/ionomycin 14h_sscDNA | |
| Kidney NAT Clontech | 9.0 | 94962_MEG-01_Chronic | 1.9 |
| 8120614 | | myelogenous leukemia | |
| | | (megokaryoblast)_sscDNA | |
| Kidney Cancer | 1.2 | 94963_Raji_Burkitt's | 9.8 |
| Clontech 9010320 | | lymphoma_sscDNA | |
| Kidney NAT Clontech | 0.0 | 94964_Daudi_Burkitt's | 100.0 |
| 9010321 | | lymphoma_sscDNA | |
| Normal Uterus | 0.0 | 94965_U266_B-cell | 0.0 |
| GENPAK 061018 | | plasmacytoma/myeloma_sscDNA | |
| Uterus Cancer | 0.0 | 94968_CA46_Burkitt's | 2.0 |
| GENPAK 064011 | | lymphoma_sscDNA | |
| Normal Thyroid | 0.0 | 94970_RL_non-Hodgkin's B-cell | 0.0 |
| Clontech A+6570-1 | | lymphoma_sscDNA | |
| Thyroid Cancer | 0.0 | 94972 JM1_pre-B-cell | 0.0 |
| GENPAK 064010 | | lymphoma/leukemia_sscDNA | |
| Thyroid Cancer | 0.0 | 94973_Jurkat_T cell | 0.0 |
| INVITROGEN | | leukemia_sscDNA | |
| A302152 | | | |
| Thyroid NAT | 0.0 | 94974_TF- | 1.6 |
| INVITROGEN | | 1_Erythroleukemia_sscDNA | |
| A302153 | | | |
| Normal Breast | 0.5 | 94975_HUT 78_T-cell | 0.0 |
| GENPAK 061019 | | lymphoma_sscDNA | |
| 84877 Breast Cancer | 8.2 | 94977_U937_Histiocytic | 0.0 |
| (OD04566) | | lymphoma_sscDNA | |
| 85975 Breast Cancer | 0.1 | 94980_KU-812_Myelogenous | 0.0 |
| (OD04590-01) | | leukemia_sscDNA | |
| 85976 Breast Cancer | 0.0 | 94981_769-P_Clear cell renal | 0.0 |
| Mets (OD04590-03) | | carcinoma_sscDNA | |
| 87070 Breast Cancer | 0.0 | 94983_Caki-2_Clear cell renal | 0.0 |
| Metastasis (OD04655- | | carcinoma_sscDNA | |
| 05) | | | |
| GENPAK Breast | 1.3 | 94984_SW 839_Clear cell renal | 0.0 |
| Cancer 064006 | | carcinoma_sscDNA | |
| Breast Cancer Res. | 1.3 | 94986_G401_Wilms' tumor_sscDNA | 0.0 |
| Gen. 1024 | | | |
| Breast Cancer Clontech | 0.0 | 94987_Hs766T_Pancreatic carcinoma | 30.8 |
| 9100266 | | (LN metastasis)_sscDNA | |
| Breast NAT Clontech | 0.0 | 94988_CAPAN-1_Pancreatic | 6.8 |
| 9100265 | | adenocarcinoma (liver | |
| | | metastasis)_sscDNA | |
| Breast Cancer | 0.0 | 94989_SU86.86_Pancreatic | 50.7 |
| INVITROGEN | | carcinoma (liver metastasis)_sscDNA | |
| A209073 | | | |
| Breast NAT | 0.0 | 94990_BxPC-3_Pancreatic | 26.8 |
| INVITROGEN | | adenocarcinoma_sscDNA | |
| A2090734 | | | |
| Normal Liver | 0.4 | 94991_HPAC_Pancreatic | 4.4 |
| GENPAK 061009 | | adenocarcinoma_sscDNA | |
| Liver Cancer GENPAK | 0.1 | 94992_MIA PaCa-2_Pancreatic | 10.2 |
| 064003 | | carcinoma_sscDNA | |
| Liver Cancer Research | 5.0 | 94993_CFPAC-1_Pancreatic ductal | 3.8 |
| Genetics RNA 1025 | | adenocarcinoma_sscDNA | |
| Liver Cancer Research | 0.1 | 94994_PANC-1_Pancreatic | 12.7 |
| Genetics RNA 1026 | | epithelioid ductal carcinoma_sscDNA | |
| Paired Liver Cancer | 1.2 | 94996_T24_Bladder carcinma | 33.4 |
| Tissue Research | | (transitional cell)_sscDNA | |
| Genetics RNA 6004-T | | | |
| Paired Liver Tissue | 0.0 | 94997_5637_Bladder | 24.1 |
| Research Genetics | | carcinoma_sscDNA | |
| RNA 6004-N | | | |
| Paired Liver Cancer | 0.0 | 94998_HT-1197_Bladder | 19.9 |
| Tissue Research | | carcinoma_sscDNA | |
| Genetics RNA 6005-T | | | |
| Paired Liver Tissue | 0.0 | 94999_UM-UC-3_Bladder carcinoma | 5.1 |
| Research Genetics | | (transitional cell)_sscDNA | |
| RNA 6005-N | | | |
| Normal Bladder | 0.0 | 95000_A204_Rhabdomyosarcoma_ss | 0.0 |
| GENPAK 061001 | | CDNA | |
| Bladder Cancer | 0.2 | 95001_HT- | 95.3 |
| Research Genetics | | 1080_Fibrosarcoma_sscDNA | |
| RNA 1023 | | | |
| Bladder Cancer | 0.0 | 95002_MG-63_Osteosarcoma | 0.0 |
| INVITROGEN | | (bone)_sscDNA | |
| A302173 | | | |
| 87071 Bladder Cancer | 0.1 | 95003_SK-LMS-1_Leiomyosarcoma | 35.8 |
| (OD04718-01) | | (vulva)_sscDNA | |
| 87072 Bladder Normal | 0.1 | 95004_SJRH30_Rhabdomyosarcoma | 13.6 |
| Adjacent (OD04718- | | (met to bone marrow)_sscDNA | |
| 03) | | | |
| Normal Ovary Res. | 0.4 | 95005_A431_Epidermoid | 0.0 |
| Gen. | | carcinoma_sscDNA | |
| Ovarian Cancer | 100.0 | 95007_WM266- | 12.3 |
| GENPAK 064008 | | 4_Melanoma_sscDNA | |
| 87492 Ovary Cancer | 21.2 | 95010_DU 145_Prostate carcinoma | 0.0 |
| (OD04768-07) | | (brain metastasis)_sscDNA | |
| 87493 Ovary NAT | 20.5 | 95012_MDA-MB-468_Breast | 5.1 |
| (OD04768-08) | | adenocarcinoma_sscDNA | |
| Normal Stomach | 16.6 | 95013_SCC-4_Squamous cell | 0.0 |
| GENPAK 061017 | | carcinoma of tongue_sscDNA | |
| Gastric Cancer | 8.8 | 95014_SCC-9_Squamous cell | 0.0 |
| Clontech 9060358 | | carcinoma of tongue_sscDNA | |
| NAT Stomach Clontech | 7.1 | 95015_SCC-15_Squamous cell | 0.0 |
| 9060359 | | carcinoma of tongue_sscDNA | |
| Gastric Cancer | 6.4 | 95017_CAL 27_Squamous cell | 0.0 |
| Clontech 9060395 | | carcinoma of tongue_sscDNA | |
| NAT Stomach Clontech | 8.6 | | |
| 9060394 | | | |
| Gastric Cancer | 2.9 | | |
| Clontech 9060397 | | | |
| NAT Stomach Clontech | 1.7 | | |
| 9060396 | | | |
| Gastric Cancer | 5.8 | | |
| GENPAK 064005 | | | |

As is shown in Table 16D (Panel 1.3D), the expression of POLY7 is highest in brain, with low level expression in various other tissues. Importantly, it is present in almost all melanomas, but is somewhat reduced in brain cancers relative to normal brain tissue. Thus it may be of use in screening/diagnosing melanoma. Currently one member of the S100 family of proteins, S100B, of which calgizzarin is also a member, is used as a diagnostic marker for melanoma. Some calgizzarins are differentially expressed relative to the mitotic state of the cells; thus these may play a role in neuro-development in the brain.

As is shown in Table 16D (Panel 4D), the expression of POLY7 is detected only in the Ramos B cell line and in activated keratinocytes. Therapeutics to this molecule may reduce or inhibit inflammation in skin due to psoriasis, delayed type hypersensitivity, irritation or infection. Based on the expression of this molecule on the transformed B cell line (Ramos), it may also serve as a marker for B cell malignancies.

As is shown in Table 16E (Panel 2D), in contrast to panel 1.3D there appears to be no, or very low, expression in melanoma tissue samples in panel 2D. This likely reflects the fact that the melanoma specimens on panel 2D are derived from ocular melanomas and not cutaneous melanomas. Thus, the expression of POLY7 may be useful in the distinction between ocular and cutaneous melanoma.

As is shown in Table 16E (Panel 3D), the expression of POLY7 in brain concurs with expression pattern in panel 1.3D. This gene is expressed in a variety of cancers, being highly expressed in Burkitt's lymphoma, with lower levels in fibrosarcoma, gastric carcinoma, leiomyosarcoma, cervical and ovarian carcinomas, bladder carcinoma and pancreatic carcinomas.

C. POLY8

Quantitative expression of POLY8 was assessed using the primer-probe sets Ag1084/Ag1147 (identical sequences), described in Table 16F.

TABLE 16F
Probe Name: Ag1084/Ag1147

| | | | | | Start |
|---|---|---|---|---|---|
| Primers | Sequences | | TM | Length | Position |
| Forward | 5′-CTCAAGTGATCCACCTGACTTT-3′ | (SEQ ID NO.:54) | 58.3 | 22 | 23 |
| Probe | FAM-5′-CCTCCCTCCCGAAGAGAGATA | (SEQ ID NO.:55) | 69.1 | 26 | 46 |
| | AGTCG-3′TAMRA | | | | |
| Reverse | 5′-TTTGGAAGGCAGTGGATTTT-3′ | (SEQ ID NO.:56) | 59.5 | 20 | 100 |

Expression of POLY8 was low/undetectable in panels 1.2 and 4D (Ct>35).

D. POLY9

Quantitative expression of POLY9 was assessed using the primer-probe set Agl 158, described in Table 16G. Results of the RTQ-PCR runs are shown in Table 16H.

TABLE 16G
Probe Name: Ag1158

| | | | | | Start |
|---|---|---|---|---|---|
| Primers | Sequences | | TM | Length | Position |
| Forward | 5′-GGACAGGGTGACTAGGTCATCT-3′ | (SEQ ID NO.:57) | 59.5 | 22 | 50 |
| Probe | FAM-5′-CAAACATGCTGTATGTCAATGG | (SEQ ID NO.:58) | 67.9 | 26 | 73 |
| | CACA-3′-TAMRA | | | | |
| Reverse | 5′-GCTGACGACCAGTTGTATGG-3′ | (SEQ ID NO.:59) | 58.2 | 20 | 115 |

TABLE 16H
Panels 1.2 and 4D

| PANEL 1.2 | | PANEL 4D | |
|---|---|---|---|
| | Rel. Expr., | | Rel. Expr., |
| | % | | % |
| | 1.2tm1353f_{—} | | 4dtm2013f |
| Tissue Name | ag1158 | Tissue Name | _ag1158 |
| Endothelial cells | 2.7 | 93768_Secondary Th1_anti-CD28/anti-CD3 | 0.0 |
| Endothelial cells | 1.4 | 93769_Secondary Th2_anti-CD28/anti-CD3 | 0.0 |
| (treated) | | | |
| Pancreas | 0.2 | 93770_Secondary Tr1_anti-CD28/anti-CD3 | 0.0 |
| Pancreatic ca. | 0.0 | 93573_Secondary Th1_resting day 4-6 in IL-2 | 0.0 |
| CAPAN 2 | | | |
| Adrenal Gland (new | 14.5 | 93572_Secondary Th2_resting day 4-6 in LL-2 | 0.0 |
| lot*) | | | |
| Thyroid | 1.9 | 93571_Secondary Tr1_resting day 4-6 in IL-2 | 0.0 |
| Salivary gland | 11.0 | 93568_primary Th1_anti-CD28/anti-CD3 | 0.0 |
| Pituitary gland | 4.9 | 93569_primary Th2_anti-CD28/anti-CD3 | 0.0 |
| Brain (fetal) | 0.0 | 93570_primary Tr1_anti-CD28/anti-CD3 | 0.0 |
| Brain (whole) | 0.4 | 93565_primary Th1_resting dy 4-6 in IL-2 | 0.0 |
| Brain (amygdala) | 0.8 | 93566_primary Th2_resting dy 4-6 in IL-2 | 0.0 |
| Brain (cerebellum) | 0.0 | 93567_primary Tr1_resting dy 4-6 in IL-2 | 0.0 |
| Brain (hippocampus) | 2.2 | 93351_CD45RA CD4 lymphocyte_anti- | 8.2 |
| | | CD28/anti-CD3 | |
| Brain (thalamus) | 0.0 | 93352_CD45RO CD4 lymphocyte_anti- | 0.0 |
| | | CD28/anti-CD3 | |
| Cerebral Cortex | 13.3 | 93251_CD8 Lymphocytes_anti-CD28/anti-CD3 | 0.0 |
| Spinal cord | 0.4 | 93353_chronic CD8 Lymphocytes 2ry_resting dy | 0.0 |
| | | 4-6 in IL-2 | |
| CNS ca. (glio/astro) | 100.0 | 93574_chronic CDS Lymphocytes 2ry_activated | 0.0 |
| U87-MG | | CD3/CD28 | |
| CNS ca. (glio/astro) | 6.8 | 93354_CD4_none | 0.0 |
| U-118-MG | | | |
| CNS ca. (astro) | 0.0 | 93252_Secondary Th1/Th2/Tr1_anti-CD95 | 0.0 |
| SW1783 | | | |
| CNS ca.* (neuro; met | 8.2 | 93103_LAK cells_resting | 0.4 |
| ) SK-N-AS | | | |
| CNS ca. (astro) | 1.3 | 93788_LAK cells_IL-2 | 0.0 |
| SF-539 | | | |
| CNS ca. (astro) | 2.6 | 93787_LAK cells_IL-2+IL-12 | 0.0 |
| SNB-75 | | | |
| CNS ca. (glio) | 0.0 | 93789_LAK cells_IL-2+IFN gamma | 0.5 |
| SNB-19 | | | |
| CNS ca. (glio) | 0.0 | 93790_LAK cells_IL-2+IL-18 | 0.0 |
| U251 | | | |
| CNS ca. (glio) | 0.0 | 93104_LAK cells_PMA/ionomycin and IL-18 | 0.0 |
| SF-295 | | | |
| Heart | 13.7 | 93578_NK Cells IL-2_resting | 0.0 |
| Skeletal Muscle (new | 10.6 | 93109_Mixed Lymphocyte Reaction_Two Way | 0.0 |
| lot*) | | MLR | |
| Bone marrow | 0.0 | 93110_Mixed Lymphocyte Reaction_Two Way | 0.0 |
| | | MLR | |
| Thymus | 0.0 | 93111_Mixed Lymphocyte Reaction_Two Way | 0.0 |
| | | MLR | |
| Spleen | 0.0 | 93112_Mononuclear Cells (PBMCs)_resting | 0.0 |
| Lymph node | 0.4 | 93113_Mononuclear Cells (PBMCs)_PWM | 0.0 |
| Colorectal | 14.7 | 93114_Mononuclear Cells (PBMCs)_PHA-L | 0.0 |
| Stomach | 6.0 | 93249_Ramos (B cell)_none | 0.0 |
| Small intestine | 2.9 | 93250_Ramos (B cell)_ionomycin | 0.0 |
| Colon ca. | 0.0 | 93349_B lymphocytes_PWM | 0.0 |
| SW480 | | | |
| Colon ca.* (SW480 | 0.0 | 93350_B lymphocytes_CD40L and IL-4 | 0.0 |
| met)SW620 | | | |
| Colon ca. | 0.0 | 92665_EOL-1 (Eosinophil)_dbcAMP | 0.0 |
| HT29 | | differentiated | |
| Colon ca. | 0.0 | 93248_EOL-1 | 0.0 |
| HCT-116 | | (Eosinophil)_dbcAMP/PMAionomycin | |
| Colon ca. | 0.0 | 93356_Dendritic Cells_none | 0.0 |
| CaCo-2 | | | |
| 83219 CC Well to | 9.7 | 93355_Dendritic Cells_LPS 100 ng/ml | 0.0 |
| Mod Diff(OD03866) | | | |
| Colon ca. | 4.1 | 93775_Dendritic Cells_anti-CD40 | 0.0 |
| HCC-2998 | | | |
| Gastric ca.* (liver | 0.0 | 93774_Monocytes_resting | 0.0 |
| met) NCI-N87 | | | |
| Bladder | 21.6 | 93776_Monocytes_LPS 50 ng/ml | 0.0 |
| Trachea | 0.8 | 93581_Macrophages_resting | 0.4 |
| Kidney | 0.0 | 93582_Macrophages_LPS 100 ng/ml | 0.0 |
| Kidney (fetal) | 33.4 | 93098_HUVEC (Endothelial)_none | 0.0 |
| Renal ca. | 0.0 | 93099_HUVEC (Endothelial)_starved | 0.8 |
| 786-0 | | | |
| Renal ca. | 0.0 | 93100_HUVEC (Endothelial)_IL-1b | 0.0 |
| A498 | | | |
| Renal ca. | 0.0 | 93779_HUVEC (Endothelial)_IFN gamma | 1.8 |
| RXF 393 | | | |
| Renal ca. | 0.0 | 93102_HUVEC (Endothelial)_TNF alpha +IFN | 1.2 |
| ACHN | | gamma | |
| Renal ca. | 0.0 | 93101_HUVEC (Endothelial)_TNF alpha +IL4 | 0.0 |
| UO-31 | | | |
| Renal ca. | 0.0 | 93781_HUVEC (Endothelial)_IL-11 | 1.8 |
| TK-10 | | | |
| Liver | 1.5 | 93583_Lung Microvascular Endothelial | 0.0 |
| | | Cells_none | |
| Liver (fetal) | 0.0 | 93584_Lung Microvascular Endothelial | 0.0 |
| | | Cells_TNFa (4 ng/ml) and IL1b (1 ng/ml) | |
| Liver ca. | 0.0 | 92662_Microvascular Dermal endothelium_none | 0.7 |
| (hepatoblast) HepG2 | | | |
| Lung | 5.1 | 92663_Microsvasular Dermal | 0.0 |
| | | endothelium_TNFa (4 ng/ml) and IL1b (1 ng/ml) | |
| Lung (fetal) | 1.9 | 93773_Bronchial epithelium_TNFa (4 ng/ml) | 2.2 |
| | | and IL1b (1ng/ml)** | |
| Lung ca. (small cell) | 0.0 | 93347_Small Airway Epithelium_none | 0.7 |
| LX-1 | | | |
| Lung ca. (small cell) | 0.0 | 93348_Small Airway Epithelium_TNFa (4 | 0.3 |
| NCI-H69 | | ng/ml) and IL1b (1 ng/ml) | |
| Lung ca. (s.cell var.) | 0.0 | 92668_Coronary Artery SMC_resting | 11.5 |
| SHP-77 | | | |
| Lung ca. (large | 0.0 | 92669_Coronary Artery SMC_TNFa (4 ng/ml) | 5.2 |
| cell)NCI-H460 | | and IL1b (1 ng/ml) | |
| Lung ca. (non-sm. | 0.0 | 93107_astrocytes_resting | 0.7 |
| cell) A549 | | | |
| Lung ca. (non-s.cell) | 0.0 | 93108_astrocytes_TNFa (4 ng/ml) and IL1b (1 | 0.3 |
| NCI-H23 | | ng/ml) | |
| Lung ca (non-s.cell) | 0.8 | 92666_KU-812 (Basophil)_resting | 0.0 |
| HOP-62 | | | |
| Lung ca. (non-s.cl) | 0.0 | 92667_KU-812 (Basophil)_PMA/ionoycin | 0.0 |
| NCI-H522 | | | |
| Lung ca. (squam.) | 0.2 | 93579_CCD1106 (Keratinocytes)_none | 0.0 |
| SW 900 | | | |
| Lung ca. (squam.) | 0.0 | 93580_CCD1106 (Keratinocytes)_TNFa and | 0.0 |
| NCI-H596 | | IFNg ** | |
| Mammary gland | 1.3 | 93791_Liver Cirrhosis | 11.3 |
| Breast ca* (pl. | 20.3 | 93792_Lupus Kidney | 12.9 |
| effusion) MCF-7 | | | |
| Breast ca.* (pl.ef) | 0.0 | 93577_NCI-H292 | 0.0 |
| MDA-MB-231 | | | |
| Breast ca.* (pl. | 0.2 | 93358_NCI-H292_IL-4 | 0.0 |
| effusion) T47D | | | |
| Breast ca. | 16.3 | 93360_NCI-H292_IL-9 | 0.0 |
| BT-549 | | | |
| Breast ca. | 0.0 | 93359_NCI-H292_IL-13 | 0.0 |
| MDA-N | | | |
| Ovary | 1.9 | 93357_NCI-H292_IFN gamma | 0.0 |
| Ovarian ca. | 4.8 | 93777_HPAEC_- | 1.6 |
| OVCAR-3 | | | |
| Ovarian ca. | 0.0 | 93778_HPAEC_IL-1 beta/TNA alpha | 0.0 |
| OVCAR-4 | | | |
| Ovarian ca. | 9.0 | 93254_Normal Human Lung Fibroblast_none | 42.9 |
| OVCAR-5 | | | |
| Ovarian ca. | 13.3 | 93253_Normal Human Lung Fibroblast_TNFa (4 | 14.9 |
| OVCAR-8 | | ng/ml) and IL-1b (1 ng/ml) | |
| Ovarian ca. | 0.0 | 93257_Normal Human Lung Fibroblast_IL-4 | 39.0 |
| IGROV-1 | | | |
| Ovarian ca. * (ascites) | 0.0 | 93256_Normal Human Lung Fibroblast_IL-9 | 29.5 |
| SK-OV-3 | | | |
| Uterus | 6.4 | 93255_Normal Human Lung Fibroblast_IL-13 | 96.6 |
| Placenta | 2.8 | 93258_Normal Human Lung Fibroblast_IFN | 40.3 |
| | | gamma | |
| Prostate | 3.1 | 93106_Dermal Fibroblasts CCD1070_resting | 25.9 |
| Prostate ca.* (bone | 32.1 | 93361_Dermal Fibroblasts CCD1070_TNF alpha | 22.5 |
| met)PC-3 | | 4 ng/ml | |
| Testis | 0.4 | 93105_Dermal Fibroblasts CCD 1070_IL-1 beta | 29.5 |
| | | 1 ng/ml | |
| Melanoma | 13.1 | 93772_dermal fibroblast_IFN gamma | 17.9 |
| Hs688(A).T | | | |
| Melanoma* (met) | 7.4 | 93771_dermal fibroblast_IL-4 | 81.8 |
| Hs688(B).T | | | |
| Melanoma | 0.5 | 93259_IBD Colitis 1** | 33.2 |
| UACC-62 | | | |
| Melanoma | 0.0 | 93260_IBD Colitis 2 | 7.7 |
| M14 | | | |
| Melanoma | 3.8 | 93261_IBD Crohns | 4.2 |
| LOX IMVI | | | |
| Melanoma* (met) | 6.4 | 735010_Colon_normal | 21.9 |
| SK-MEL-5 | | | |
| Adipose | 47.3 | 735019_Lung_none | 34.6 |
| | | 64028-1_Thymus_none | 100.0 |
| | | 64030-1_Kidney_none | 14.8 |

As is shown in Table 16H (Panel 1.2), POLY9 shows variable expression in a number of samples across different cell types. Of interest is the difference in expression between the fetal kidney and adult kidney samples. The data reveal that POLY9 is expressed higher in the fetal kidney versus adult kidney, which suggests that this gene may play a role in organogenesis. This may indicate that this gene could be used as a therapy driving kidney regeneration.

As is shown in Table 16H (Panel 4D), POLY9 is highly expressed in the thymus, lung, colon and kidney and in fibroblasts. Antibodies to a POLY9 polypeptide may serve as a marker for fibroblasts. This protein may also be important in the treatment of psoriasis.

E. POLY10.

Quantitative expression of POLY10 was assessed using the primer-probe sets Ag1701/Ag1159 (identical sequences), described in Table 161. Results of the RTQ-PCR runs are shown in Tables 16J and 16K.

TABLE 16I
Probe Name: Ag1701/Ag1159

| | | | | | Start |
|---|---|---|---|---|---|
| Primers | Sequences | | TM | Length | Position |
| Forward | 5′-GGACAGGGTGACTAGGTCATCT-3′ | (SEQ ID NO.:60) | 59.5 | 22 | 50 |
| Probe | FAM-5′-CAAACATGCTGTATGTCAATGG | (SEQ ID NO.:61) | 67.9 | 26 | 73 |
| | CACA-3′-TAMRA | | | | |
| Reverse | 5′-GCTGAGGACCAGTTGTATGG-3′ | (SEQ ID NO.:62) | 58.2 | 20 | 115 |

TABLE 16J
Panels 1.3D and 2D

| PANEL 1.3D | | PANEL 2D | |
|---|---|---|---|
| | Relative | | Relative |
| Tissue | Expression(%) | Tissue | Expression(%) |
| Name | 1.3dtm3247t_ag1159 | Name | 2dtm3248t_1159 |
| Liver adenocarcinoma | 19.10 | Normal Colon GENPAK | 66.00 |
| | | 061003 | |
| Pancreas | 2.10 | 83219 CC Well to Mod Diff | 11.50 |
| | | (ODO3866) | |
| Pancreatic ca. CAPAN 2 | 9.60 | 83220 CC NAT | 12.10 |
| | | (ODO3866) | |
| Adrenal gland | 8.10 | 83221 CC Gr.2 | 15.30 |
| | | rectosigmoid (ODO3868) | |
| Thyroid | 15.30 | 83222 CC NAT | 5.00 |
| | | (ODO3868) | |
| Salivary gland | 3.30 | 83235 CC Mod Diff | 21.00 |
| | | (ODO3920) | |
| Pituitary gland | 4.90 | 83236 CC NAT | 19.60 |
| | | (ODO3920) | |
| Brain (fetal) | 10.70 | 83237 CC Gr.2 ascend | 28.10 |
| | | colon (ODO3921) | |
| Brain (whole) | 12.40 | 83238 CC NAT | 9.20 |
| | | (ODO3921) | |
| Brain (amygdala) | 7.90 | 83241 CC from Partial | 57.00 |
| | | Hepatectomy (ODO4309) | |
| Brain (cerebellum) | 5.30 | 83242 Liver NAT | 100.00 |
| | | (ODO4309) | |
| Brain (hippocampus) | 23.70 | 87472 Colon mets to lung | 12.40 |
| | | (OD04451-01) | |
| Brain (substantia nigra) | 2.80 | 87473 Lung NAT | 32.10 |
| | | (OD04451-02) | |
| Brain (thalamus) | 3.80 | Normal Prostate Clontech | 18.60 |
| | | A+ 6546-1 | |
| Cerebral Cortex | 100.00 | 84140 Prostate Cancer | 34.90 |
| | | (OD04410) | |
| Spinal cord | 4.20 | 84141 Prostate NAT | 53.20 |
| (OD04410) | | | |
| CNS ca. (glio/astro) U87-MG | 18.90 | 87073 Prostate Cancer | 37.40 |
| | | (OD04720-01) | |
| CNS ca. (glio/astro) U-118-MG | 47.00 | 87074 Prostate NAT | 33.20 |
| | | (OD04720-02) | |
| CNS ca. (astro) SW1783 | 28.30 | Normal Lung GENPAK | 60.30 |
| | | 061010 | |
| CNS ca* (neuro; met) SK-N-AS | 10.00 | 83239 Lung Met to Muscle | 25.70 |
| | | (ODO4286) | |
| CNS ca. (astro) SF-539 | 21.90 | 83240 Muscle NAT | 27.00 |
| | | (ODO4286) | |
| CNS ca. (astro) SNB-75 | 34.20 | 84136 Lung Malignant | 30.40 |
| | | Cancer (OD03126) | |
| CNS Ca. (glio) SNB-19 | 36.90 | 84137 Lung NAT | 49.70 |
| | | (OD03126) | |
| CNS Ca. (glio) U251 | 13.70 | 84871 Lung Cancer | 34.60 |
| | | (OD04404) | |
| CNS Ca. (glio) SF-295 | 35.40 | 84872 Lung NAT | 24.30 |
| (OD04404) | | | |
| Heart (fetal) | 9.30 | 84875 Lung Cancer | 11.70 |
| | | (OD04565) | |
| Heart | 4.40 | 84876 Lung NAT | 17.40 |
| | | (OD04565) | |
| Fetal Skeletal | 95.30 | 85950 Lung Cancer | 35.40 |
| | | (OD04237-01) | |
| Skeletal muscle | 1.70 | 85970 Lung NAT | 42.00 |
| (OD04237-02) | | | |
| Bone marrow | 1.50 | 83255 Ocular Mel Met to | 4.40 |
| | | Liver (ODO4310) | |
| Thymus | 3.50 | 83256 Liver NAT | 38.40 |
| | | (ODO4310) | |
| Spleen | 26.40 | 84139 Melanoma Mets to | 11.20 |
| Lung (OD04321) | | | |
| Lymph node | 5.30 | 84138 Lung NAT | 41.80 |
| (OD04321) | | | |
| Colorectal | 18.70 | Normal Kidney GENPAK | 45.70 |
| | | 061008 | |
| Stomach | 9.50 | 83786 Kidney Ca, Nuclear | 27.70 |
| | | grade 2 (OD04338) | |
| Small intestine | 10.20 | 83787 Kidney NAT | 42.00 |
| | | (OD04338) | |
| Colon ca. SW480 | 17.40 | 83788 Kidney Ca Nuclear | 24.50 |
| | | grade 1/2 (OD04339) | |
| Colon ca.* (SW480 met)SW620 | 4.70 | 83789 Kidney NAT | 27.70 |
| | | (OD04339) | |
| Colon ca. HT29 | 3.50 | 83790 Kidney Ca, Clear cell | 28.70 |
| | | type (OD04340) | |
| Colon ca. HCT-116 | 4.10 | 83791 Kidney NAT | 49.00 |
| | | (OD04340) | |
| Colon ca. CaCo-2 | 50.00 | 83792 Kidney Ca, Nuclear | 25.00 |
| | | grade 3 (OD04348) | |
| 83219 CC Well to Mod Diff | 11.20 | 83793 Kidney NAT | 33.00 |
| (ODO3866) | | (OD04348) | |
| Colon ca. HCC-2998 | 3.50 | 87474 Kidney Cancer | 6.60 |
| | | (OD04622-01) | |
| Gastric ca.* (liver met) NCI-N87 | 9.00 | 87475 Kidney NAT | 4.60 |
| | | (OD04622-03) | |
| Bladder | 13.10 | 85973 Kidney Cancer | 7.20 |
| | | (OD04450-01) | |
| Trachea | 10.60 | 85974 Kidney NAT | 26.80 |
| | | (OD04450-03) | |
| Kidney | 2.00 | Kidney Cancer Clontech | 5.10 |
| | | 8120607 | |
| Kidney (fetal) | 9.90 | Kidney NAT Clontech | 4.20 |
| | | 8120608 | |
| Renal ca. 786-0 | 12.30 | Kidney Cancer Clontech | 8.20 |
| | | 8120613 | |
| Renal ca. A498 | 16.40 | Kidney NAT Clontech | 7.90 |
| | | 8120614 | |
| Renal ca. RXF 393 | 8.80 | Kidney Cancer Clontech | 21.60 |
| | | 9010320 | |
| Renal ca. ACHN | 8.30 | Kidney NAT Clontech | 19.20 |
| | | 9010321 | |
| Renal ca. UO-31 | 7.00 | Normal Uterus GENPAK | 7.90 |
| | | 061018 | |
| Renal ca. TK-10 | 10.40 | Uterus Cancer GENPAK | 17.20 |
| | | 064011 | |
| Liver | 15.50 | Normal Thyroid Clontech | 35.10 |
| | | A+ 6570-1 | |
| Liver (fetal) | 17.90 | Thyroid Cancer GENPAK | 72.20 |
| | | | 064010 |
| Liver ca. (hepatoblast) HepG2 | 6.90 | Thyroid Cancer | 20.90 |
| | | INVITROGEN A302152 | |
| Lung | 12.90 | Thyroid NAT | 57.80 |
| | | INVITROGEN A302153 | |
| Lung (fetal) | 19.80 | Normal Breast GENPAK | 47.60 |
| | | 061019 | |
| Lung ca. (small cell) LX-1 | 6.50 | 84877 Breast Cancer | 22.10 |
| | | (OD04566) | |
| Lung ca. (small cell) NCI-H69 | 25.90 | 85975 Breast Cancer | 59.90 |
| | | (OD04590-01) | |
| Lung ca. (s.cell var.) SHP-77 | 10.70 | 85976 Breast Cancer Mets | 58.60 |
| | | (OD04590-03) | |
| Lung ca. (large cell)NCI-H460 | 12.20 | 87070 Breast Cancer | 32.80 |
| | | Metastasis (OD04655-05) | |
| Lung ca. (non-sm. cell) A549 | 11.40 | GENPAK Breast Cancer | 21.90 |
| | | 064006 | |
| Lung ca. (non-s.cell) NCI-H23 | 14.00 | Breast Cancer Res. Gen. | 27.20 |
| | | 1024 | |
| Lung ca (non-s.cell) HOP-62 | 27.40 | Breast Cancer Clontech | 30.40 |
| | | 9100266 | |
| Lung ca. (non-s.d) NCI-H522 | 28.50 | Breast NAT Clontech | 19.50 |
| | | 9100265 | |
| Lung ca. (squam.) SW 900 | 16.00 | Breast Cancer | 49.00 |
| | | INVITROGEN A209073 | |
| Lung ca. (squam.) NCI-H596 | 3.00 | Breast NAT INVITROGEN | 27.20 |
| | | A2090734 | |
| Mammary gland | 16.20 | Normal Liver GENPAK | 94.60 |
| | | 061009 | |
| Breast ca.* (pl. effusion) MCF-7 | 36.30 | Liver Cancer GENPAK | 66.00 |
| | | 064003 | |
| Breast ca.* (pl.ef) MDA-MB-231 | 51.80 | Liver Cancer Research | 80.10 |
| | | Genetics RNA 1025 | |
| Breast ca.* (pl. effusion) T47D | 20.60 | Liver Cancer Research | 19.60 |
| | | Genetics RNA 1026 | |
| Breast ca. BT-549 | 16.70 | Paired Liver Cancer Tissue | 97.30 |
| | | Research Genetics RNA | |
| | | 6004-T | |
| Breast ca. MDA-N | 12.70 | Paired Liver Tissue | 15.90 |
| | | Research Genetics RNA | |
| | | 6004-N | |
| Ovary | 17.70 | Paired Liver Cancer Tissue | 26.80 |
| | | Research Genetics RNA | |
| | | 6005-T | |
| Ovarian ca. OVCAR-3 | 3.10 | Paired Liver Tissue | 10.70 |
| | | Research Genetics RNA | |
| | | 6005-N | |
| Ovarian ca. OVCAR-4 | 2.20 | Normal Bladder GENPAK | 50.30 |
| | | 061001 | |
| Ovarian ca. OVCAR-5 | 12.50 | Bladder Cancer Research | 6.40 |
| | | Genetics RNA 1023 | |
| Ovarian ca. OVCAR-8 | 20.70 | Bladder Cancer | 11.70 |
| | | INVITROGEN A302173 | |
| Ovarian ca. IGROV-1 | 10.70 | 87071 Bladder Cancer | 26.10 |
| (OD04718-01) | | | |
| Ovarian ca* (ascites) SK-OV-3 | 22.50 | 87072 Bladder Normal | 20.30 |
| | | Adjacent (OD04718-03) | |
| Uterus | 6.20 | Normal Ovary Res. Gen. | 4.90 |
| Placenta | 7.30 | Ovarian Cancer GENPAK | 27.40 |
| | | 064008 | |
| Prostate | 6.60 | 87492 Ovary Cancer | 41.80 |
| | | (OD04768-07) | |
| Prostate ca.* (bone met)PC-3 | 7.60 | 87493 Ovary NAT | 13.70 |
| | | (OD04768-08) | |
| Testis | 12.60 | Normal Stomach GENPAK | 21.00 |
| | | 061017 | |
| Melanoma Hs688(A).T | 34.20 | Gastric Cancer Clontech | 8.20 |
| | | 9060358 | |
| Melanoma* (met) Hs688(B).T | 54.30 | NAT Stomach Clontech | 14.50 |
| | | 9060359 | |
| Melanoma UACC-62 | 3.30 | Gastric Cancer Clontech | 17.80 |
| | | 9060395 | |
| Melanoma M14 | 4.00 | NAT Stomach Clontech | 11.60 |
| | | 9060394 | |
| Melanoma LOX IMVI | 5.80 | Gastric Cancer Clontech | 36.10 |
| | | 9060397 | |
| Melanoma* (met) SK-MEL-S | 8.80 | NAT Stomach Clontech | 8.50 |
| | | 9060396 | |
| Adipose | 9.20 | Gastric Cancer GENPAK | 60.70 |
| | | 064005 | |

TABLE 16K
Panel 4D: Ag1159

| | Relative Expression (%) | | |
|---|---|---|---|
| Tissue Name | 4Dtm1850t | 4Dtm1915t | 4dtm3249t |
| 93768_Secondary Th1_anti-CD28/anti-CD3 | 0.9 | 0.6 | 1.5 |
| 93769_Secondary Th2_anti-CD28/anti-CD3 | 0.4 | 0.3 | 1.2 |
| 93770_Secondary Tr1_anti-CD28/anti-CD3 | 0.7 | 0.3 | 1.0 |
| 93573_Secondary Th1_resting day 4-6 in IL-2 | 0.2 | 0.0 | 0.2 |
| 93572_Secondary Th2_resting day 4-6 in IL-2 | 0.0 | 0.2 | 0.4 |
| 93571_Secondary Tr1_resting day 4-6 in IL-2 | 0.0 | 0.0 | 0.2 |
| 93568_primary Th1_anti-CD28/anti-CD3 | 3.1 | 2.5 | 6.2 |
| 93569_primary Th2_anti-CD28/anti-CD3 | 3.0 | 2.2 | 5.6 |
| 93570_primary Tr1_anti-CD28/anti-CD3 | 4.6 | 4.2 | 9.7 |
| 93565_primary Th1_resting dy 4-6 in IL-2 | 1.4 | 1.1 | 4.6 |
| 93566_primary Th2_resting dy 4-6 in IL-2 | 0.5 | 0.4 | 1.8 |
| 93567_primary Tr1_resting dy 4-6 in IL-2 | 0.7 | 0.8 | 1.2 |
| 93351_CD45RA CD4 lymphocyte_anti-CD28/anti- | 5.5 | 4.1 | 15.4 |
| CD3 | | | |
| 93352_CD45RO CD4 lymphocyte_anti-CD28/anti- | 2.1 | 2.4 | 6.1 |
| CD3 | | | |
| 93251_CD8 Lymphocytes_anti-CD28/anti-CD3 | 0.4 | 0.3 | 1.0 |
| 93353_chronic CD8 Lymphocytes 2ry_resting dy 4-6 | | | |
| in IL-2 | 2.8 | 2.2 | 4.9 |
| 93574_chronic CD8 Lymphocytes 2ry_activated | | | |
| CD3/CD28 | 0.1 | 0.2 | 0.4 |
| 93354_CD4_none | 0.2 | 0.4 | 0.6 |
| 93252_Secondary Th1/Th2/Tr1_anti-CD95 CH11 | 0.5 | 0.4 | 1.1 |
| 93103_LAK cells_resting | 4.5 | 5.0 | 9.6 |
| 93788_LAK cells_IL-2 | 0.0 | 0.2 | 0.6 |
| 93787_LAK cells_IL-2+IL-12 | 0.4 | 0.3 | 1.1 |
| 93789_LAK cells_IL-2+IFN gamma | 0.5 | 0.5 | 1.6 |
| 93790_LAK cells_IL-2+IL-18 | 0.4 | 0.3 | 1.6 |
| 93104_LAKcells_PMA/ionomycin and IL-18 | 11.2 | 11.3 | 36.3 |
| 93578_NK Cells IL-2_resting | 0.0 | 0.0 | 0.1 |
| 93109_Mixed Lymphocyte Reaction_Two Way | | | |
| MLR | 1.6 | 1.3 | 4.6 |
| 93110_Mixed Lymphocyte Reaction_Two Way | | | |
| MLR | 0.8 | 0.8 | 1.5 |
| 93111_Mixed Lymphocyte Reaction_Two Way | | | |
| MLR | 0.2 | 0.2 | 0.5 |
| 93112_Mononuclear Cells (PBMCs)_resting | 0.4 | 0.5 | 1.7 |
| 93113_Mononuclear Cells (PBMCs)_PWM | 4.7 | 4.2 | 9.2 |
| 93114_Mononuclear Cells (PBMCs)_PHA-L | 5.1 | 4.1 | 7.9 |
| 93249_Ramos (B cell)_none | 0.2 | 0.1 | 0.2 |
| 93250_Ramos (B cell)_ionomycin | 0.8 | 1.2 | 3.0 |
| 93349_B lymphocytes_PWM | 3.3 | 2.5 | 11.3 |
| 93350_B lymphocytes_CD40L and IL-4 | 2.2 | 1.7 | 6.6 |
| 92665_EOL-1 (Eosinophil)_dbcAMP differentiated | 0.2 | 0.1 | 0.7 |
| 93248_EOL-1 | | | |
| (Eosinophil)dbcAMP/PMAionomycin | 3.4 | 3.2 | 9.4 |
| 93356_Dendritic Cells_none | 12.4 | 10.4 | 33.2 |
| 93355_Dendritic Cells_LPS 100 ng/ml | 9.3 | 8.1 | 24.1 |
| 93775_Dendritic Cells_anti-CD40 | 19.9 | 17.0 | 59.0 |
| 93774_Monocytes_resting | 1.6 | 1.8 | 4.6 |
| 93776_Monocytes_LPS 50 ng/ml | 5.9 | 4.7 | 9.3 |
| 93581_Macrophages_resting | 14.7 | 11.9 | 28.7 |
| 93582_Macrophages_LPS 100 ng/ml | 6.9 | 5.2 | 9.5 |
| 93098_HUVEC (Endothelial)_none | 7.7 | 8.3 | 23.3 |
| 93099_HUVEC (Endothelial)_starved | 15.1 | 11.9 | 52.1 |
| 93100_HUVEC (Endothelial)_IL-1b | 21.9 | 18.6 | 58.2 |
| 93779_HUVEC (Endothelial)_IFN gamma | 9.7 | 8.8 | 31.9 |
| 93102_HUVEC (Endothelial)_TNF alpha+IFN | | | |
| gamma | 7.2 | 6.7 | 33.4 |
| 93101_HUVEC (Endothelial)_TNF alpha+IL4 | 11.9 | 9.5 | 37.4 |
| 93781_HUVEC (Endothelial)_IL-11 | 8.0 | 5.5 | 18.4 |
| 93583_Lung Microvascular Endothelial Cells_none | 11.7 | 9.9 | 33.0 |
| 93584_Lung Microvascular Endothelial Cells_TNFa | | | |
| (4 ng/ml) and IL1b (1 ng/ml) | 12.1 | 8.8 | 43.8 |
| 92662_Microvascular Dermal endothelium_none | 23.7 | 20.2 | 41.5 |
| 92663_Microsvasular Dermal endothelium_TNFa (4 | | | |
| ng/ml) and IL1b (1 ng/ml) | 23.8 | 24.5 | 52.1 |
| 93773_Bronchial epithelium_TNFa (4 ng/ml) and | | | |
| IL1b (1 ng/ml)** | 14.0 | 15.0 | 6.0 |
| 93347_Small Airway Epithelium_none | 2.8 | 1.8 | 9.5 |
| 93348_Small Airway Epithelium_TNFa (4 ng/ml) | | | |
| and IL1b (1 ng/ml) | 35.6 | 36.3 | 100.0 |
| 92668_Coronery Artery SMC_resting | 7.9 | 5.8 | 27.7 |
| 92669_Coronery Artery SMC_TNFa (4 ng/ml) and | | | |
| IL1b (1 ng/ml) | 4.9 | 3.6 | 15.3 |
| 93107_astrocytes_resting | 18.9 | 17.8 | 85.9 |
| 93108_astrocytes_TNFa (4 ng/ml) and IL1b (1 | | | |
| ng/ml) | 24.5 | 17.6 | 60.7 |
| 92666_KU-812 (Basophil)_resting | 0.2 | 0.2 | 0.8 |
| 92667_KU-812 (Basophil)_PMA/ionoycin | 2.0 | 1.3 | 4.5 |
| 93579_CCD1106 (Keratinocytes)_none | 5.1 | 4.9 | 16.8 |
| 93580_CCD1106 (Keratinocytes)_TNFa and IFNg | | | |
| ** | 100.0 | 100.0 | 8.1 |
| 93791_Liver Cirrhosis | 3.5 | 4.2 | 8.2 |
| 93792_Lupus Kidney | 7.3 | 6.7 | 8.0 |
| 93577_NCI-H292 | 13.1 | 8.8 | 29.9 |
| 93358_NCI-H292_IL-4 | 16.5 | 15.9 | 60.3 |
| 93360_NCI-H292_IL-9 | 13.5 | 11.8 | 47.3 |
| 93359_NCI-H292_IL-13 | 11.3 | 9.5 | 36.9 |
| 93357_NCI-H292_IFN gamma | 7.9 | 7.2 | 19.5 |
| 93777_HPAEC_- | 13.1 | 10.8 | 30.1 |
| 93778_HPAEC_IL-1 beta/TNA alpha | 26.1 | 21.5 | 76.8 |
| 93254_Normal Human Lung Fibroblast_none | 7.4 | 6.3 | 19.5 |
| 93253_Normal Human Lung Fibroblast_TNFa (4 | | | |
| ng/ml) and IL-1b (1 ng/ml) | 3.0 | 2.6 | 9.7 |
| 93257_Normal Human Lung Fibroblast_IL-4 | 6.8 | 7.5 | 19.1 |
| 93256_Normal Human Lung Fibroblast_IL-9 | 10.1 | 8.8 | 27.2 |
| 93255_Normal Human Lung Fibroblast_IL-13 | 0.0 | 9.0 | 16.8 |
| 93258_Normal Human Lung Fibroblast_IFN gamma | 10.5 | 9.1 | 23.0 |
| 93106_Dermal Fibroblasts CCD1070_resting | 18.9 | 15.7 | 54.3 |
| 93361_Dermal Fibroblasts CCD1070_TNF alpha 4 | | | |
| ng/ml | 36.9 | 28.7 | 82.4 |
| 93105 Dermal Fibroblasts CCD1070 IL-1 beta 1 | 13.2 | 10.6 | 37.4 |
| ng/ml | | | |
| 93772_dermal fibroblast_IFN gamma | 6.9 | 5.8 | 19.6 |
| 93771_dermal fibroblast_IL-4 | 12.2 | 12.1 | 33.7 |
| 93259_IBD Colitis 1** | 4.4 | 3.0 | 1.8 |
| 93260_IBD Colitis 2 | 0.8 | 0.5 | 2.3 |
| 93261_IBD Crohns | 2.0 | 1.9 | 7.7 |
| 735010_Colon_normal | 7.6 | 7.9 | 36.3 |
| 735019_Lung_none | 15.7 | 13.0 | 39.5 |
| 64028-1_Thymus_none | 24.0 | 18.0 | 33.7 |
| 64030-1_Kidney_none | 6.0 | 4.5 | 20.4 |

As is shown in Table 16J (Panel 1.3D), POLY10 shows a general ubiquitous expression across the samples. However, the highest expression is in the sample of cerebral cortex and the second highest expression level is in fetal skeletal muscle. The difference in expression between fetal skeletal muscle and adult skeletal muscle is very striking indicating that this gene may play a role in musculogenesis and possibly be used as a therapeutic for muscle regeneration.

As is shown in Table 16J (Panel 2D), POLY10 is expressed in a variety of cancer tissues, with highest levels being in liver cancers and lower levels in thyroid cancer and breast cancer.

As is shown in Table 16K (Panel 4D), POLY10 is broadly expressed at low levels in fibroblasts and in the endothelium which is reflected in expression in tissues which have this type of cell such as the colon. Keratinocytes and small airway epithelium highly upregulate the expression of this molecule after treatment with TNF-alpha and IL-1 beta. Antagonistic therapeutics to the protein encoded for by this transcript could inhibit or block inflammation in psoriasis, delayed type hypersensitivity, asthma, allergy, and emphysema.

F. POLY11.

Quantitative expression of POLY11 was assessed using the primer-probe set Ag 1338, described in Table 16L.

TABLE 16L
Probe Name: Ag1338

| | | | | | Start |
|---|---|---|---|---|---|
| Primers | Sequences | | TM | Length | Position |
| Forward | 5′-CCAGCGTTTCACGAGTCTT-3′ | (SEQ ID NO.:63) | 59 | 19 | 13 |
| Probe | TET-5′-CAAGCCTTCAGGCTTTCTTTAA | (SEQ ID NO.:64) | 64.8 | 26 | 32 |
| | TCAA-3′-TAMRA | | | | |
| Reverse | 5′-TCACTTCTGACAAGTTGGGTTT-3′ | (SEQ ID NO.:65) | 58.7 | 22 | 68 |

Expression of POLY11 was low to undetectable (Ct>35) on panels 1.2 and 4D.

G. POLY12.

Quantitative expression of POLY12 was assessed using the primer-probe set Ag1160, described in Table 16M. Results of the RTQ-PCR runs are shown in Tables 16N and 160.

TABLE 16M
Probe Name: Ag1160

| | | | | | Start |
|---|---|---|---|---|---|
| Primers | Sequences | | TM | Length | Position |
| Forward | 5′-ATACATGGAGGTGGCTAAAACC-3′ | (SEQ ID NO.:66) | 59.3 | 22 | 1187 |
| Probe | TET-5′-TCATAATCACGGAATTCACGCT | (SEQ ID NO.:67) | 68 | 29 | 1223 |
| | ACTACCA-3′-TAMRA | | | | |
| Reverse | 5′-ACACTAGCAAATTCAGGCTGAA-3′ | (SEQ ID NO.:68) | 59 | 22 | 1252 |

TABLE 16N
Panel 1.2 (Run 1 and Run 2)

| | Relative Expression(%) | |
|---|---|---|
| Tissue Name | 1.2tm1384t_ag1160 | 1.2tm1443t_ag1160 |
| Endothelial cells | 14.8 | 12.9 |
| Endothelial cells (treated) | 12.9 | 13.1 |
| Pancreas | 5.0 | 2.7 |
| Pancreatic ca. CAPAN 2 | 29.1 | 23.7 |
| Adrenal Gland (new lot*) | 74.2 | 95.9 |
| Thyroid | 10.4 | 3.1 |
| Salivary gland | 28.9 | 35.6 |
| Pituitary gland | 8.2 | 5.4 |
| Brain (fetal) | 2.5 | 4.8 |
| Brain (whole) | 26.1 | 15.9 |
| Brain (amygdala) | 13.6 | 21.0 |
| Brain (cerebellum) | 15.8 | 23.7 |
| Brain (hippocampus) | 21.5 | 51.8 |
| Brain (thalamus) | 9.7 | 7.5 |
| Cerebral Cortex | 42.0 | 42.9 |
| Spinal cord | 13.4 | 13.1 |
| CNS ca. (glio/astro) U87-MG | 33.9 | 6.5 |
| CNS ca. (glio/astro) U-118-MG | 13.0 | 14.1 |
| CNS ca. (astro) SW1783 | 3.2 | 3.1 |
| CNS ca.* (neuro; met) SK-N-AS | 18.9 | 10.7 |
| CNS ca. (astro) SF-539 | 0.9 | 0.6 |
| CNS ca. (astro) SNB-75 | 0.4 | 0.4 |
| CNS ca. (glio) SNB-19 | 1.3 | 0.9 |
| CNS ca. (glio) U251 | 0.6 | 0.4 |
| CNS ca. (glio) SF-295 | 3.1 | 4.2 |
| Heart | 32.5 | 17.9 |
| Skeletal Muscle (new lot*) | 6.6 | 3.4 |
| Bone marrow | 6.8 | 6.6 |
| Thymus | 2.6 | 2.0 |
| Spleen | 7.7 | 7.2 |
| Lymph node | 5.4 | 6.3 |
| Colorectal | 26.8 | 13.5 |
| Stomach | 17.7 | 11.7 |
| Small intestine | 12.1 | 7.0 |
| Colon ca. SW480 | 1.9 | 0.5 |
| Colon ca.* (SW480 met)SW620 | 6.3 | 5.0 |
| Colon ca. HT29 | 6.4 | 1.4 |
| Colon ca. HCT-116 | 7.3 | 2.5 |
| Colon ca. CaCo-2 | 24.3 | 8.4 |
| 83219 CC Well to Mod Diff (ODO3866) | 13.3 | 11.0 |
| Colon ca. HCC-2998 | 100.0 | 37.1 |
| Gastric ca.* (liver met) NCI-N87 | 45.7 | 16.2 |
| Bladder | 45.1 | 45.4 |
| Trachea | 7.2 | 6.3 |
| Kidney | 25.3 | 12.1 |
| Kidney (fetal) | 24.1 | 13.7 |
| Renal ca. 786-0 | 9.7 | 3.6 |
| Renal ca. A498 | 50.7 | 50.0 |
| Renal ca. RXF 393 | 6.0 | 6.7 |
| Renal ca. ACHN | 23.5 | 18.3 |
| Renal ca. UO-31 | 5.5 | 2.1 |
| Renal ca. TK-10 | 23.5 | 14.2 |
| Liver | 33.9 | 42.9 |
| Liver (fetal) | 40.6 | 35.1 |
| Liver ca. (hepatoblast) HepG2 | 43.5 | 25.3 |
| Lung | 4.8 | 4.2 |
| Lung (fetal) | 3.8 | 3.8 |
| Lung ca. (small cell) LX-1 | 11.7 | 10.2 |
| Lung ca. (small cell) NCI-H69 | 11.1 | 12.4 |
| Lung ca. (s.cell var.) SHP-77 | 1.8 | 0.7 |
| Lung ca (large cell)NCI-H460 | 53.2 | 70.7 |
| Lung ca. (non-sm. cell) A549 | 17.4 | 10.9 |
| Lung ca. (non-s.cell) NCI-H23 | 3.9 | 4.1 |
| Lung ca (non-s.cell) HOP-62 | 16.5 | 12.5 |
| Lung ca. (non-s.cl) NCI-H522 | 6.6 | 6.9 |
| Lung ca. (squam.) SW 900 | 10.0 | 4.8 |
| Lung ca. (squam.) NCI-H596 | 10.2 | 9.3 |
| Mammary gland | 11.1 | 6.6 |
| Breast ca.* (pl. effusion) MCF-7 | 31.4 | 8.8 |
| Breast ca.* (pl.ef) MDA-MB-231 | 11.2 | 4.4 |
| Breast ca.* (pl. effusion) T47D | 5.7 | 2.4 |
| Breast ca BT-549 | 12.6 | 9.3 |
| Breast ca. MDA-N | 9.0 | 3.1 |
| Ovary | 10.7 | 10.0 |
| Ovarian ca. OVCAR-3 | 15.1 | 10.4 |
| Ovarian ca. OVCAR-4 | 5.6 | 7.1 |
| Ovarian ca. OVCAR-5 | 34.4 | 20.2 |
| Ovarian ca. OVCAR-8 | 17.9 | 2.9 |
| Ovarian ca. IGROV-1 | 26.4 | 13.8 |
| Ovarian ca. (ascites) SK-OV-3 | 50.0 | 51.4 |
| Uterus | 9.3 | 5.2 |
| Placenta | 38.7 | 25.5 |
| Prostate | 12.2 | 8.9 |
| Prostate ca.* (bone met)PC-3 | 13.4 | 30.8 |
| Testis | 27.7 | 19.2 |
| Melanoma Hs688(A).T | 10.4 | 7.8 |
| Melanoma* (met) Hs688(B).T | 9.3 | 6.7 |
| Melanoma UACC-62 | 9.9 | 9.3 |
| Melanoma M14 | 7.5 | 12.1 |
| Melanoma LOX IMVI | 5.3 | 3.3 |
| Melanoma* (met) SK-MEL-5 | 12.8 | 16.5 |
| Adipose | 82.9 | 100.0 |

TABLE 16O
Panels 2D and 4D

| PANEL 2D | | PANEL 4D | |
|---|---|---|---|
| | Relative | | Relative |
| | Expression (%) | | Expression (%) |
| | 2dx4tm5022t_a | | 4dtm2014t_ag |
| Tissue Name | g1160_b2 | Tissue Name | 1160 |
| Normal Colon | 63.5 | 93768_Secondary Th1_anti-CD28/anti- | 14.0 |
| GENPAK 061003 | | CD3 | |
| 83219 CC Well to Mod | 17.0 | 93769_Secondary Th2_anti-CD28/anti- | 24.0 |
| Diff (ODO3866) | | CD3 | |
| 83220 CC NAT | 8.9 | 93770_Secondary Tr1_anti-CD28/anti- | 18.7 |
| (ODO3866) | | CD3 | |
| 83221 CC Gr.2 | 7.8 | 93573_Secondary Th1_resting day 4-6 | 2.3 |
| rectosigmoid | | in IL-2 | |
| (ODO3868) | | | |
| 83222 CC NAT | 2.0 | 93572_Secondary Th2_resting day 4-6 | 4.3 |
| (ODO3868) | | in IL-2 | |
| 83235 CC Mod Duff | 9.8 | 93571_Secondary Tr1_resting day 4-6 | 2.8 |
| (ODO3920) | | in IL-2 | |
| 83236 CC NAT | 15.2 | 93568_primary Th1_anti-CD28/anti- | 21.3 |
| (ODO3920) | | CD3 | |
| 83237 CC Gr.2 ascend | 29.1 | 93569_primary Th2_anti-CD28/anti- | 20.6 |
| colon(ODO3921) | | CD3 | |
| 83238 CC NAT | 6.5 | 93570_primary Tr1_anti-CD28/anti- | 31.4 |
| (ODO3921) | | CD3 | |
| 83241 CC from Partial | 51.8 | 93565_primary Th1_resting dy 4-6 in | 16.4 |
| Hepatectomy | | IL-2 | |
| (ODO4309) | | | |
| 83242 Liver NAT | 65.8 | 93566_primary Th2_resting dy 4-6 in | 10.3 |
| (ODO4309) | | IL-2 | |
| 87472 Colon mets to | 8.4 | 93567_primary Tr1_resting dy 4-6 in | 14.2 |
| lung (OD04451-01) | | IL-2 | |
| 87473 Lung NAT | 13.5 | 93351_CD45RA CD4 | 13.3 |
| (OD04451-02) | | lymphocyte_anti-CD28/anti-CD3 | |
| Normal Prostate | 26.9 | 93352_CD45RO CD4_ | 11.7 |
| Clontech A+6546-1 | | lymphocyte anti-CD28/anti-CD3 | |
| 84140 Prostate Cancer | 18.7 | 93251_CD8 Lymphocytes_anti- | 6.8 |
| (OD04410) | | CD28/anti-CD3 | |
| 84141 Prostate NAT | 15.1 | 93353_chronic CD8 Lymphocytes | 5.9 |
| (OD04410) | | 2ry_resting dy 4-6 in IL-2 | |
| 87073 Prostate Cancer | 17.3 | 93574_chronic CD8 Lymphocytes | 10.5 |
| (OD04720-01) | | 2ry_activated CD3/CD28 | |
| 87074 Prostate NAT | 18.4 | 93354_CD4_none | 1.4 |
| (OD04720-02) | | | |
| Normal Lung GENPAK | 35.0 | 93252_Secondary Th1/Th2/Tr1_anti- | 11.5 |
| 061010 | | CD95 CH11 | |
| 83239 Lung Met to | 39.1 | 93103_LAK cells_resting | 66.0 |
| Muscle (ODO4286) | | | |
| 83240 Muscle NAT | 8.1 | 93788_LAK cells_IL-2 | 13.8 |
| (ODO4286) | | | |
| 84136 Lung Malignant | 28.6 | 93787_LAK cells_IL-2+IL-12 | 24.0 |
| Cancer (OD03126) | | | |
| 84137 Lung NAT | 19.5 | 93789_LAK cells_IL-2+IFN gamma | 24.5 |
| (OD03126) | | | |
| 84871 Lung Cancer | 20.0 | 93790_LAK cells_IL-2+IL-18 | 20.7 |
| (OD04404) | | | |
| 84872 Lung NAT | 11.9 | 93104_LAK cells_PMA/ionomycin | 14.1 |
| (OD04404) | | and IL-18 | |
| 84875 Lung Cancer | 13.8 | 93578_NK Cells IL-2_resting | 5.9 |
| (OD04565) | | | |
| 84876 Lung NAT | 3.9 | 93109_Mixed Lymphocyte | 35.6 |
| (OD04565) | | Reaction_Two Way MLR | |
| 85950 Lung Cancer | 17.8 | 93110_Mixed Lymphocyte | 23.2 |
| (OD04237-01) | | Reaction_Two Way MLR | |
| 85970 LungNAT | 33.0 | 93111_Mixed Lymphocyte | 7.7 |
| (OD04237-02) | | Reaction_Two Way MLR | |
| 83255 Ocular Mel Met | 5.8 | 93112_Mononuclear Cells | 7.4 |
| to Liver (ODO4310) | | (PBMCs)_resting | |
| 83256 Liver NAT | 100.0 | 93113_Mononuclear Cells | 100.0 |
| (ODO4310) | | (PBMCs)_PWM | |
| 84139 Melanoma Mets | 7.3 | 93114_Mononuclear Cells | 31.6 |
| to Lung (OD04321) | | (PBMCs)_PHA-L | |
| 84138 Lung NAT | 13.8 | 93249_Ramos (B cell)_none | 29.5 |
| (OD04321) | | | |
| Normal Kidney | 37.6 | 93250_Ramos (B cell)_ionomycin | 47.0 |
| GENPAK 061008 | | | |
| 83786 Kidney Ca, | 46.1 | 93349_B lymphocytes_PWM | 36.6 |
| Nuclear grade 2 | | | |
| (OD04338) | | | |
| 83787 Kidney NAT | 18.8 | 93350_B lymphocytes_CD40L and IL- | 8.5 |
| (OD04338) | | 4 | |
| 83788 Kidney Ca | 17.3 | 92665_EOL-1 (Eosinophil)_dbcAMP | 4.7 |
| Nuclear grade ½ | | differentiated | |
| (OD04339) | | | |
| 83789 Kidney NAT | 71.6 | 93248_EOL-1 | 15.8 |
| (OD04339) | | (Eosinophil)_dbcAMP/PMAionomycin | |
| 83790 Kidney Ca, Clear | 69.5 | 93356_Dendritic Cells_none | 30.1 |
| cell type (OD04340) | | | |
| 83791 Kidney NAT | 23.4 | 93355_Dendritic Cells_LPS 100 ng/ml | 34.4 |
| (OD04340) | | | |
| 83792 Kidney Ca, | 9.8 | 93775_Dendritic Cells_anti-CD40 | 39.5 |
| Nuclear grade 3 | | | |
| (OD04348) | | | |
| 83793 Kidney NAT | 32.8 | 93774_Monocytes_resting | 26.1 |
| (OD04348) | | | |
| 87474 Kidney Cancer | 10.6 | 93776_Monocytes_LPS 50 ng/ml | 88.9 |
| (OD04622-01) | | | |
| 87475 Kidney NAT | 5.4 | 93581_Macrophages_resting | 96.6 |
| (OD04622-03) | | | |
| 85973 Kidney Cancer | 27.9 | 93582_Macrophages_LPS 100 ng/ml | 68.8 |
| (OD04450-01) | | | |
| 85974 Kidney NAT | 20.5 | 93098_HUVEC (Endothelial)_none | 15.9 |
| (OD04450-03) | | | |
| Kidney Cancer | 6.2 | 93099_HUVEC (Endothelial)_starved | 26.6 |
| Kidney NAT Clontech | 18.3 | 93100_HUVEC (Endothelial)_IL-1b | 9.7 |
| 8120608 | | | |
| Kidney Cancer | 3.9 | 93779_HUVEC (Endothelial)_IFN | 15.5 |
| Clontech 8120613 | | gamma | |
| Kidney NAT Clontech | 10.6 | 93102_HUVEC (Endothelial)_TNF | 1.9 |
| 8120614 | | alpha+IFN gamma | |
| Kidney Cancer | 13.9 | 93101_HUVEC (Endothelial)_TNF | 7.0 |
| Clontech 9010320 | | alpha+IL4 | |
| Kidney NAT Clontech | 20.7 | 93781_HUVEC (Endothelial)_IL-11 | 5.8 |
| 9010321 | | | |
| Normal Uterus | 8.3 | 93583_Lung Microvascular | 16.6 |
| GENPAK 061018 | | Endothelial Cells_none | |
| Uterus Cancer | 33.4 | 93584_Lung Microvascular | 7.1 |
| GENPAK 064011 | | Endothelial Cells_TNFa (4 ng/ml) and | |
| | | IL1b (1 ng/ml) | |
| Normal Thyroid | 9.1 | 92662_Microvascular Derma1 | 58.6 |
| Clontech A+6570-1 | | endothelium_none | |
| Thyroid Cancer | 52.9 | 92663_Microsvasular Dermal | 16.8 |
| GENPAK 064010 | | endothelium_TNFa (4 ng/ml) and IL1b | |
| | | (1 ng/ml) | |
| Thyroid Cancer | 20.8 | 93773_Bronchial epithelium_TNFa (4 | 40.3 |
| INVITROGEN | | ng/ml) and IL1b (1 ng/ml) ** | |
| A302152 | | | |
| Thyroid NAT | 33.1 | 93347_Small Airway Epithelium_none | 14.6 |
| INVITROGEN | | | |
| A302153 | | | |
| Normal Breast | 13.1 | 93348_Small Airway | 92.0 |
| GENPAK 061019 | | Epithelium_TNFa (4 ng/ml) and IL1b | |
| | | (1 ng/ml) | |
| 84877 Breast Cancer | 19.5 | 92668_Coronary Artery SMC_resting | 22.1 |
| (OD04566) | | | |
| 85975 Breast Cancer | 29.9 | 92669_Coronary Artery SMC_TNFa (4 | 7.5 |
| (OD04590-01) | | ng/ml) and IL1b (1 ng/ml) | |
| 85976 Breast Cancer | 27.1 | 93107_astrocytes_resting | 8.0 |
| Mets (OD04590-03) | | | |
| 87070 Breast Cancer | 30.9 | 93108_astrocytes_TNFa (4 ng/ml) and | 16.7 |
| Metastasis (OD04655- | | IL1b (1 ng/ml) | |
| 05) | | | |
| GENPAK Breast | 16.2 | 92666_KU-812 (Basophil)_resting | 18.3 |
| Cancer 064006 | | | |
| Breast Cancer Res. | 15.0 | 92667_KU-812 | 69.3 |
| Gen. 1024 | | (Basophil)_PMA/ionoycin | |
| Breast Cancer Clontech | 48.4 | 93579_CCD1106 | 13.7 |
| 9100266 | | (Keratinocytes)_none | |
| Breast NAT Clontech | 15.5 | 93580_CCD1106 | 56.6 |
| 9100265 | | (Keratinocytes)_TNFa and IFNg** | |
| Breast Cancer | 36.4 | 93791_Liver Cirrhosis | 7.6 |
| INVITROGEN | | | |
| A209073 | | | |
| Breast NAT | 11.6 | 93792_Lupus Kidney | 4.5 |
| INVITROGEN | | | |
| A2090734 | | | |
| Normal Liver | 39.0 | 93577_NCI-H292 | 37.9 |
| GENPAK 061009 | | | |
| Liver Cancer GENPAK | 14.6 | 93358_NCI-H292_IL-4 | 51.8 |
| 064003 | | | |
| Liver Cancer Research | 18.9 | 93360_NCI-H292_IL-9 | 48.0 |
| Genetics RNA 1025 | | | |
| Liver Cancer Research | 11.0 | 93359_NCI-H292_IL-13 | 26.2 |
| Genetics RNA 1026 | | | |
| Paired Liver Cancer | 35.1 | 93357_NCI-H292_IFN gamma | 27.9 |
| Tissue Research | | | |
| Genetics RNA 6004-T | | | |
| Paired Liver Tissue | 13.2 | 93777_HPAEC_- | 14.0 |
| Research Genetics | | | |
| RNA 6004-N | | | |
| Paired Liver Cancer | 12.7 | 93778_HPAEC_IL-1 beta/TNA alpha | 7.3 |
| Tissue Research | | | |
| Genetics RNA 6005-T | | | |
| Paired Liver Tissue | 8.0 | 93254_Normal Human Lung | 14.7 |
| Research Genetics | | Fibroblast_none | |
| RNA 6005-N | | | |
| Normal Bladder | 33.7 | 93253_Normal Human Lung | 9.9 |
| GENPAK 061001 | | Fibroblast_TNFa (4 ng/ml) and IL-1b | |
| | | (1 ng/ml) | |
| Bladder Cancer | 7.0 | 93257_Normal Human Lung | 36.1 |
| Research Genetics | | Fibroblast_IL-4 | |
| RNA 1023 | | | |
| Bladder Cancer | 11.6 | 93256_Normal Human Lung | 27.9 |
| INVITROGEN | Fibroblast_IL-9 | | |
| A302173 | | | |
| 87071 Bladder Cancer | 38.8 | 93255_Normal Human Lung | 65.1 |
| (OD04718-01) | | Fibroblast_IL-13 | |
| 87072 Bladder Normal | 17.4 | 93258_Normal Human Lung | 66.9 |
| Adjacent (OD047 18- | | Fibroblast_IFN gamma | |
| 03) | | | |
| Normal Ovary Res. | 4.6 | 93106_Dermal Fibroblasts | 38.2 |
| Gen. | | CCD1070_resting | |
| Ovarian Cancer | 33.7 | 93361_Dermal Fibroblasts | 50.3 |
| GENPAK 064008 | | CCD1070_TNF alpha 4 ng/ml | |
| 87492 Ovary Cancer | 28.4 | 93105_Dermal Fibroblasts | 27.2 |
| (OD04768-07) | | CCD1070_IL-1 beta 1 ng/ml | |
| 87493 Ovary NAT | 5.7 | 93772_dermal fibroblast_IFN gamma | 7.6 |
| (OD04768-08) | | | |
| Normal Stomach | 19.6 | 93771_dermal fibroblast_IL-4 | 16.0 |
| GENPAK 061017 | | | |
| Gastric Cancer | 4.5 | 93259_IBD Colitis 1** | 9.8 |
| Clontech 9060358 | | | |
| NAT Stomach Clontech | 18.6 | 93260_IBD Colitis 2 | 1.1 |
| 9060359 | | | |
| Gastric Cancer | 34.2 | 93261_IBD Crohns | 3.5 |
| Clontech 9060395 | | | |
| NAT Stomach Clontech | 13.9 | 735010_Colon_normal | 38.7 |
| 9060394 | | | |
| Gastric Cancer | 48.7 | 735019_Lung_none | 24.1 |
| Clontech 9060397 | | | |
| NAT Stomach Clontech | 3.4 | 64028-1_Thymus_none | 94.0 |
| 9060396 | | | |
| Gastric Cancer | 30.6 | 64030-1_Kidney_none | 10.2 |
| GENPAK 064005 | | | |

As is shown in Table 16N, (Panel 1.2), POLY12 is expressed in a variety of tissues. As is shown in Table 160 (Panel 2D), POLY12 is expressed in a variety of cancers. As is shown in Table 160 (Panel 4D), POLY12 is upregulated in several normal and activated tissues. POLY 12 is particularly high in activated monocytes and both activated and resting macrophages. Thus POLY12 may serve as a marker for differentiating monocytes and macrophages, resting and activated. Antagonistic therapeutics to this molecule may inhibit the differentiation process, activation of the epithelium or keratinocytes in the skin and block or lessen inflammation in diseases such as asthma, allergy, psoriasis and emphysema.

H. POLY13.

Quantitative expression of POLY13 was assessed as described in Example 4. was assessed using the primer-probe set Ag1161, described in Table 16P. Results of the RTQ-PCR runs are shown in Table 16Q.

TABLE 16P
Probe Name: Ag1161

| | | | | | Start |
|---|---|---|---|---|---|
| Primers | Sequences | | TM | Length | Position |
| Forward | 5′-AACTCCAAGGTCGCCTTCT-3′ | (SEQ ID NO.:69) | 58.9 | 19 | 205 |
| Probe | FAM-5′-AACCACGAGCCATCCGAGATG | (SEQ ID NO.:70) | 68.8 | 23 | 241 |
| | AG-3′-TAMRA | | | | |
| Reverse | 5′-AGTAAATGATGCGCGTCTTGT-3′ | (SEQ ID NO.:71) | 59.8 | 21 | 266 |

TABLE 16Q
Panels 1.2 and 4D

| PANEL 1.2 | | PANEL 4D | |
|---|---|---|---|
| | Relative | | Relative |
| | Expression (%) | | Expression (%) |
| | 1.2tm1385f_ag | | 4Dtm1977f_ag |
| Tissue Name | 1161 | Tissue Name | 1161 |
| Endothelial cells | 0.0 | 93768_Secondary Th1_anti-CD28/anti- | 0.0 |
| | | CD3 | |
| Endothelial cells | 0.2 | 93769_Secondary Th2_anti-CD28/anti- | 0.0 |
| (treated) | | CD3 | |
| Pancreas | 0.2 | 93770_Secondary Tr1_anti-CD28/anti- | 0.0 |
| | | CD3 | |
| Pancreatic ca. | 0.0 | 93573_Secondary Th1_resting day 4-6 | 0.0 |
| CAPAN 2 | | in IL-2 | |
| Adrenal Gland (new | 18.9 | 93572_Secondary Th2_resting day 4-6 | 0.0 |
| lot*) | | in IL-2 | |
| Thyroid | 0.0 | 93571_Secondary Tr1_resting day 4-6 | 0.0 |
| | | in IL-2 | |
| Salivary gland | 0.2 | 93568_primary Th1_anti-CD28/anti- | 0.0 |
| | | CD3 | |
| Pituitary gland | 0.0 | 93569_primary Th2_anti-CD28/anti- | 0.0 |
| | | CD3 | |
| Brain (fetal) | 1.2 | 93570_primary Tr1_anti-CD28/anti- | 0.0 |
| | | CD3 | |
| Brain (whole) | 24.1 | 93565_primary Th1_resting dy 4-6 in | 0.0 |
| | | IL-2 | |
| Brain (amygdala) | 36.1 | 93566_primary Th2_resting dy 4-6 in | 0.0 |
| | | IL-2 | |
| Brain (cerebellum) | 0.4 | 93567_primary Tr1_resting dy 4-6 in | 0.0 |
| | | IL-2 | |
| Brain (hippocampus) | 21.9 | 93351_CD45RA CD4 | 0.0 |
| | | lymphocyte anti-CD28/anti-CD3 | |
| Brain (thalamus) | 11.7 | 93352_CD45RO CD4 | 0.0 |
| | | lymphocyte_anti-CD28/anti-CD3 | |
| Cerebral Cortex | 100.0 | 93251_CD8 Lymphocytes_anti- | 0.0 |
| | | CD28/anti-CD3 | |
| Spinal cord | 0.4 | 93353_chronic CD8 Lymphocytes | 0.0 |
| | | 2ry_resting dy 4-6 in IL-2 | |
| CNS ca. (glio/astro) | 0.0 | 93574_chronic CD8 Lymphocytes | 0.0 |
| U87-MG | | 2ry_activated CD3/CD28 | |
| CNS ca. (glio/astro) | 0.0 | 93354_CD4_none | 0.0 |
| U-118-MG | | | |
| CNS ca. (astro) | 0.0 | 93252_Secondary Th1/Th2/Tr1_anti- | 0.0 |
| SW1783 | | CD95 CH11 | |
| CNS ca.* (neuro; met) | 0.0 | 93103_LAK cells_resting | 0.0 |
| SK-N-AS | | | |
| CNS ca. (astro) | 0.0 | 93788_LAK cells_IL-2 | 0.0 |
| SF-539 | | | |
| CNS ca. (astro) | 0.0 | 93787_LAK cells_IL-2+IL-12 | 0.0 |
| SNB-75 | | | |
| CNS ca. (glio) | 0.0 | 93789_LAK cells_IL-2+IFN gamma | 0.0 |
| SNB-19 | | | |
| CNS ca. (glio) | 0.0 | 93790_LAK cells_IL-2+IL-18 | 0.0 |
| U251 | | | |
| CNS ca. (glio) | 0.0 | 93104_LAK cells_PMA/ionomycin and | 0.0 |
| SF-295 | | IL-18 | |
| Heart | 0.3 | 93578_NK Cells IL-2_resting | 0.0 |
| Skeletal Muscle (new | 1.1 | 93109_Mixed Lymphocyte | 0.0 |
| lot*) | | Reaction_Two Way MLR | |
| Bone marrow | 0.0 | 93110_Mixed Lymphocyte | 0.0 |
| | | Reaction_Two Way MLR | |
| Thymus | 0.0 | 93111_Mixed Lymphocyte | 0.0 |
| | | Reaction_Two Way MLR | |
| Spleen | 0.0 | 93112_Mononuclear Cells | 0.0 |
| | | (PBMCs)_resting | |
| Lymph node | 0.0 | 93113_Mononuclear Cells | 0.0 |
| | | (PBMCs)_PWM | |
| Colorectal | 0.0 | 93114_Mononuclear Cells | 0.0 |
| | | (PBMCs)_PHA-L | |
| Stomach | 0.2 | 93249_Ramos (B cell)_none | 0.0 |
| Small intestine | 0.3 | 93250_Ramos (B cell)_ionomycin | 0.0 |
| Colon ca. | 0.0 | 93349_B lymphocytes_PWM | 0.0 |
| SW480 | | | |
| Colon ca.* (SW480 | 0.0 | 93350_B lymphocytes_CD40L and IL- | 0.0 |
| met)SW620 | | 4 | |
| Colon ca. | 0.0 | 92665_EOL-1 (Eosinophil)_dbcAMP | 0.0 |
| HT29 | | differentiated | |
| Colon ca. | 0.0 | 93248_EOL-1 | 0.0 |
| HCT-116 | | (Eosinophil)_dbcAMP/PMAionomycin | |
| Colon ca. | 0.0 | 93356_Dendritic Cells_none | 0.0 |
| CaCo-2 | | | |
| 83219 CC Well to Mod | 0.2 | 93355_Dendritic Cells_LPS 100 ng/ml | 0.0 |
| Diff (ODO3866) | | | |
| Colon ca. | 0.0 | 93775_Dendritic Cells_anti-CD40 | 0.0 |
| HCC-2998 | | | |
| Gastric ca.* (liver met) | 0.0 | 93774_Monocytes resting | 0.0 |
| NCI-N87 | | | |
| Bladder | 0.5 | 93776_Monocytes_LPS 50 ng/ml | 0.0 |
| Trachea | 0.2 | 93581_Macrophages_resting | 0.0 |
| Kidney | 0.4 | 93582_Macrophages_LPS 100 ng/ml | 18.2 |
| Kidney (fetal) | 1.0 | 93098_HUVEC (Endothelial)_none | 0.0 |
| Renal Ca. | 0.0 | 93099_HUVEC (Endothelial)_starved | 0.0 |
| 786-0 | | | |
| Renal ca | 0.0 | 93100_HUVEC (Endothelial)_IL-1b | 0.0 |
| A498 | | | |
| Renal ca. | 0.0 | 93779_HUVEC (Endothelial)_IFN | 0.0 |
| RXF 393 | | gamma | |
| Renal ca. | 0.0 | 93102_HUVEC (Endothelial)_TNF | 0.0 |
| ACHN | | alpha+IFN gamma | |
| Renal ca. | 0.0 | 93101_HUVEC (Endothelial)_TNF | 0.0 |
| UO-31 | | alpha+IL4 | |
| Renal ca. | 0.0 | 93781_HUVEC (Endothelial)_IL-11 | 0.0 |
| TK-10 | | | |
| Liver | 0.4 | 93583_Lung Microvascular Endothelial | 0.0 |
| | | Cells_none | |
| Liver (fetal) | 0.2 | 93584_Lung Microvascular Endothelial | 0.0 |
| | | Cells_TNFa (4 ng/ml) and IL1b (1 | |
| | | ng/ml) | |
| Liver ca. (hepatoblast) | 0.0 | 92662_Microvascular Dermal | 0.0 |
| HepG2 | | endothelium_none | |
| Lung | 0.2 | 92663_Microsvasular Dermal | 0.0 |
| | | endothelium_TNFa (4 ng/ml) and IL1b | |
| | | (1 ng/ml) | |
| Lung (fetal) | 0.0 | 93773_Bronchial epithelium_TNFa (4 | 0.0 |
| | | ng/ml) and IL1b (1 ng/ml) ** | |
| Lung Ca. (small cell) | 0.0 | 93347_Small Airway Epithelium_none | 0.0 |
| LX-1 | | | |
| Lung ca. (small cell) | 0.0 | 93348_Small Airway Epithelium_TNFa | 0.0 |
| NCI-H69 | | (4 ng/ml) and IL1b (1 ng/ml) | |
| Lung ca. (s.cell var.) | 0.0 | 92668_Coronery Artery SMC_resting | 0.0 |
| SHP-77 | | | |
| Lung ca. (large | 0.3 | 92669_Coronery Artery SMC_TNFa (4 | 0.0 |
| cell)NCI-H460 | | ng/ml) and IL1b (1 ng/ml) | |
| Lung ca. (non-sm. cell) | 0.0 | 93107_astrocytes_resting | 0.0 |
| A549 | | | |
| Lung ca. (non-s.cell) | 0.0 | 93108_astrocytes_TNFa (4 ng/ml) and | 0.0 |
| NCI-H23 | | IL1b (1 ng/ml) | |
| Lung ca (non-s.cell) | 0.0 | 92666_KU-812 (Basophil)_resting | 0.0 |
| HOP-62 | | | |
| Lung ca. (non-s.cl) | 0.0 | 92667_KU-812 | 0.0 |
| NCI-H522 | | (Basophil)_PMA/ionoycin | |
| Lung ca. (squam.) SW | 0.0 | 93579_CCD1106 (Keratinocytes)₁₃none | 0.0 |
| 900 | | | |
| Lung ca. (squam.) | 0.0 | 93580_CCD1106 | 0.0 |
| NCI-H596 | | (Keratinocytes)_TNFa and IFNg** | |
| Mammary gland | 0.5 | 93791_Liver Cirrhosis | 68.3 |
| Breast ca.* (pl. | 0.0 | 93792_Lupus Kidney | 26.6 |
| effusion) MCF-7 | | | |
| Breast ca.* (pl.ef) | 0.0 | 93577_NCI-H292 | 0.0 |
| MDA-MB-231 | | | |
| Breast ca.* (pl. | 0.0 | 93358_NCI-H292_IL-4 | 0.0 |
| effusion) T47D | | | |
| Breast ca. | 0.0 | 93360_NCI-H292_IL-9 | 0.0 |
| BT-549 | | | |
| Breast ca. | 0.0 | 93359_NCI-H292_IL-13 | 0.0 |
| MDA-N | | | |
| Ovary | 1.8 | 93357_NCI-H292_IFN gamma | 0.0 |
| Ovarian ca. | 0.0 | 93777_HPAEC_ | 0.0 |
| OVCAR-3 | | | |
| Ovarian ca. | 0.0 | 93778_HPAEC_IL-1 beta/TNA alpha | 0.0 |
| OVCAR-4 | | | |
| Ovarian ca. | 0.0 | 93254_Normal Human Lung | 0.0 |
| OVCAR-5 | | Fibroblast_none | |
| Ovarian ca. | 0.0 | 93253_Normal Human Lung | 0.0 |
| OVCAR-8 | | Fibroblast_TNFa (4 ng/ml) and IL-1b | |
| | | (1 ng/ml) | |
| Ovarian ca. | 0.0 | 93257_Normal Human Lung | 0.0 |
| IGROV-1 | | Fibroblast_IL-4 | |
| Ovarian ca.* (ascites) | 0.0 | 93256_Normal Human Lung | 0.0 |
| SK-OV-3 | | Fibroblast_IL-9 | |
| Uterus | 0.3 | 93255_Normal Human Lung | 0.0 |
| | | Fibroblast_IL-13 | |
| Placenta | 0.0 | 93258_Normal Human Lung | 0.0 |
| | | Fibroblast_IFN gamma | |
| Prostate | 0.2 | 93106_Dermal Fibroblasts | 0.0 |
| | | CCD1070_resting | |
| Prostate ca.* (bone | 0.0 | 93361_Dermal Fibroblasts | 0.0 |
| met)PC-3 | | CCD1070_TNF alpha 4 ng/ml | |
| Testis | 1.7 | 93105_Dermal Fibroblasts | 0.0 |
| | | CCD1070_IL-1 beta 1 ng/ml | |
| Melanoma | 0.0 | 93772_dermal fibroblast_IFN gamma | 0.0 |
| Hs688(A).T | | | |
| Melanoma* (met) | 0.0 | 93771_dermal fibroblast_IL-4 | 0.0 |
| Hs688(B).T | | | |
| Melanoma | 0.0 | 93259_IBD Colitis 1** | 93.3 |
| UACC-62 | | | |
| Melanoma | 0.0 | 93260_IBD Colitis 2 | 0.0 |
| M14 | | | |
| Melanoma | 0.0 | 93261_IBD Crohns | 8.8 |
| LOX IMVI | | | |
| Melanoma* (met) SK- | 0.0 | 735010_Colon_normal | 81.8 |
| MEL-5 | | | |
| Adipose | 3.8 | 735019_Lung_none | 100.0 |
| | | 64028-1_Thymus_none | 26.2 |
| | | 64030-1_Kidney_none | 25.9 |

As is shown in Table 16Q (Panel 1.2), POLY13 expression is highest in the brain, with a significant low level expression in adrenal glands. Decreased expressions are seen in adipose with much lower levels in the testis, ovary, skeletal muscle and fetal kidney. In the brain, a clear distinction is seen between adult and fetal brain, with expression levels being more than 20-fold higher in adult brain relative to fetal brain. There are also differences of expression between the various regions of the brain. Cerebrum shows highest levels of expression, followed by amygdala, hippocampus and thalamus. Interestingly, cerebellum shows extremely low levels of expression as compared to other regions of the brain. This is in contrast to a POLY13 homolog, cerebellin, which is a marker of cerebellar Purkinje cells. In addition, all of the CNS cancer cell lines represented on this panel have low to undetectable expression of this gene. Therefore this gene may be used as a marker potentially for normal brain, or to distinguish, for example, cerebrum versus cerebellum. Cerebellin appears to act as a neuromodulator and has homology to members of the complement cascade. POLY 13 may therefore play a role in selective transmission in certain brain regions or be involved in immune modulation in the CNS.

As is shown in Table 16Q (Panel 4D) POLY13 is expressed in the lung, colon and in the liver. The level of expresssion in colon during Crohn's disease is reduced suggesting that protein therapeutics derived from the protein encoded for by this transcript may reduce or eliminate inflammation in Crohn's disease or inflammatory bowel disease. The high expression in IBD colitis may be from genomic DNA contamination.

I. POLY14.

Quantitative expression of POLY14 was assessed using the primer-probe set Ag1206, described in Table 16R. Results of the RTQ-PCR runs are shown in Table 16S.

TABLE 16R
Probe Name: Ag1206

| | | | | | Start |
|---|---|---|---|---|---|
| Primers | Sequences | | TM | Length | Position |
| Forward | 5′-TGAATGACTTCGAGGTGCTC-3′ | (SEQ ID NO.:72) | 59 | 20 | 95 |
| Probe | FAM-5′-CACAGAGCTACAGCGGCTGCT | (SEQ ID NO.:73) | 69.3 | 26 | 120 |
| | ACAAG-3′-TAMRA | | | | |
| Reverse | 5′-CTCTTCAGCATCTGCCACAT-3′ | (SEQ ID NO.:74) | 59 | 20 | 192 |

TABLE 16S
Panels 1.2 and 4D

| | Relative | | Relative |
|---|---|---|---|
| | Expression (%) | | Expression (%) |
| | 1.2tm1400f_ag | | 4Dtm2063f_ag |
| Tissue Name | 1206 | Tissue Name | 1206 |
| Endothelial cells | 0.0 | 93768_Secondary Th1_1anti-CD28/anti-CD3 | 1.7 |
| Endothelial cells | 0.2 | 93769_Secondary Th2_anti-CD28/anti-CD3 | 2.3 |
| (treated) | | | |
| Pancreas | 9.0 | 93770_Secondary Tr1_anti-CD28/anti-CD3 | 4.8 |
| Pancreatic ca. | 0.0 | 93573_Secondary Th1_resting day 4-6 in IL- | 0.7 |
| CAPAN 2 | | 2 | |
| Adrenal Gland | 4.0 | 93572_Secondary Th2_resting day 4-6 in IL- | 1.2 |
| (new lot*) | | 2 | |
| Thyroid | 0.0 | 93571_Secondary Tr1_resting day 4-6 in IL- | 0.4 |
| | | 2 | |
| Salivary gland | 0.4 | 93568_primary Th1_anti-CD28/anti-CD3 | 1.1 |
| Pituitary gland | 0.7 | 93569_primary Th2_anti-CD28/anti-CD3 | 1.0 |
| Brain (fetal) | 0.2 | 93570_primary Tr1_anti-CD28/anti-CD3 | 1.3 |
| Brain (whole) | 0.3 | 93565_primary Th1_resting dy 4-6 in IL-2 | 8.8 |
| Brain (amygdala) | 0.0 | 93566_primary Th2_resting dy 4-6 in IL-2 | 8.6 |
| Brain (cerebellum) | 0.0 | 93567_primary Tr1_resting dy 4-6 in IL-2 | 4.5 |
| Brain | 0.0 | 93351_CD45RA CD4 lymphocyte_anti- | 1.0 |
| (hippocampus) | | CD28/anti-CD3 | |
| Brain (thalamus) | 0.0 | 93352_CD45RO CD4 lymphocyte_anti- | 6.8 |
| | | CD28/anti-CD3 | |
| Cerebral Cortex | 0.0 | 93251_CD8 Lymphocytes_anti-CD28/anti- | 2.0 |
| | | CD3 | |
| Spinal cord | 0.5 | 93353_chronic CD8 Lymphocytes | 0.7 |
| | | 2ry_resting dy 4-6 in IL-2 | |
| CNS ca. | 0.0 | 93574_chronic CD8 Lymphocytes | 2.2 |
| (glio/astro) | | 2ry_activated CD3/CD28 | |
| U87-MG | | | |
| CNS ca. | 0.0 | 93354_CD4_none | 4.7 |
| (glio/astro) U- | | | |
| 118-MG | | | |
| CNS ca. (astro) | 0.0 | 93252_Secondary Th1/Th2/Tr1_anti-CD95 | 5.6 |
| SW1783 | | CH11 | |
| CNS ca.* (neuro; | 0.0 | 93103_LAK cells_resting | 2.5 |
| met) SK-N-AS | | | |
| CNS ca. (astro) | 0.0 | 93788_LAK cells_IL-2 | 2.9 |
| SF-539 | | | |
| CNS ca. (astro) | 0.0 | 93787_LAK cells_IL-2+IL-12 | 1.3 |
| SNB-75 | | | |
| CNS ca. (glio) | 0.3 | 93789_LAK cells_IL-2+IFN gamma | 3.0 |
| SNB-19 | | | |
| CNS ca. (glio) | 0.0 | 93790_LAK cells_IL-2+IL-18 | 1.4 |
| U251 | | | |
| CNS Ca. (glio) | 0.0 | 93104_LAK cells_PMA/ionomycin and IL- | 2.8 |
| SF-295 | | 18 | |
| Heart | 0.0 | 93578_NK Cells IL-2_resting | 2.1 |
| Skeletal Muscle | 0.0 | 93109_Mixed Lymphocyte Reaction_Two | 5.1 |
| (new lot*) | | Way MLR | |
| Bone marrow | 0.0 | 93110_Mixed Lymphocyte Reaction_Two | 1.2 |
| | | Way MLR | |
| Thymus | 0.0 | 93111_Mixed Lymphocyte Reaction_Two | 1.5 |
| | | Way MLR | |
| Spleen | 0.0 | 93112_Mononuclear Cells (PBMCs)_resting | 1.4 |
| Lymph node | 0.0 | 93113_Mononuclear Cells (PBMCs)_PWM | 3.2 |
| Colorectal | 0.0 | 93114_Mononuclear Cells (PBMCs)_PHA-L | 1.3 |
| Stomach | 1.3 | 93249_Ramos (B cell)_none | 2.1 |
| Small intestine | 6.4 | 93250_Ramos (B cell)_ionomycin | 2.5 |
| Colon ca. | 0.0 | 93349_B lymphocytes_PWM | 0.6 |
| SW480 | | | |
| Colon ca.* | 0.0 | 93350_B lymphocytes_CD40L and IL-4 | 1.9 |
| (SW480 | | | |
| met)SW620 | | | |
| Colon ca. | 0.0 | 92665_EOL-1 (Eosinophil)_dbcAMP | 7.4 |
| HT29 | | differentiated | |
| Colon ca. | 0.7 | 93248_EOL-1 | 2.4 |
| HCT-116 | | (Eosinophil)_dbcAMP/PMAionomycin | |
| Colon ca. | 2.7 | 93356_Dendritic Cells_none | 3.6 |
| CaCo-2 | | | |
| 83219 CC Well to | 0.0 | 93355_Dendritic Cells_LPS 100 ng/ml | 1.9 |
| Mod Diff | | | |
| (ODO3866) | | | |
| Colon ca. | 1.8 | 93775_Dendritic Cells_anti-CD40 | 1.9 |
| HCC-2998 | | | |
| Gastric ca.* (liver | 2.0 | 93774_Monocytes_resting | 2.4 |
| met) NCI-N87 | | | |
| Bladder | 1.2 | 93776_Monocytes_LPS 50 ng/ml | 0.7 |
| Trachea | 0.0 | 93581_Macrophages_resting | 3.0 |
| Kidney | 1.7 | 93582_Macrophages_LPS 100 ng/ml | 2.0 |
| Kidney (fetal) | 1.5 | 93098_HUVEC (Endothelial)_none | 0.0 |
| Renal Ca. | 0.0 | 93099_HUVEC (Endothelial)_starved | 0.8 |
| 786-0 | | | |
| Renal ca. | 0.2 | 93100_HUVEC (Endothelial)_IL-1b | 0.0 |
| A498 | | | |
| Renal Ca. | 0.0 | 93779_HUVEC (Endothelial)_IFN gamma | 1.9 |
| RXF 393 | | | |
| Renal Ca. | 0.0 | 93102_HUVEC (Endothelial)_TNF alpha+ | 0.6 |
| ACHN | | IFN gamma | |
| Renal ca. | 0.0 | 93101_HUVEC (Endothelial)_TNF alpha+ | 0.0 |
| UO-31 | | IL4 | |
| Renal ca. | 0.0 | 93781_HUVEC (Endothelial)_IL-11 | 0.6 |
| TK-10 | | | |
| Liver | 84.1 | 93583_Lung Microvascular Endothelial | 0.4 |
| | | Cells_none | |
| Liver (fetal) | 32.1 | 93584_Lung Microvascular Endothelial | 1.5 |
| | | Cells_TNFa (4 ng/ml) and IL1b (1 ng/ml) | |
| Liver Ca. | 100.0 | 92662_Microvascular Dermal | 4.0 |
| (hepatoblast) | | endothelium_none | |
| HepG2 | | | |
| Lung | 0.0 | 92663_Microsvasular Dermal | 0.9 |
| | | endothelium_TNFa (4 ng/ml) and IL1b (1 | |
| | | ng/ml) | |
| Lung (fetal) | 0.2 | 93773_Bronchial epithelium_TNFa (4 ng/ml) | 9.3 |
| | | and IL1b(1 ng/ml) ** | |
| Lung ca. (small | 0.4 | 93347_Small Airway Epithelium_none | 1.1 |
| cell) LX-1 | | | |
| Lung ca. (small | 0.0 | 93348_Small Airway Epithelium_TNFa (4 | 4.4 |
| cell) NCI-H69 | | ng/ml) and IL1b (1 ng/ml) | |
| Lung ca. (s.cell | 0.0 | 92668_Coronery Artery SMC_resting | 0.8 |
| var.) SHP-77 | | | |
| Lung ca. (large | 0.8 | 92669_Coronery Artery SMC_TNFa (4 | 1.4 |
| cell)NCI-H460 | | ng/ml) and IL1b (1 ng/ml) | |
| Lung ca. (non-sm. | 0.0 | 93107_astrocytes_resting | 2.3 |
| cell) A549 | | | |
| Lung ca. (non- | 0.1 | 93108_astrocytes_TNFa (4 ng/ml) and IL1b | 3.3 |
| s.cell) NCI-H23 | | (1 ng/ml) | |
| Lung ca (non- | 0.0 | 92666_KU-812 (Basophil)_resting | 0.6 |
| s.cell) HOP-62 | | | |
| Lung ca. (non-s.cl) | 4.4 | 92667_KU-812 (Basophil)_PMA/ionoycin | 0.3 |
| NCI-H522 | | | |
| Lung ca. (squam.) | 0.0 | 93579_CCD1106 (Keratinocytes)_none | 0.5 |
| SW900 | | | |
| Lung ca. (squam.) | 0.0 | 93580_CCD1106 (Keratinocytes)_TNFa and | 0.5 |
| NCI-H596 | | IFNg** | |
| Mammary gland | 0.7 | 93791_Liver Cirrhosis | 100.0 |
| Breast ca.* (pl. | 0.0 | 93792_Lupus Kidney | 1.0 |
| effusion) MCF-7 | | | |
| Breast ca.* (pl.ef) | 0.0 | 93577_NCI-H292 | 9.4 |
| MDA-MB-231 | | | |
| Breast ca.* (pl. | 2.3 | 93358_NCI-H292_IL-4 | 3.7 |
| effusion) T47D | | | |
| Breast ca. | 0.0 | 93360_NCI-H292_IL-9 | 9.7 |
| BT-549 | | | |
| Breast ca. | 0.0 | 93359_NCI-H292_IL-13 | 5.2 |
| MDA-N | | | |
| Ovary | 0.0 | 93357_NCI-H292_IFN gamma | 4.0 |
| Ovarian ca. | 0.0 | 93777_HPAEC_- | 4.2 |
| OVCAR-3 | | | |
| Ovarian ca. | 0.0 | 93778_HPAEC_IL-1 beta/TNA alpha | 0.6 |
| OVCAR-4 | | | |
| Ovarian ca. | 0.2 | 93254_Normal Human Lung Fibroblast_none | 2.6 |
| OVCAR-5 | | | |
| Ovarian ca. | 0.0 | 93253_Normal Human Lung | 6.9 |
| OVCAR-8 | | Fibroblast_TNFa (4 ng/ml) and IL-1b (1 | |
| | | ng/ml) | |
| Ovarian ca. | 1.7 | 93257_Normal Human Lung Fibroblast_IL-4 | 0.8 |
| IGROV-1 | | | |
| Ovarian ca.* | 0.0 | 93256_Normal Human Lung Fibroblast_IL-9 | 0.4 |
| (ascites) SK-OV-3 | | | |
| Uterus | 0.0 | 93255_Normal Human Lung Fibroblast_IL- | 1.1 |
| | | 13 | |
| Placenta | 0.8 | 93258_Normal Human Lung Fibroblast_IFN | 4.6 |
| | | gamma | |
| Prostate | 0.0 | 93106_Dermal Fibroblasts CCD1070_resting | 3.1 |
| Prostate ca.* (bone | 1.4 | 93361_Dermal Fibroblasts CCD1070_TNF | 6.6 |
| met)PC-3 | | alpha 4 ng/ml | |
| Testis | 17.2 | 93105_Dermal Fibroblasts CCD1070_IL-1 | 2.3 |
| | | beta 1 ng/ml | |
| Melanoma | 0.0 | 93772_dermal fibroblast_IFN gamma | 0.5 |
| Hs688(A).T | | | |
| Melanoma* (met) | 0.0 | 93771_dermal fibroblast_IL-4 | 2.7 |
| Hs688(B).T | | | |
| Melanoma | 0.0 | 93259_IBD Colitis 1** | 5.9 |
| UACC-62 | | | |
| Melanoma | 0.0 | 93260_IBD Colitis 2 | 0.9 |
| M14 | | | |
| Melanoma | 0.0 | 93261_IBD Crohns | 0.7 |
| LOX IMVI | | | |
| Melanoma* (met) | 0.0 | 735010_Colon_normal | 12.7 |
| SK-MEL-5 | | | |
| Adipose | 0.4 | 735019_Lung_none | 1.7 |
| | | 64028-1_Thymus_none | 64.2 |
| | | 64030-1_Kidney_none | 5.6 |

As is shown in Table 16S (Panel 1.2), POLY14 is expressed at very high levels in liver with higher expression in adult than in fetal liver. Lower levels of expression are seen in testis, pancreas, small intestine, adrenal gland, kidney, stomach, bladder and pituitary, and expression in the brain, salivary gland, spinal cord, and adipose being still lower. Furtherrnore, this gene is expressed at modest levels in certain kinds of prostate cancer, ovarian cancer, breast cancer and lung cancer, when it is expressed at low or undetectable levels in the corresponding normal tissues.

As is shown in Table 16S (Panel 4D), POLY 14 is highly expressed in liver with cirrhosis and in normal thymus. This transcript may encode a protein that is in the stromal component of these tissues. Therapeutics designed to regulate the expression or function of POLY14 may detect, reduce or prevent liver cirrhosis and may be able to regulate T cell production in the thymus.

Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
(a) a mature form of an amino acid sequence selected from the group consisting of SEQ ID NO:2, 4, 6, 8, 10, 12, 14, 16, 18, 20, 22, 24, 26 and/or 28;
(b) a variant of a mature form of an amino acid sequence selected from the group consisting of SEQ ID NO:2, 4, 6, 8, 10, 12, 14, 16, 18, 20, 22, 24, 26 and/or 28, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 12, 14, 16, 18, 20, 22, 24, 26 and/or 28; and
(d) a variant of an amino acid sequence selected from the group consisting of SEQ ID NO:2, 4, 6, 8, 10, 12, 14, 16, 18, 20, 22, 24, 26 and/or 28 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NO:2, 4, 6, 8, 10, 12, 14, 16, 18, 20, 22, 24, 26 and/or 28.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:1, 3,5,7,9, 11, 13, 15, 17, 19,21,23,25 and/or 27.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO:2, 4, 6, 8, 10, 12, 14, 16, 18,20, 22, 24, 26 and/or 28;
(b) a variant of a mature form of an amino acid sequence selected from the group consisting of SEQ ID NO: 2, 4, 6, 8, 10, 12, 14, 16, 18, 20, 22, 24, 26 and/or 28 wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO: 2, 4, 6, 8, 10, 12, 14, 16, 18, 20, 22, 24, 26 and/or 28;
(d) a variant of an amino acid sequence selected from the group consisting of SEQ ID NO: 2, 4, 6, 8, 10, 12, 14, 16, 18, 20, 22, 24, 26 and/or 28, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO: 2, 4, 6, 8, 10, 12, 14, 16, 18, 20, 22, 24, 26 and/or 28,, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:1, 3, 5, 7, 9, 11, 13, 15, 17, 19, 21, 23, 25 and/or 27.

9. The nucleic acid molecule of claim 5, wherein said nucleic acid molecule comprises a nucleotide sequence selected from the group consisting of
(a) a nucleotide sequence selected from the group consisting of SEQ ID NO:1, 3,5,7,9, 11, 13, 15,17, 19,21,23,25 and/or 27;
(b) a nucleotide sequence differing by one or more nucleotides from a nucleotide sequence selected from the group consisting of SEQ ID NO: 1,3, 5, 7, 9, 11, 13, 15, 17, 19, 21, 23, 25 and/or 27, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NO: 1, 3, 5, 7, 9, 11, 13, 15, 17, 19, 21, 23, 25 and/or 27, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a POLYX-associated disorder, said method comprising administering to a subject in which such treatment or prevention is desired the polypeptide of claim 1 in an amount sufficient to treat or prevent said POLYX-associated disorder in said subject.

24. The method of claim 23, wherein said subject is a human.

25. A method of treating or preventing a POLYX-associated disorder, said method comprising administering to a subject in which such treatment or prevention is desired the nucleic acid of claim 5 in an amount sufficient to treat or prevent said POLYX-associated disorder in said subject.

26. The method of claim 25, wherein said subject is a human.

27. A method of treating or preventing a POLYX-associated disorder, said method comprising administering to a subject in which such treatment or prevention is desired the antibody of claim 15 in an amount sufficient to treat or prevent said POLYX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a POLYX-associated disorder, wherein said therapeutic is selected from the group consisting of a POLYX polypeptide, a POLYX nucleic acid, and a POLYX antibody.

36. A method for screening for a modulator of activity or of latency or predisposition to a POLYX-associated disorder, said method comprising:
(a) administering a test compound to a test animal at increased risk for a POLYX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a POLYX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO:2, 4, 6, 8, 10, 12, 14, 16, 18, 20, 22, 24, 26 and/or 28, or a biologically active fragment thereof.

41. A method of treating a pathological state in a mammal, the method comprising administering to the mammal the antibody of claim 15 in an amount sufficient to alleviate the pathological state.
